(19) United States
(12) Patent Application Publication
(10) Pub. No.: US 2012/0058527 A1
(43) Pub. Date: Mar. 8, 2012

(54) CAL A-RELATED ACYLTRANSFERASES AND METHODS OF USE, THEREOF

(75) Inventor: Susan Madrid, South San Francisco, CA (US)

(73) Assignee: Danisco US Inc., Palo Alto, CA (US)

(21) Appl. No.: 13/257,826

(22) PCT Filed: Mar. 19, 2010
(86) PCT No.: PCT/US2010/028001

§ 371 Date: Nov. 4, 2011

Related U.S. Application Data
(60) Provisional application No. 61/162,455, filed on Mar. 23, 2009.

(51) Int. Cl. C12N 9/10; C12N 15/63; C12P 7/62; C12P 7/64; C12S 11/00; C12P 7/40

(52) U.S. Cl. 435/134; 435/193; 435/264; 435/263; 435/136; 435/135; 435/320.1

(57) ABSTRACT

Compositions and methods relating to lipase/acyltransferase enzymes identified in prokaryotes and eukaryotes are described. These enzymes can be used in such applications as lipid stain removal from fabrics and hard surfaces and chemical synthesis reactions.

### PRIORITY

The present application claims priority to U.S. Provisional Patent Application Ser. No. 61/162,455, filed on Mar. 23, 2009, which is hereby incorporated by reference in its entirety.

### TECHNICAL FIELD

The compositions and methods relate to lipase/acyltransferase enzymes from prokaryotic and eukaryotic organisms that can be used in such applications as lipid stain removal from fabrics and hard surfaces and in chemical synthesis reactions.

### BACKGROUND

Acyltransferases are enzymes capable of transferring an acyl group from a donor molecule to an acceptor molecule. Such enzymes are assigned the formal Enzyme Classification number 2.3 (EC 2.3). The activity of acyltransferases includes the related but distinct activities of removing an acyl group from a donor molecule, i.e., a “lipolytic” or “lipase” activity, and transferring an acyl group to an acceptor molecule, i.e., a “synthetic” activity. Using suitable donor and acceptor molecules, either or both of these activities can be exploited to achieve a desired result. The terms “lipase,” “acyltransferase,” “transesterase,” and “esterase” are often used to describe the activity that is of interest in a particular enzyme but do not exclude the other activities.

One major industrial use for acyltransferases is to remove oily soil and stains containing triglycerides and fatty acids from fabrics, dishes, and other surfaces. This application relies on the lipase activity of the enzyme, and the acceptor molecule maybe primarily water. The specificity of the acyltransferases, e.g., with respect to donor (substrate) chain-length and charge, determine the types of triglycerides and fatty acids or other substrates that are most efficiently hydrolyzed by the enzyme. For use in cleaning applications, an acyltransferase is typically used in combination with a suitable detergent composition.

The synthetic activity of acyltransferases is of use for acetylating any number of different acceptor molecules to produce esters, including fatty acid and glycerol esters. Exemplary reactions that rely on the acylation or transesterification activity are for the production of pharmaceuticals and biofuels. The specificity of the acyltransferases with respect to donor and acceptor molecules is largely determined by chain-length and charge.

The need exists for new acyltransferases that have useful biochemical features.

### SUMMARY

The present compositions and methods relate to a family of lipases/acyltransferases that share conserved amino acid sequence motifs and have limited homology to extracellular acyltransferases isolated from Candida parasilopsis (i.e., Cpa-L) and Candida albicans (i.e., Cal-L). Based on the phylogenic clustering of the present lipases/acyltransferases with lipase A from Candida Antarctica, they are herein collectively referred to as CalA-related lipases/acyltransferases, which is abbreviated (CALA).

In a first aspect, a recombinant lipase/acyltransferase enzyme having only limited amino acid sequence identity to Candida albicans Cal-L lipase/acyltransferase is provided, comprising:

a) a first amino acid sequence motif GX₁SX₂G at residues corresponding to positions 192-196 of the Cpa-L amino acid sequence (SEQ ID NO: 8), where X₁ is an aromatic amino acid and X₂ is an amino acid selected from the group consisting of G, E, or Q;

b) a second amino acid sequence motif YAX₁X₂X₃, at residues corresponding to positions 210-214 of the Cpa-L amino acid sequence (SEQ ID NO: 8), where X₁ is P or K, X₂ is an acidic amino acid, and X₃ is a non-polar aliphatic amino acid;

c) lipase/esterase activity based on hydrolysis of p-nitrophenylbutyrate in an aqueous solution.

In some embodiments, the lipase/acyltransferase has less than about 50% amino acid sequence identity to Cal-L lipase/acyltransferase having the amino acid sequence of SEQ ID NO: 8.

In some embodiments, the lipase/acyltransferase has a precursor amino acid sequence of at least 390 amino acid residues.

In some embodiments, X₁ in the first amino acid sequence motif is selected from the group consisting of Y and H. In some embodiments, X₂ in the first amino acid sequence motif is selected from the group consisting of G and Q. In particular embodiments, the first amino acid sequence motif has a sequence selected from the group consisting of GYSGG, GYSQG, and GHSQG.

In some embodiments, X₁ in the second amino acid sequence motif is selected from the group consisting of D and E. In some embodiments, X₂ in the second amino acid sequence motif is selected from the group consisting of L, V, and I. In particular embodiments, the second amino acid sequence motif has a sequence selected from the group consisting of YAPEL, YAPDV, YAPDL, YAPEI, and YAKEL.

In some embodiments, the lipase/acyltransferase has an amino acid sequence having at least 90% identity to an amino acid sequence selected from the group consisting of SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53. In particular embodiments, the lipase/acyltransferase does not have the amino acid sequence of SEQ ID NO: 5 or SEQ ID NO: 8.

In some embodiments, the lipase/acyltransferase is selected from the group consisting of Aad-L, Pst-L, Sco-L, Mfu-L, Rsp-L, Cje-L, Ate-L, Aor-L-0488, Afu-L, Ani-L, Acl-L, Aor-L-6767, Fve-L, Fgr-L, Ksp-L, and Dha-L. In particular embodiments, the lipase/acyltransferase is not Cal-L or CpaL.

In a related aspect, a recombinant lipase/acyltransferase enzyme having at least 90% amino acid sequence identity to an amino acid sequence selected from the group consisting of SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53, is provided.

In another aspect, a composition comprising one or more of the above lipase/acyltransferase enzymes is provided. In some embodiments, the lipase/acyltransferase is expressed in a heterologous host cell.

In some embodiments, the composition is a detergent composition. In some embodiments, the composition is a detergent composition and the lipase/acyltransferase enzyme is Sco-L.

In a related aspect, a composition comprising a recombinant lipase/acyltransferase enzyme having at least 90% amino acid sequence identity to an amino acid sequence selected from the group consisting of SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53 is provided.

In another aspect, a method for removing an oily soil or stain from a surface is provided, comprising contacting the surface with a composition comprising one or more of the above lipase/acyltransferase enzymes.

In some embodiments, the composition is a detergent composition. In some embodiments, the composition is a detergent composition and the lipase/acyltransferase is Sco-L. In some embodiments, the surface is a textile surface.

In another aspect, a method for forming a peracid is provided, comprising contacting an acyl donor and hydrogen peroxide with one or more of the lipase/acyltransferase enzymes described above. In some embodiments, the lipase/acyltransferase enzyme is Aad-L.

In another aspect, a method for forming an ester surfactant is provided, comprising contacting an acyl donor and acceptor with one or more of the lipase/acyltransferase enzymes described above. In some embodiments, the lipase/acyltransferase enzyme is Aad-L, Pst-L, Sco-L, or Mfu-L.

In another aspect, a method for making biodiesel or a synthetic lubricant is provided, comprising contacting an acyl donor and acceptor with one or more of the lipase/acyltransferase enzymes described above. In some embodiments, the lipase/acyltransferase enzyme is Aad-L or Pst-L.

In some embodiments, the lipase/acyltransferase enzyme use in the above methods is expressed in a heterologous host cell.

In another aspect, an expression vector is provided, comprising a polynucleotide encoding a lipase/acyltransferase enzyme as described above and a signal sequence to cause secretion of the lipase/acyltransferase enzyme.

In a related aspect, an expression vector is provided, comprising a polynucleotide encoding the lipase/acyltransferase enzyme Cal-L or Cpa-L and a signal sequence to cause secretion of the lipase/acyltransferase enzyme.

In another aspect, a method for expressing a lipase/acyltransferase enzyme is provided, comprising: introducing an expression vector as described into a suitable host, expressing the lipase/acyltransferase enzyme, and recovering the lipase/acyltransferase enzyme expressed.

These and other aspects of CALA compositions and methods will be apparent from the following description and drawings.

### BRIEF DESCRIPTION OF THE DRAWINGS

FIGS. 1A-J show a partial amino acid sequence alignment of CALA amino acid acid sequences.

FIG. 2 is a dendrogram showing the similarity of different CALA to Cpa-L and other known and putative lipase/acyltransferases.

FIG. 3 shows a diagram of a plasmid used to express CALA in Hansenula polymorpha.

FIG. 4 shows a diagram of a plasmid used to express CALA in Streptomyces lividans.

FIG. 5 shows a diagram of a plasmid used to express CALA in Trichoderma reesei.

FIG. 6 is a graph showing the activity of Sco-L at different temperatures.

FIG. 7 is a graph showing the hydrolysis of a pNB substrate by Cal-L, Cpa-L, Aad-L, and Pst-L.

FIG. 8A is a graph showing the hydrolysis of a pNB substrate by Sco-L. FIG. 8B is a graph showing the hydrolysis of a pNB substrate by Cje-L, Rsp-L, and Mfu-L.

FIG. 9 is a graph showing the hydrolysis of a pNPP substrate by Cal-L, Cpa-L, Aad-L, and Pst-L.

FIG. 10A is a graph showing the hydrolysis of a pNPP substrate by Sco-L. FIG. 10B is a graph showing the hydrolysis of a pNPP substrate by Mfu-L.

FIGS. 11A-11C are graphs showing the results of HPLC analysis of transesterification reactions involving Aad-L and Pst-L. FIG. 11A shows a profile of the reference triolein control. FIGS. 11B and 11C show the products of triolein hydrolysis produced by Pst-L and Aad-L. FIG. 11D shows an ethyl oleate standard.

FIG. 12 is a graph showing peracetic acid generation by Aad-L, Pst-L, and Cal-L.

FIGS. 13A and 13B are graphs showing the formation of biodiesel ethyl oleate by Aad-L and appropriate controls.

FIGS. 14A-C show a table identifying the polypeptide and polynucleotide sequences referred to in the description. FIGS. 14D-U show the actual polypeptide and polynucleotide sequences.

### DETAILED DESCRIPTION

### I. Introduction

Described are compositions and methods relating to a family of lipases/acyltransferases collectively referred to as CalA-related lipases/acyltransferases (CALA). CALA share conserved amino acid sequence motifs and have limited homology (i.e., about 18-49%) to extracellular acyltransferases isolated from Candida parasilopsis (i.e., Cpa-L) and Candida albicans (i.e., Cal-L).

Following cloning and expression in suitable organisms, CALA were shown to have lipase and/or acyltransferases activity, in some cases in the presence of detergent compositions, making them useful for a variety of cleaning and synthesis applications.

Various features and applications of CALA are described in detail, below.

### II. Definitions

Unless defined otherwise herein, all technical and scientific terms should be accorded their ordinary meaning as described, for example, in Singleton and Sainsbury, Dictionary of Microbiology and Molecular Biology, 2d Ed., John Wiley and Sons, NY (1994); Hale and Marham, The Harper Collins Dictionary of Biology, Harper Perennial, NY (1991); and Kieser et al., Practical Streptomyces Genetics, the John Innes Foundation, Norwich, United Kingdom (2000). The following terms are defined for clarity:

As used herein, the term “enzyme” refers to a protein that catalyzes a chemical reaction. The catalytic function of an enzyme constitutes its “activity” or “enzymatic activity.” An enzyme can be classified according to the type of catalytic function it performs and assigned an appropriate Enzyme Classification number.

As used herein, the term “substrate” refers to a substance (e.g., a molecule) upon which an enzyme performs its catalytic activity to generate a product. In the case of a lipase/acyltransferase, the substrate is typically the donor molecule.

As used herein, an “acyltransferase is an enzymes capable of transferring an acyl group from a donor molecule to an acceptor molecule and having the enzyme classification EC 2.3. The activity of acyltransferases includes the related but distinct activities of removing an acyl group from a donor molecule, i.e., a “lipolytic” or “lipase” activity, and transferring an acyl group to an acceptor molecule, i.e., a “synthetic” activity. The term “lipase/acyltransferase” is used herein to emphasize the duality of function.

As used herein, the term “acyl” refers to an organic group with the general formula RCO—, which can be derived from an organic acid by removal of the —OH group. As used herein, no limits are placed on the R group except where specified.

As used herein, the term “acylation” refers to a chemical transformation in which one of the substituents of a molecule is substituted by an acyl group, or the process of adding an acyl group to a molecule.

As used herein, a “transferase” is an enzyme that catalyzes the transfer of a functional group from one substrate (a donor) to another substrate (an acceptor).

As used herein, the abbreviation “CALA” is used for convenience and brevity to refer collectively to CalA-related lipases/acyltransferases. Cpa-L and Cal-L are not CALA but may be referred to in their description.

As used herein, the term “polypeptide” refers to a polymeric form of amino acids linked via peptide bonds. The polymer may be linear or branched and may include modified amino acids or be interrupted by non-amino acids. Polypeptides may be glycosylated, phosphorylated, acetylated, prenylated, or otherwise modified, and may include naturally-occurring or synthetic amino acids. The terms “polypeptide” and “protein” are used interchangeably and without distinction. Unless otherwise indicated, amino acid sequences are written left to right in amino to carboxy orientation, using the conventional one-letter or three-letter codes.

As used herein, the term “polynucleotide” refers to a polymeric form of nucleotides of any length and any three-dimensional structure (including linear and circular), which may be single or multi-stranded (e.g., single-stranded, double-stranded, triple-helical, etc.), and which contain deoxyribonucleotides, ribonucleotides, and/or analogs or modified forms, thereof. Polynucleotides include RNA, DNA, and hybrids and derivatives, thereof. A sequence of nucleotides may be interrupted by non-nucleotide components and one or more phosphodiester linkages may be replaced by alternative linking groups. Where a polynucleotide encodes a polypeptide, it will be appreciated that because the genetic code is degenerate, more than one polynucleotide may encode a particular amino acid sequence. Polynucleotides may be naturally occurring or non-naturally occurring. The terms “polynucleotide” and “nucleic acid” and “oligonucleotide” are used interchangeably. Unless otherwise indicated, polynucleotides are written left to right in 5′ to 3′ orientation.

As used herein, the term “primer” refers to an oligonucleotide useful for initiating nucleic acid synthesis (e.g., in a sequencing or PCR reaction) or capable of hybridizing to a target sequence. Primers are typically from about 10 to about 80 nucleotides in length, and may be 15-40 nucleotides in length.

As used herein, the terms “wild-type,” “native,” and “naturally-occurring” refer to polypeptides or polynucleotides that are found in nature.

As used herein, a “variant” protein differ from the “parent” protein from which it is derived by the substitution, deletion, or addition of a small number of amino acid residues, for example, 1, 2, 3, 4, 5, 6, 7, 8, 9, 10, 11, 12, 13, 14, 15, 16, 17, 18, 19, 20, or more amino acid residues. In some cases, the parent protein is a “wild-type,” “native,” or “naturally-occurring” polypeptides. Variant proteins may be described as having a certain percentage sequence identity with a parent protein, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at even at least 99%, which can be determined using any suitable software program known in the art, for example those described in CURRENT PROTOCOLS IN MOLECULAR BIOLOGY (F. M. Ausubel et al. (eds) 1987, Supplement 30, section 7.7.18).

Preferred programs include the Vector NTI Advance™ 9.0 (Invitrogen Corp. Carlsbad, Calif.), GCG Pileup program, FASTA (Pearson et al. (1988) Proc. Natl, Acad. Sci. USA 85:2444-2448), and BLAST (BLAST Manual, Altschul et al., Natl Cent. Biotechnol. Inf., Natl Lib. Med. (NCIB NLM NIH), Bethesda, Md., and Altschul et al. (1997) NAR 25:3389-3402). Another preferred alignment program is ALIGN Plus (Scientific and Educational Software, PA), preferably using default parameters. Another sequence software program that finds use is the TFASTA Data Searching Program available in the Sequence Software Package Version 6.0 (Genetics Computer Group, University of Wisconsin, Madison, Wis.).

As used herein, the term “analogous polypeptide sequence” and similar terms, refers to a polypeptide that shares structural and/or functional features with a reference polypeptide.

As used herein, the term “homologous polypeptide” refers to a polypeptide that shares structural features, particularly amino acid sequence identity, with a reference polypeptide. No distinction is made between homology and identity.

As used herein, the term “remaining amino acid sequence” refers to amino acid sequences in a polypeptide other than those specified. For example, where a polypeptide is specified to have one or more conserved amino acid sequences motifs, the remaining amino acid sequence are those other than the amino acid sequences in the conserved motif(s).

As used herein, the term “limited amino acid sequence identity (homology)” means that a subject amino acid sequence is minimally related to another sequence such that it would not be considered a variant, homolog, or related sequence based on conventional similarity searches, e.g., primary structure alignments. For example, a polypeptide having less than about 50% amino acid sequence identity to a reference polypeptide is considered as having limited amino acid sequence identity to the reference polypeptide.

As used herein, an “expression vector” is a DNA construct containing a DNA coding sequence (e.g., a gene sequence) that is operably-linked to one or more suitable control sequence(s) capable of effecting expression of the coding sequence in a host. Such control sequences include promoters, terminators, enhancers, and the like. The DNA construct may be a plasmid, a phage particle, a PCR product, or other linear DNA.

As used herein, the term “expression” refers to the process by which a polypeptide is produced based on the nucleic acid sequence of a gene. The process includes both transcription, translation, and, optionally secretion.

As used herein, a “host cell” is a cell or cell line into which a recombinant expression vector is introduced for production of a polypeptide or for propagating a nucleic acid encoding a polypeptide. Host cells include progeny of a single host cell, which progeny may not be completely identical (in morphology or in total genomic DNA complement) to the original parent cell due to natural, accidental, or deliberate mutation. A host cell may be bacterial, fungal, plant, or animal.

As used herein, the term “introduced,” in the context of inserting a nucleic acid sequence into a cell, includes the processes of “transfection,” “transformation,” and “transduction,” and refers to the incorporation or insertion of a nucleic acid sequence into a eukaryotic or prokaryotic cell.

As used herein, the term “recovered,” “isolated,” “purified,” and “separated” refer to a material (e.g., a protein, nucleic acid, or cell) that is removed from at least one component with which it is naturally associated. For example, these terms may refer to a material which is substantially or essentially free from components which normally accompany it as found in its native state, such as an intact biological system or substantially or essentially free from components associated with its heterologous expression in a host organism.

As used herein, “cleaning compositions” and “cleaning formulations” refer to compositions, i.e., admixtures of ingredients, that find use in the removal of undesired soil and stains from items to be cleaned, such as fabric, dishes, contact lenses, skin, hair, teeth, and other surfaces. The specific selection of cleaning composition materials depend on the surface, item, or fabric to be cleaned, the desired form of the composition, and the enzymes present.

As used herein, the terms “detergent composition” and “detergent formulation” are used in reference to admixtures of ingredients which are intended for use in a wash medium for the cleaning of soiled or stained objects. Detergent compositions encompass cleaning compositions but require the presence of at least one surfactant.

As used herein, a “dishwashing composition” is a composition for cleaning dishes, including but not limited to granular and liquid forms.

As used herein, a “fabric” is a textile material, including cloths, yarns, and fibers. Fabric may be woven or non-woven and may be from natural or synthetic materials.

As used herein, a “fabric cleaning composition” is a cleaning composition suitable for cleaning fabrics, including but not limited to, granular, liquid and bar forms.

As used herein, the phrase “detergent stability” refers to the ability of a subject molecule, such as an enzyme, to retain activity in a detergent composition.

As used herein, the phrase, “stability to proteolysis” refers to the ability of a protein (e.g., an enzyme) to avoid proteolysis, e.g., when suspended or dissolved in a cleaning composition.

As used herein, the term “disinfecting” refers to the removal or destruction of organisms (e.g., microbes) from a surface.

As used herein, the terms “contacting” and “exposing” refer to placing at least one enzyme in sufficient proximity to its cognate substrate to enable the enzyme to convert the substrate to at least one end-product. The end-product may be a “product of interest” (i.e., an end-product that is the desired outcome of the fermentation reaction). “Contacting” includes mixing a solution comprising an enzyme with the cognate substrate.

As used herein, an “aqueous medium” is a solution or mixed solution/suspension in which the solvent is primarily water. An aqueous medium is substantially free of inorganic solvents but may include surfactants, salts, buffers, substrates, builders, chelating agents, and the like.

As used herein, “perhydrolase activity” is the ability to catalyze a perhydrolysis reaction that results in the production of peracids.

As used herein, the term “peracid” refers to a molecule having the general formula RC(═O)OOH. Peracids may be derived from a carboxylic acid ester that has been reacted with hydrogen peroxide to form a highly reactive product. Peracids are powerful oxidants.

As used herein, the singular terms “a,” “an,” and “the” includes the plural unless the context clearly indicates otherwise. Thus, for example, reference to a composition containing “a compound” includes a mixture of two or more compounds. The term “or” generally means “and/or,” unless the content clearly dictates otherwise.

Headings are provided for convenience, and a description provided under one heading may apply equally to other parts of the disclosure. All recited species and ranges can be expressly included or excluded by suitable language or provisos.

Numeric ranges are inclusive of the numbers defining the range. Where a range of values is provided, it is understood that each intervening value between the upper and lower limits of that range is also specifically disclosed, to a tenth of the unit of the lower limit (unless the context clearly dictates otherwise). The upper and lower limits of smaller ranges may independently be included or excluded in the range.

The following abbreviations/acronyms have the following meanings unless otherwise specified: EC=enzyme commission; kDa=kiloDalton; MW=molecular weight; w/v=weight/volume; w/w=weight/weight; v/v=volume/volume; wt %=weight percent; ° C.=degrees Centigrade; H₂O=water; H₂O₂=hydrogen peroxide; dH₂O or DI=deionized water; dIH₂O=deionized water, Milli-Q filtration; g or gm=gram; μg=microgram; mg=milligram; kg=kilogram; μL and μl=microliter; mL and ml=milliliter; mm=millimeter; nm=nanometer; μm=micrometer; M=molar; mM=millimolar; μM=micromolar; U=unit; ppm=parts per million; sec and ″=second; min and ′=minute; hr=hour; gpg=grains per gallon; rpm=revolutions per minute; bp=base pair; kb=kilobase; kV=kiloVolt; pF=microFarad; Ω=Ohm; EtOH=ethanol; eq.=equivalent; N=normal; CI=Colour (Color) Index; CAS=Chemical Abstracts Society; PVA=poly(vinyl) alcohol; DMSO=dimethyl sulfoxide; NEFA=non-esterified fatty acid; DTT=dithiothreitol; HEPES=N-2-hydroxyethylpiperazine-N′-2-ethanesulfonic acid; MOPS=3-(N-morpholino)propanesulfonic acid; TES=2-{[tris(hydroxymethyl)methyl]amino}ethanesulfonic acid; ABTS=2,2′-azino-bis(3-ethylbenzothiazoline-6-sulfonic acid); pNB=para-nitrophenyl butyrate; pNPP=para-nitrophenyl palmitate; pNO=para-nitrophenyl octanoate; pND=para-nitrophenyl decanoate; pNP=para-nitrophenyl palmitate; pNS=para-nitrophenyl stearate; YPD or YEPD=yeast extract peptone dextrose; PDA=potato dextrose agar; UFC=ultrafiltered concentrate; TLC=thin layer chromatography; HPTLC=high performance thin layer chromatography; HPLC=high performance liquid chromatography; LC/MS CAD=liquid chromatography coupled to mass spectrometry, charged aerosol detections; APCI=atmospheric pressure chemical ionization; ×g=times gravity.

All patents, patent applications, articles and publications mentioned herein, both supra and infra, are hereby expressly incorporated herein by reference.

### III. CALA Polypeptides and Polynucleotides

A. CALA Polypeptides

One aspect of the present compositions and methods includes CalA-related lipases/acyltransferases (CALA). CALA are a family of eukaryotic and prokaryotic lipases/acyltransferases that share limited homology (e.g., about 18-49%) to known extracellular acyltransferases isolated from Candida parasilopsis (i.e., Cpa-L) and Candida albicans (i.e., Cal-L). The identification of CALA, their homology to Cpa-L and Cal-L, and their homology to each other, are described in detail in Example 2, including Table 1. Because of their limited sequence homology to Cpa-L and Cal-L, CALA would not be identified in routine sequence searches for Cpa-L or Cal-L homologs. As evidence of their unknown function, the amino acid sequences now identified as CALA were previously annotated as undefined or poorly characterized hypothetical proteins that were either not known to be lipases and/or acyltransferases, or were suspected to be lipases but had no known acyltransferase activity.

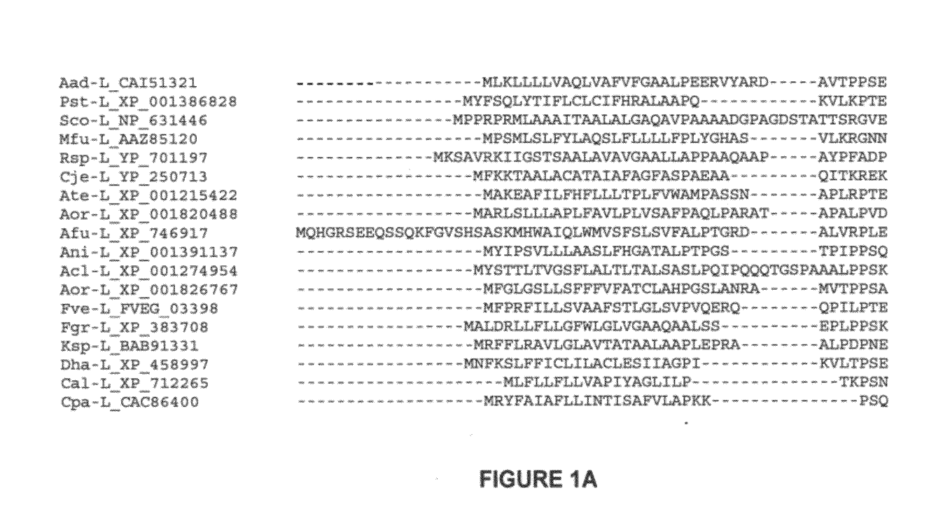

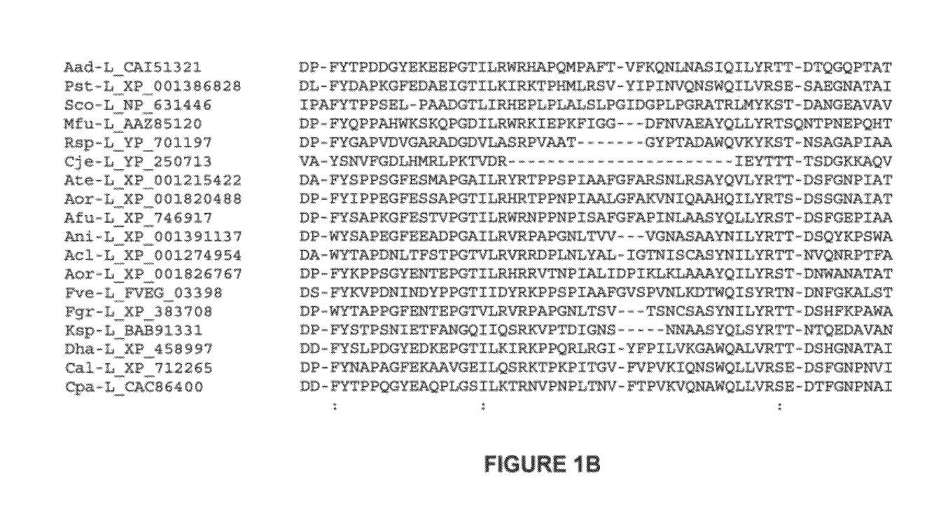

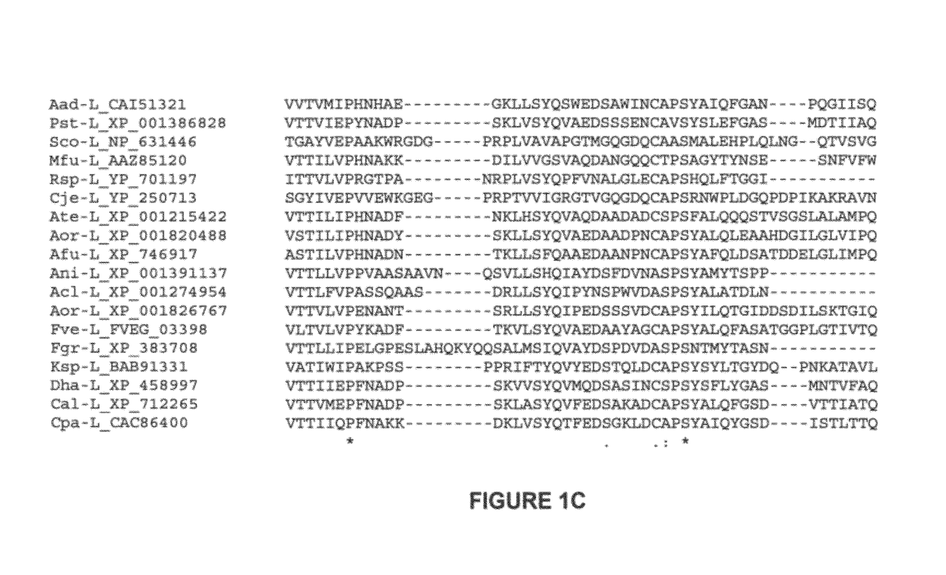

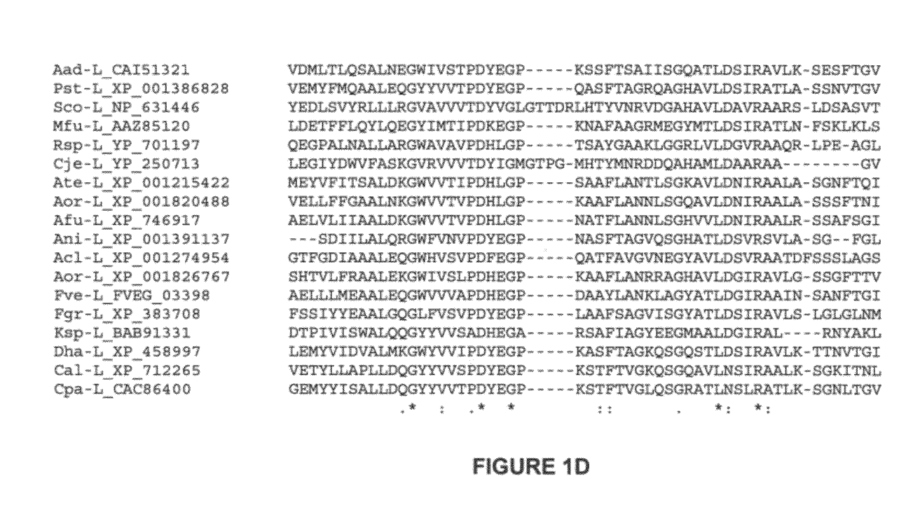

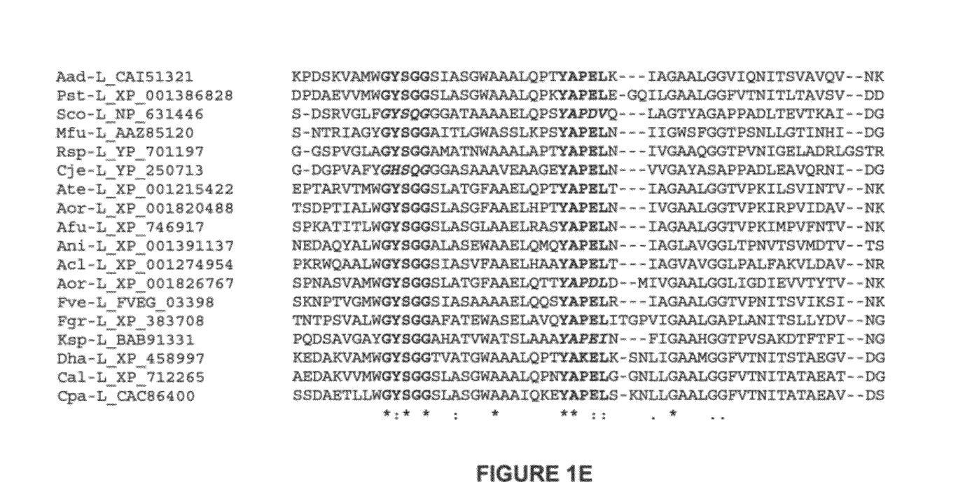

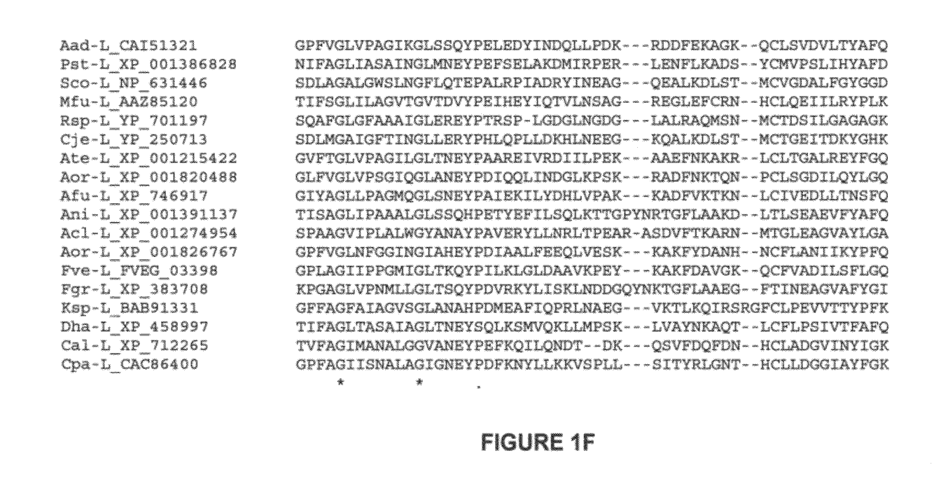

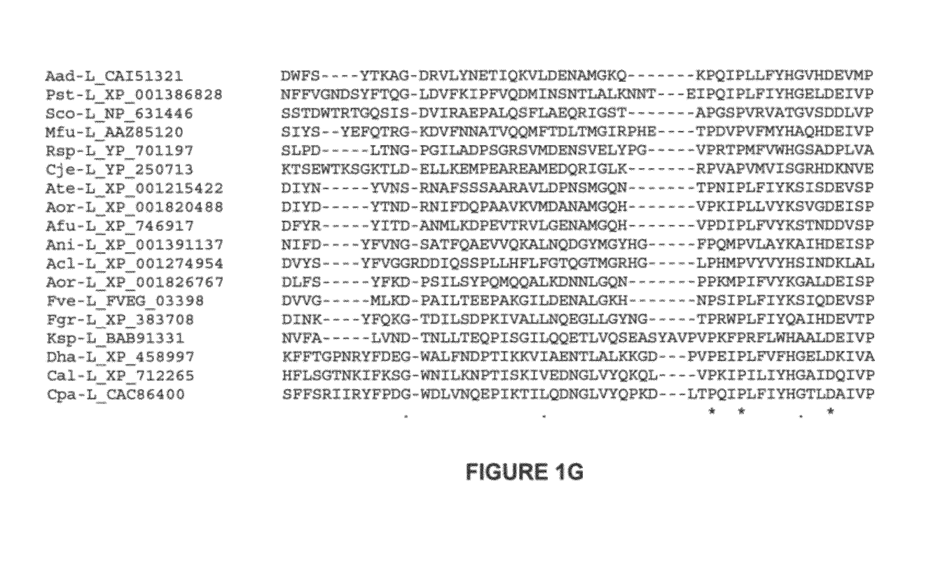

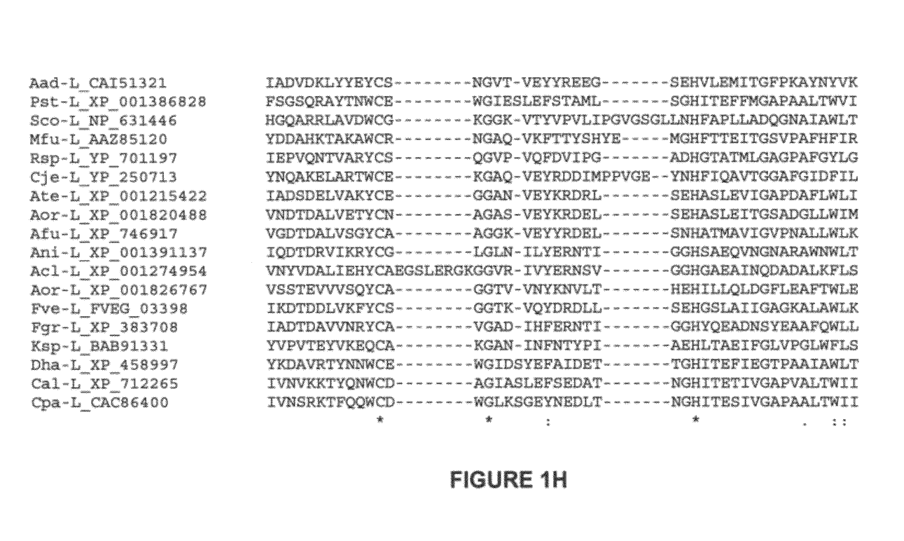

While the present CALA are found in a variety of different organisms, they share distinct structural features with respect to their primary amino acid sequence. For convenience, these structural features are described with reference to the amino acid sequence of Cpa-L, as found in Genbank Accession No. XP_{—}712265 (gi68487709; SEQ ID NO: 7). An alignment of different CALA with Cpa-L and Cal-L is shown in FIGS. 1A-J and serves as the basis for describing structurally conserved features.

Both eukaryotic and prokaryotic CALA share a first common conserved consensus amino acid motif, GYSGG, at the residues of each CALA corresponding to residues 192-196 of the Cpa-L amino acid sequence. Most CALA have the exact sequence, GYSGG, with the exception of Sco-L, which has the sequence GYSQG, and CjeL, which has the sequence GHSQG. Another amino acid sequences that was identified in the initial screen for CALA had the sequence GYSEG (not shown). Therefore, this conserved sequence motif can be generalized as GX₁SX₂G, where X₁ is an aromatic amino acid, such as Y or H, and X₂ is an amino acid selected from the group consisting of G, E, or Q, as exemplified by G or Q. Note that according to conventional single-letter amino acid nomenclature, E and Q can be referred to collectively as Z.

Both eukaryotic and prokaryotic CALA share a second common conserved consensus amino acid motif, YAPEL, at the residues of each CALA corresponding to residues 210-214 of the Cpa-L amino acid sequence. Most of the present CALA, including all the prokaryotic CALA, have the exact sequence YAPEL, with the exception of Sco-L, which has the sequence YAPDV, Aor-L, which has the sequence YAPDL, and KSP-L, which has the sequence YAPEI. The CALA Dha-L has the sequence YAKEL. Therefore, this conserved sequence motif can be generalized as YAX₁X₂X₃, where X₁ is generally P but can also be K, X₂ is an acidic amino acid, such as D or E, and X₃ is a non-polar aliphatic amino acid selected from the group consisting of L, V, or I.

Another feature of CALA is that they having molecular weights higher than those of typical fungal lipases. In particular, CALA are at least 390 amino acid residues (including the signal peptide) to greater than 400 amino acids in length, with deduced molecular weights of at least 39 kDa. Glycosylation sites are present in the amino acid sequences of CALA from eukaryotes, which may further increase the molecular weights of these enzymes. In contrast, most lipases described in the literature and in the patent databases have shorter polypeptide chains and molecular weights of less than 39 kDa. As examples, lipase 3 of Aspergillus tubigenesis is only 297 amino acid residues in length with a molecular weight of about 30 kDa (which can vary due to degree of glycosylation; U.S. Pat. No. 6,852,346) and the commercial detergent enzyme, LIPEX™ (Novozymes) from Humicola lanuginosus is only 269 amino acids in length (mature protein).

In view of these and other conserved structural features, the CALA can be divided into one or more of several different subgroups, which constitute related but distinguishable embodiments of the present compositions and methods.

In one embodiment, CALA polypeptides include amino acid sequences include either the first conserved sequence motif GX₁SX₂G, the second conserved sequence motif YAX₁X₂X₃, or both. In some embodiments the first sequence motif is GX₁SZG. In particular embodiments, the first sequence motif is selected from the group consisting of GYSGG or GYSQG. In some embodiments the second sequence motif is YAPEL, YAPDV, YAPDL, YAPEI, or YAKEL. In some embodiments, CALA are at least 390 amino acids in length. In particular embodiments, variant CALA have a first sequence motif is selected from the group consisting of GYSGG or GYSQG and the second sequence motif is YAPEL, YAPDV, YAPDL, YAPEI, or YAKEL.

In some embodiments, the CALA are from eukaryotic organisms, exemplified by filamentous fungi, such as Aspergillus spp., Fusarium spp., and yeasts such as Debaryomyces sp., Arxula sp. a Pichia sp., a Kurtzmanomyces sp., and a Malassezia sp. Exemplary CALA from eukaryotic organisms are Aad-L, Pst-L, Mfu-L, Ate-L, AorL-0488, Afu-L, Ani-L, Acl-L, Aor-L-6767, Fve-L, Fgr-L, Ksp-L, and Dha-L. In other embodiments, the CALA are from prokaryotic organisms, exemplified by gram(+) bacteria, such as a Streptomyces sp., a Rhodococcus sp., and a Corynebacterium sp. Exemplary CALA from prokaryotic organisms are Sco-L, Rsp-L, and Cje-L. The conserved amino acid sequence domains can also be used to screen metagenomic libraries, such as the Microbiome Metagenome Database (JGI-DOE, USA) to identify additional CALA.

In further embodiments, the CALA polypeptide is a variant that include one or both of the aforementioned conserved sequence motifs, i.e., GX₁SX₂G and YAX₁X₂X₃ and wherein the remaining amino acid sequence (i.e., other than the conserved motifs) has at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to one or more of the foregoing CALA, for example the Aad-L, Pst-L, Sco-L, Mfu-L, Rsp-L, Cje-L, Ate-L, Aor-L-0488, Afu-L, Ani-L, Acl-L, Aor-L-6767, Fve-L, Fgr-L, Ksp-L, and Dha-L.

In some embodiments, the CALA include one or both of the aforementioned conserved sequence motifs, i.e., GX₁SX₂G and YAX₁X₂X₃ and the remaining amino acid sequences have at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, or SEQ ID NO: 53.

Additional CALA can be identified by searching databases for polypeptides that include the aformentioned first and second sequence motifs. The CALA may be from a eukaryotic organism or from a prokaryotic organism. In some embodiments, the variant CALA is at least 390 amino acids (including the signal peptide), and even at least 400 amino acids, in length. Such variants preferably have lipase and/or acyltransferase activity, which can readily be determined, e.g., using the assays described, herein.

In further embodiments, the CALA are variants of the exemplified CALA that include substitutions, insertions, or deletions that do not substantially affect lipase and/or acyltransferase function, or add advantageous features to the enzymes. In some embodiments, the substitutions, insertions, or deletions are not in the conserved sequence motifs but are instead limited to amino acid sequences outside the conserved motifs. Exemplary substitutions are conservative substitutions, which preserve charge, hydrophobicity, or side group size relative to the parent amino acid sequence. Examples of conservative substitutions are provided in the following Table:

| Original Amino | | |
|---|---|---|
| Acid Residue | Code | Acceptable Substitutions |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

It will be apparent that naturally occurring amino acids can be introduced into a polypeptide by changing the coding sequence of the nucleic acid encoding the polypeptide, while non-naturally-occurring amino acids are typically produced by chemically modifying an expressed polypeptide.

In another embodiment, the CALA has the amino acid sequence of any of the CALA described, herein, with the exceptions that one of both of the conserved motifs include substitutions that are consistent with the first and second sequence motifs, i.e., GX₁SX₂G and YAX₁X₂X₃. For example, a CALA polypeptides having the first conserved sequence motif, GYSGG, can be modified to have the sequence GYSQG, GHSQG, or GYSEG. Similarly, a CALA having the first conserved consensus motif GYSQG, GHSQG, or GYSEG, can be modified to have the consensus sequence, GYSGG. Moreover, a CALA having any of the motif sequences GYSQG, GHSQG, or GYSEG, can be modified to have any one of the other sequences. In another example, a CALA having a second conserved motif with the sequence YAPDV, YAPDL, YAPEI, or YAKEL, can be modified to have the consensus second motif sequence, YAPEL. Similarly, a CALA having the second conserved consensus motif sequence, YAPEL, can be modified to have the sequence YAPDV, YAPDL, YAPEI, or YAKEL. Moreover, a CALA having any of the motif sequences YAPDV, YAPDL, YAPEI, or YAKEL, can be modified to have any one of the other sequences.

Further substitution in the first and second conserved motifs includes conservative amino acid substitutions, as described, above. In yet further embodiments, these substitutions in the conserved motifs are combined with substitutions, insertions, or deletions in the remaining amino acid sequences.

In a further embodiment, a fragment of a CALA polypeptide is provided that retains the lipase and/or acyltransferase activity of the parent polypeptides, which can be determined using, e.g., the assays described herein. Preferred fragments include at least one of the conserved sequence motifs along with the enzyme active site. Also contemplated are chimeric CALA that include a first portion of one CALA and a second portion of another CALA, Cpa-L, Cal-L, or other lipases/acyltransferases.

While CALA are mainly described with reference to mature polypeptides sequences, it will be appreciated that many polypeptides are produced in an immature forms that include additional amino acid sequence that are processed (i.e., cleaved) to yield a mature polypeptide. These full length polypeptides are encompassed by the present compositions and methods, although the mature forms of CALA are generally of the greatest interest in terms of commercial products.

B. CALA Polynucleotides

Another aspect of the present compositions and methods is polynucleotides that encode CALA polypeptides, as described herein. Such polynucleotides include genes isolated from eukaryotic organisms, genes isolated from prokaryotic organisms, and synthetic genes optimized for expression in a heterologous prokaryotic or eukaryotic host organism. Due to the degeneracy of the genetic code, it will be recognized that multiple polynucleotides can encode the same polypeptide.

The polynucleotides may encode variant CALA polypeptides that include substitutions, insertions, or deletions in the conserved sequence motifs, in the remaining amino acid sequences, or both. Variant polynucleotides may also encode chimeric CALA polypeptides or CALA polypeptide fragments.

In some embodiments, variant polynucleotides have a preselected degree of nucleotide sequence identity to a CALA-encoding polynucleotide, such as at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to SEQ ID NO: 3, SEQ ID NO: 12, SEQ ID NO: 15, SEQ ID NO: 18, SEQ ID NO: 21, SEQ ID NO: 24, SEQ ID NO: 28, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, or SEQ ID NO: 55. In particular embodiments, variant polynucleotides have a preselected degree of nucleotide sequence identity to a plurality of CALA-encoding polynucleotides.

In further embodiments, variant polynucleotides hybridize to one or more of the above-described polynucleotides under defined hybridization conditions. For example, variant polynucleotides may hybridize to one or more of the above-described polynucleotides under stringent hybridization conditions, defined as 50° C. and 0.2×SSC (1×SSC=0.15 M NaCl, 0.015 M Na₃ citrate, pH 7.0), or highly stringent conditions, defined as 65° C. and 0.1×SSC (1×SSC=0.15 M NaCl, 0.015 M Na₃ citrate, pH 7.0). These hybridizations are for reference and equivalent stringent and highly stringent conditions can be established using, e.g., different hybridization buffers.

Also provided are vectors comprising polynucleotides encoding CALA. Any vector suitable for propagating a polynucleotide, manipulating a polynucleotide sequence, or expressing a polypeptide encoded by a polynucleotide in a host cell is contemplated. Examples of suitable vectors are provided in standard biotechnology manuals and texts, e.g., Sambrook et al., MOLECULAR CLONING: A LABORATORY MANUAL, 3^{rd} ed., Cold Spring Harbor Laboratory Press, Cold Spring Harbor, N.Y. (2001).

It will be appreciated the vectors may include any number of control elements, such as promotors, enhancers, and terminators and cloning features, such as polylinkers, selectable markers, and the like. Examples of suitable promoters for directing the transcription of CALA, especially in a bacterial host, are the promoter of the lac operon of E. coli, the Streptomyces coelicolor agarase gene dagA promoters, the promoters of the Bacillus licheniformis α-amylase gene (amyL), the promoters of the Geobacillus stearothermophilus maltogenic amylase gene (amyM), the promoters of the Bacillus amyloliquefaciens α-amylase (amyQ), the promoters of the Bacillus subtilis xylA and xylB genes etc. Examples of useful promoters for transcription of CALA in a fungal host are those derived from the gene encoding A. oryzae TAKA amylase, Rhizomucor miehei aspartic proteinase, A. niger neutral α-amylase, A. niger acid stable α-amylase, A. niger glucoamylase, Rhizomucor miehei lipase, A. oryzae alkaline protease, A. oryzae triose phosphate isomerase or A. nidulans acetamidase.

The expression vector may also comprise a suitable transcription terminator and polyadenylation sequences operably connected to a nucleic acid encoding a CALA. Termination and polyadenylation sequences may suitably be derived from the same or different source as the promoter.

The vector may further include a DNA sequence enabling the vector to replicate in the host cell in question. Examples of such sequences are the origins of replication of plasmids pUC19, pACYC177, pUB110, pE194, pAMB1, and pIJ702.

The vector may also comprise a selectable marker, e.g. a gene the product of which complements a defect in the host cell, such as the dal genes from B. subtilis or B. licheniformis, or a gene that confers antibiotic resistance such as ampicillin, kanamycin, chloramphenicol or tetracyclin resistance. Furthermore, the vector may comprise Aspergillus selection markers such as amdS, argB, niaD and sC, a marker giving rise to hygromycin resistance, or the selection may be accomplished by co-transformation, e.g., as described in WO 91/17243.

Expression vectors may remain as episomal nucleic acids suitable for transient expression or may be integrated into the host chromosome for stable expression. Vectors may be tailored for expressing CALA polypeptides in a particular host cell, typically in a microbial cell, such as a bacterial cell, a yeast cell, a filamentous fungus cell, or a plant cell. Vectors may also include heterologous signal sequences to affect the secretion of CALA polypeptides. The procedures used to ligate a nucleic acid encoding a CALA, the promoter, terminator and other elements, respectively, and to insert them into suitable vectors containing the information necessary for replication, are well known (cf., for instance, Sambrook et al., Molecular Cloning: A Laboratory Manual, 2nd Ed., Cold Spring Harbor, 1989).

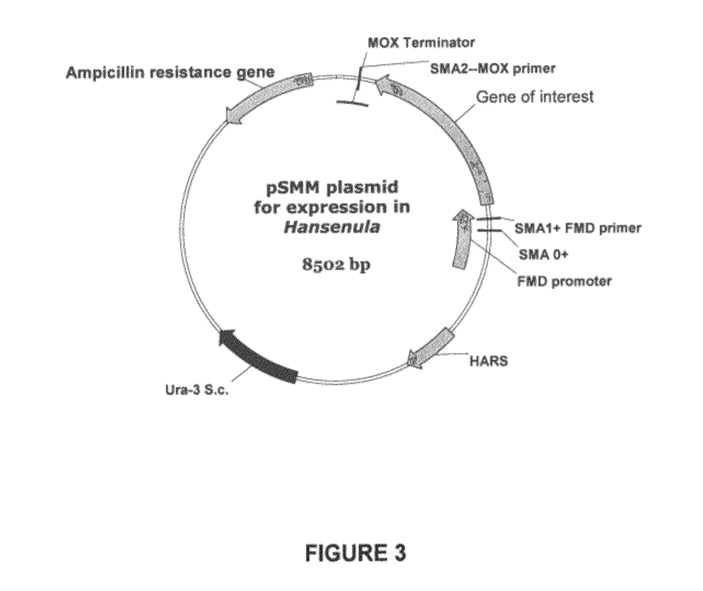

Exemplary expression vectors are described in detail in Examples 3, 4, and 5, with reference to FIGS. 3, 4, and 5, respectively. Also provided are microbial cells, including yeast, fungi, or bacterial cells, comprising a vector that includes a polynucleotide encoding a CALA polypeptide.

### IV. Expression of CALA Polypeptides

Another aspect of the present compositions and methods is expression of CALA polypeptides in a heterologous organism, including microbial cells, such as bacterial cells, yeast cells, filamentous fungus cells, or plant cells. CALA can also be expressed in other eukaryotic cells, such as mammalian cells, although the expense and inconvenience of working with these may make this less desirable.

Examples of bacteria suitable for CALA expression area Gram(+) bacteria such as Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans, or Streptomyces murinus, and Gram(−)bacteria such as E. coli. Examples of yeast are Saccharomyces spp. or Schizosaccharomyces spp. e.g. Saccharomyces cerevisiae. Examples of filamentous fungus are Aspergillus spp., e.g., Aspergillus oryzae or Aspergillus niger. Methods from transforming nucleic acids into these organisms are well known in the art. A suitable procedure for transformation of Aspergillus host cells is described in EP 238 023.

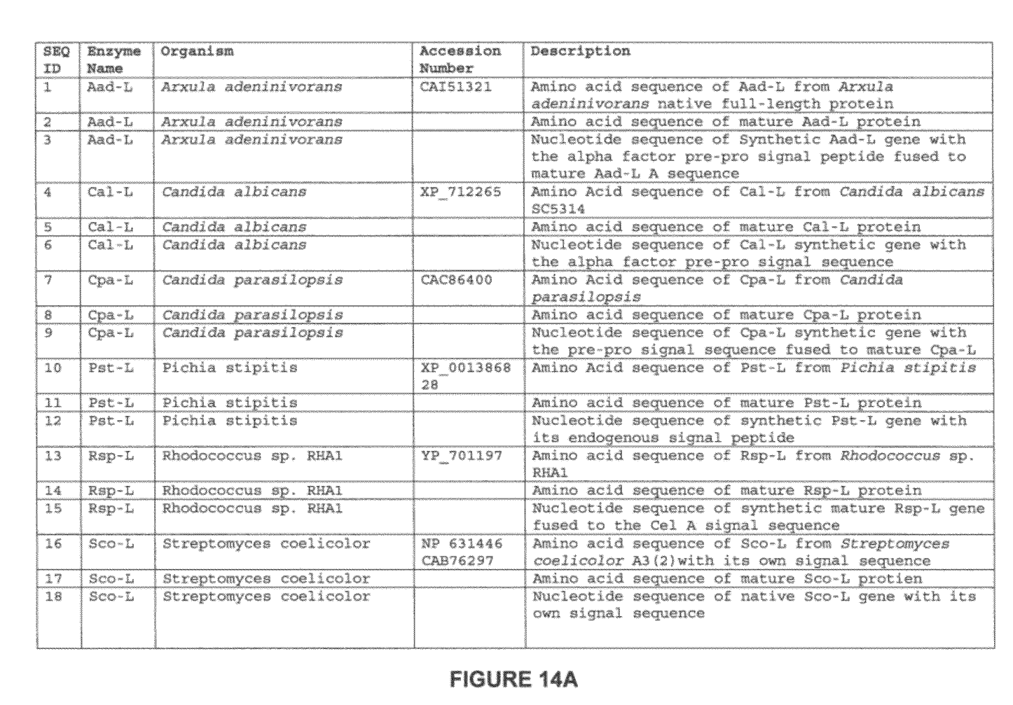

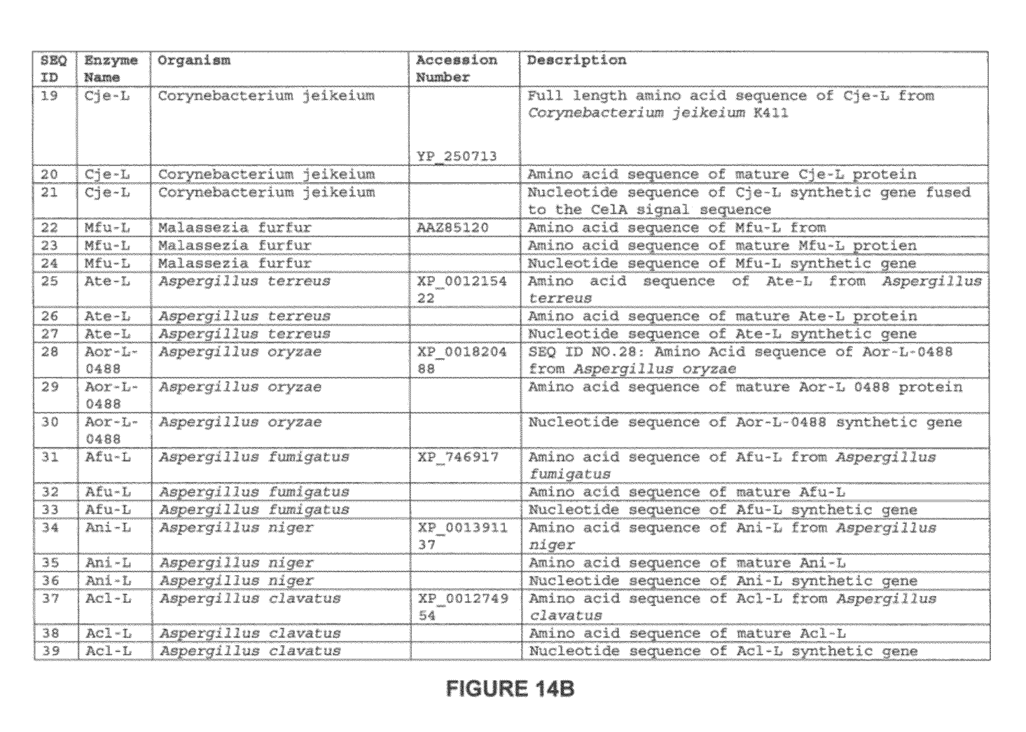

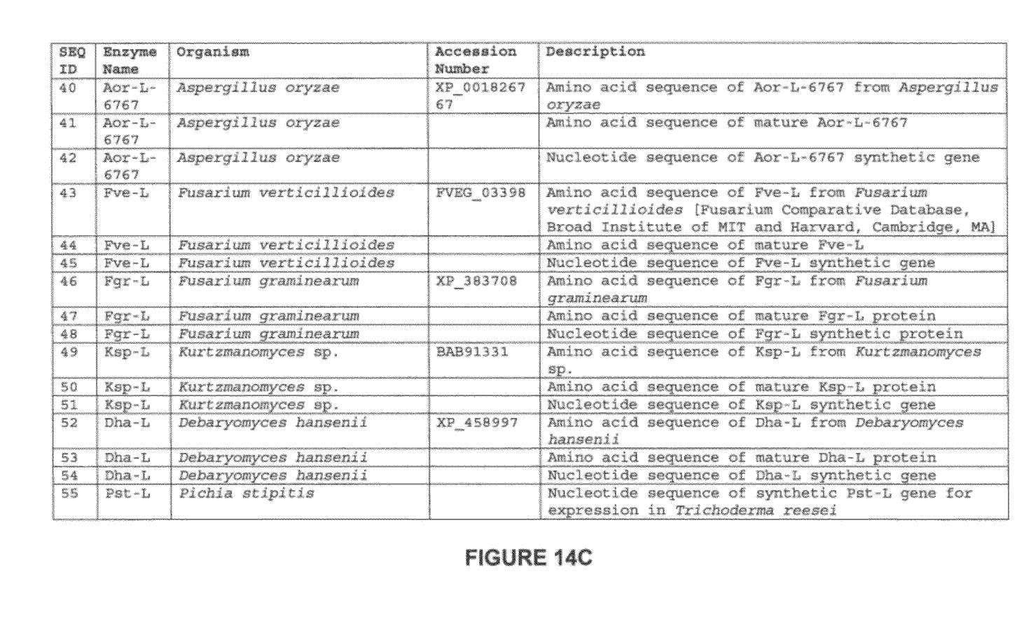

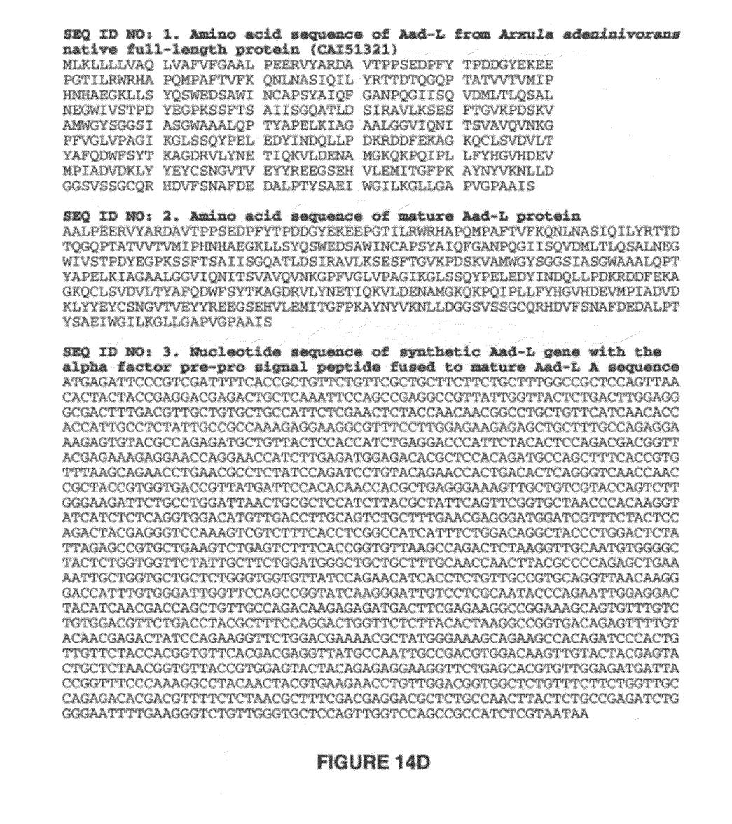

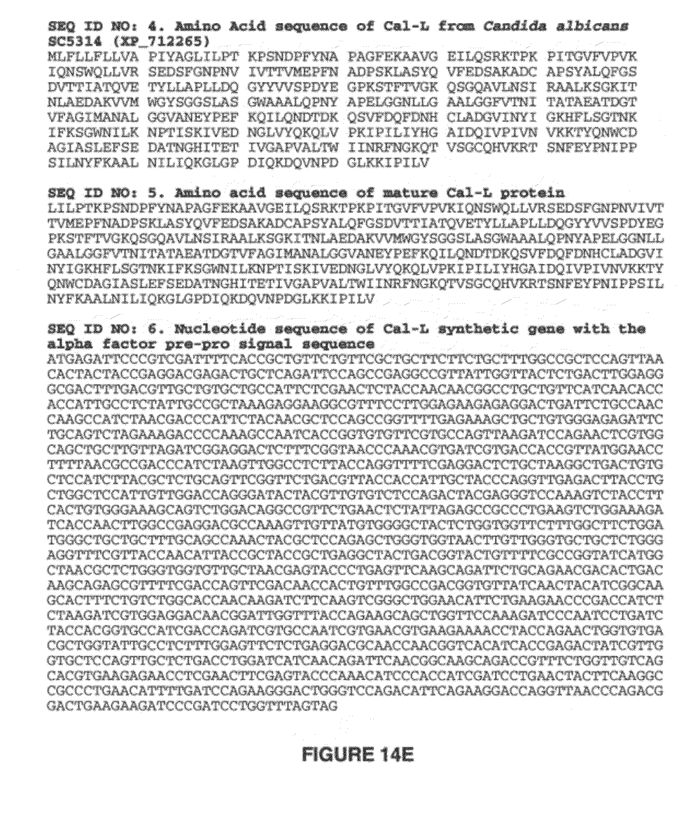

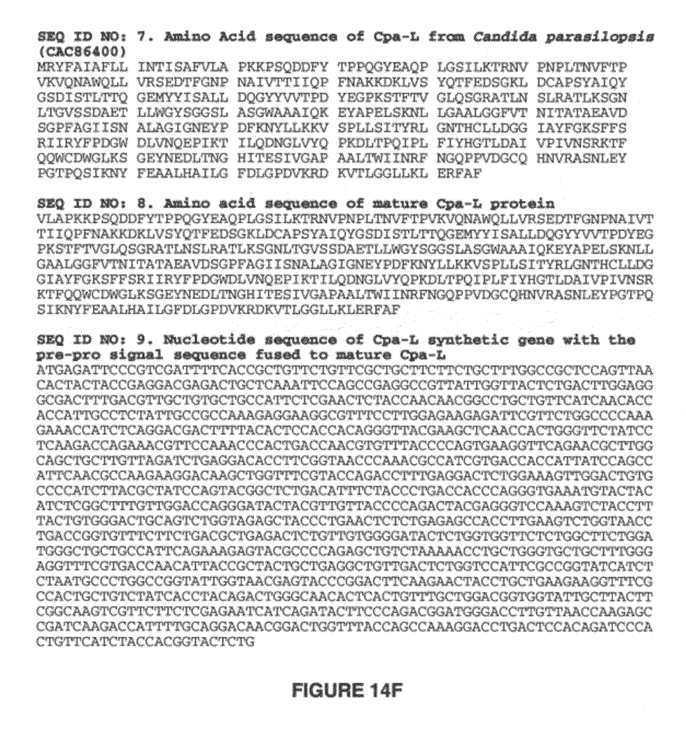

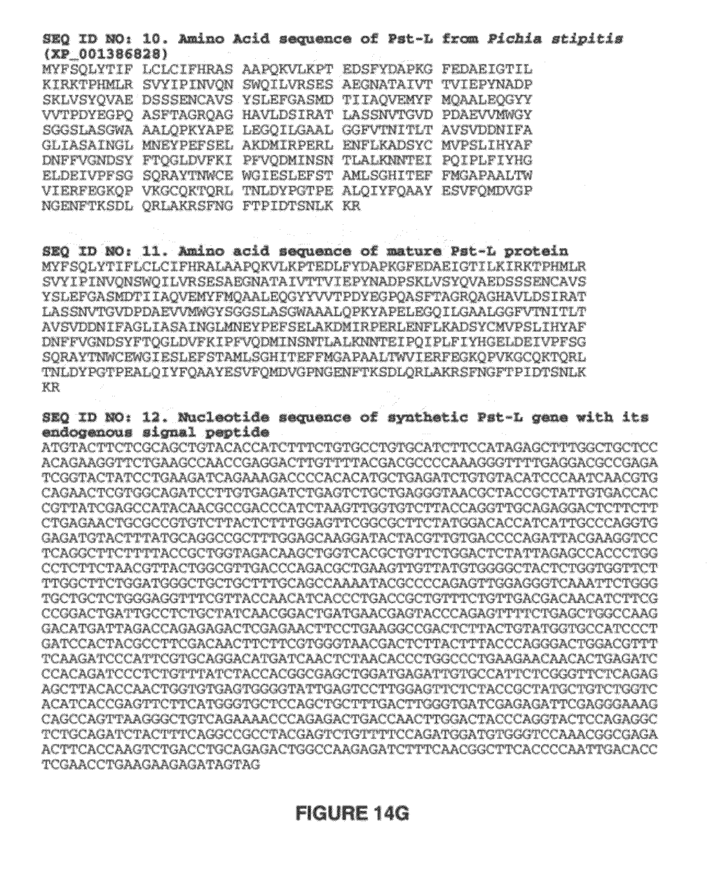

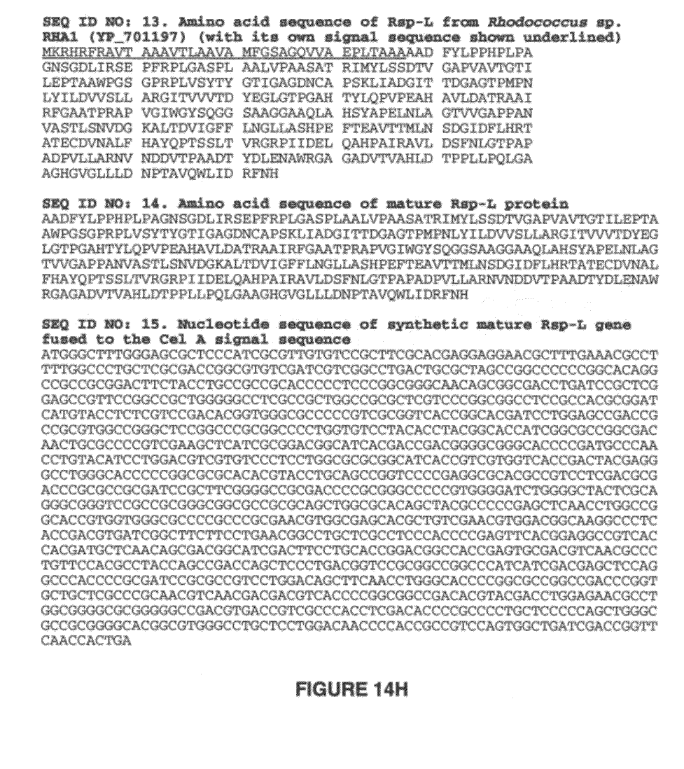

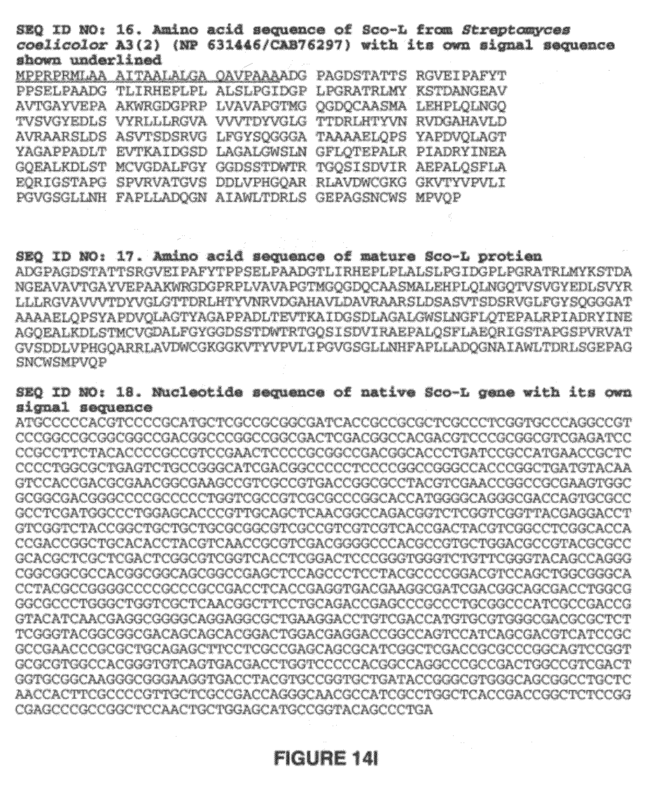

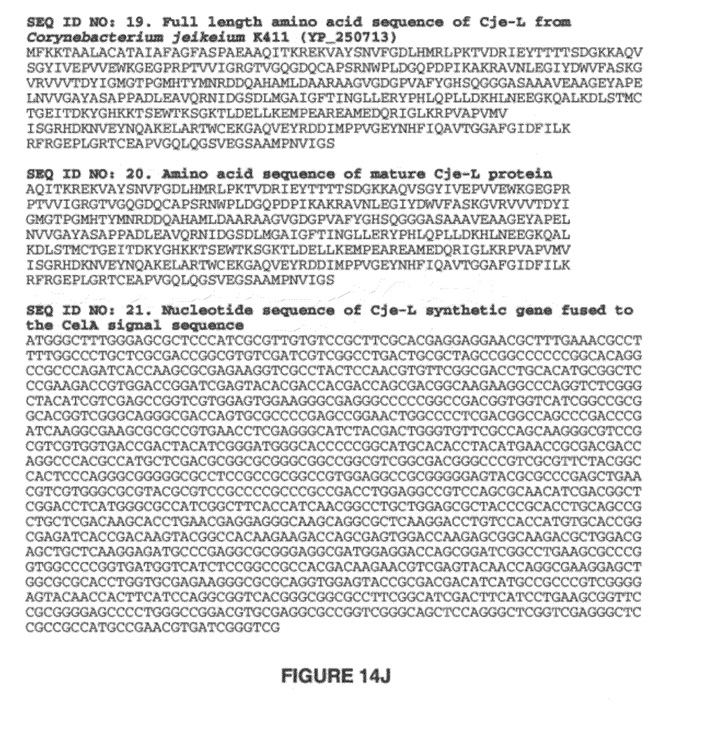

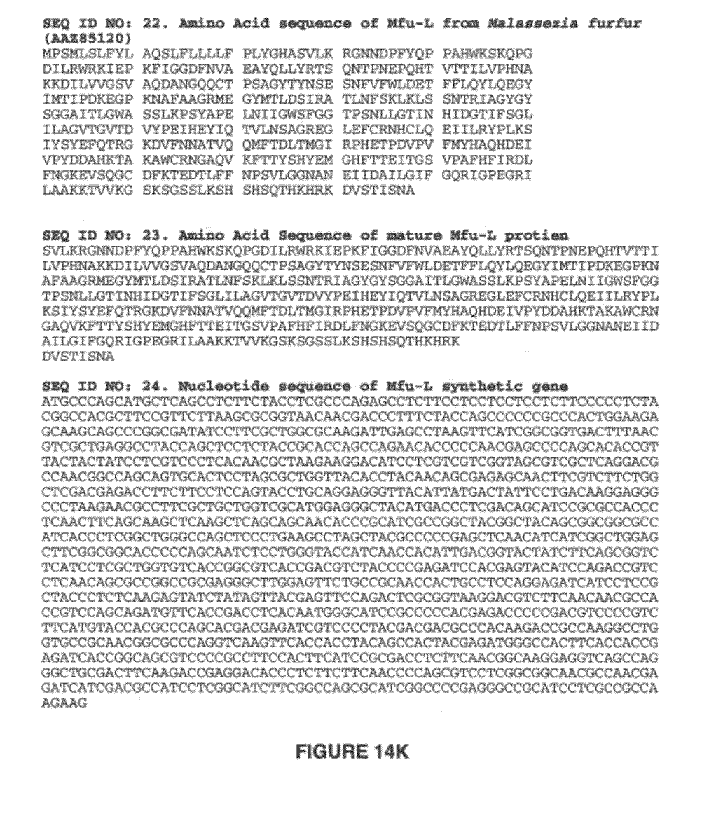

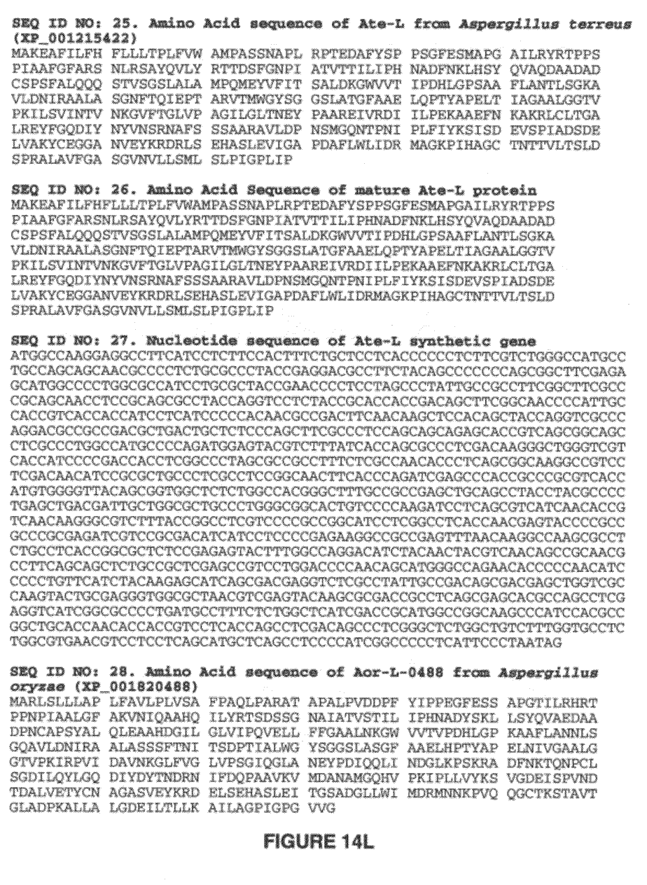

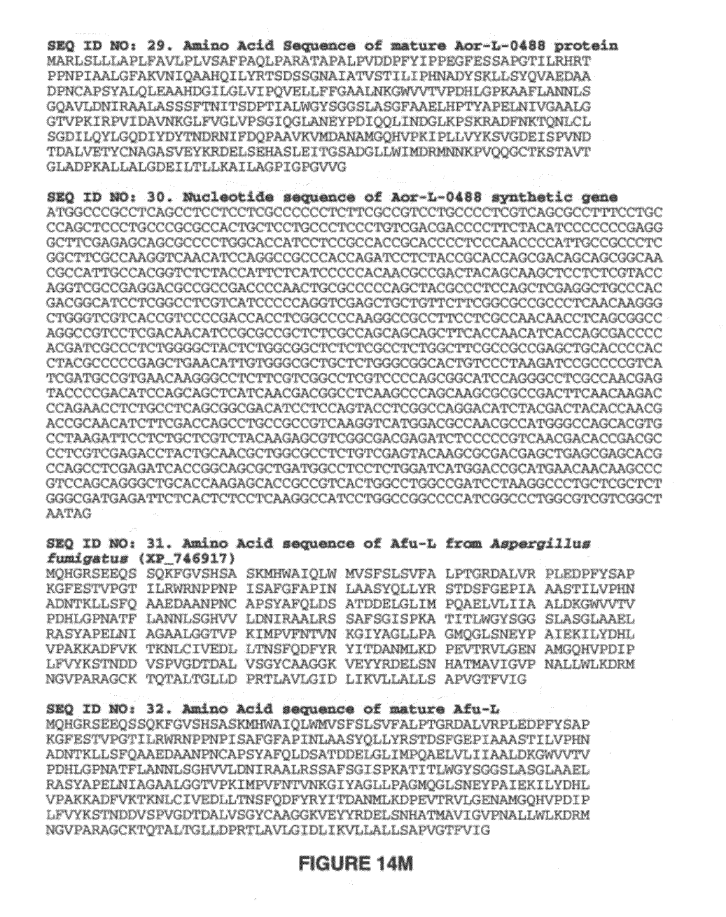

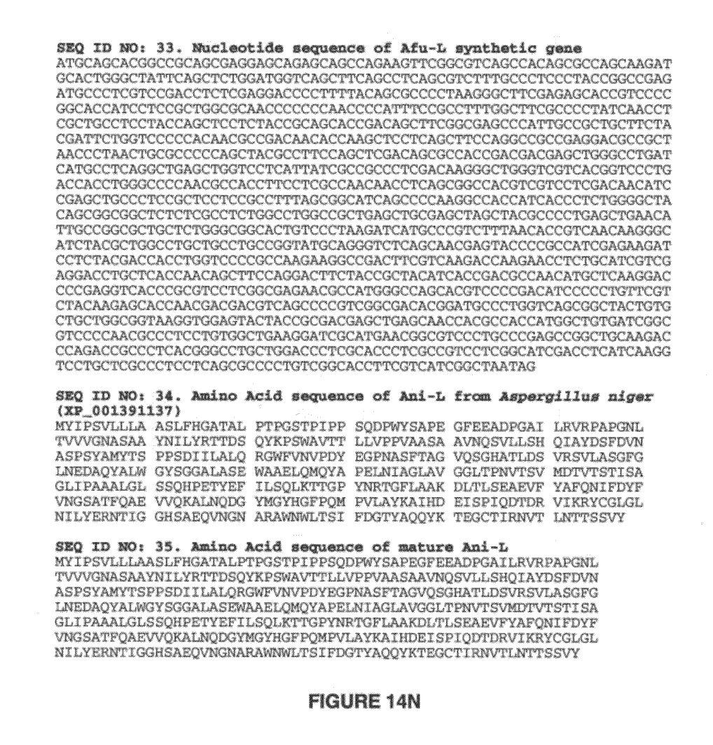

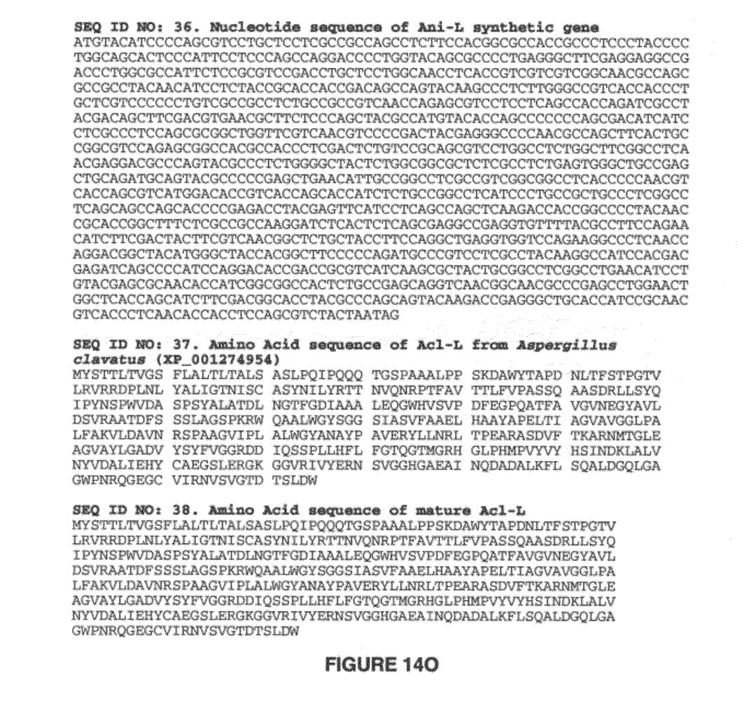

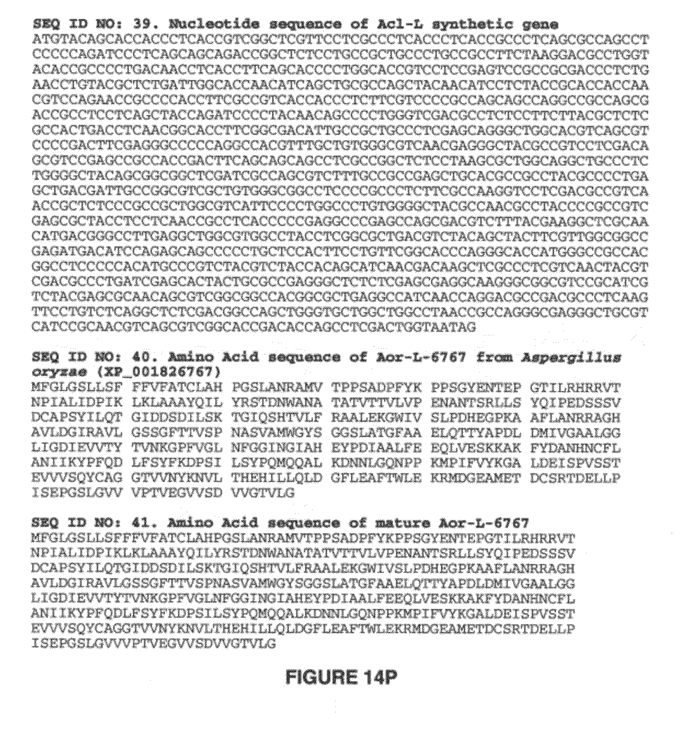

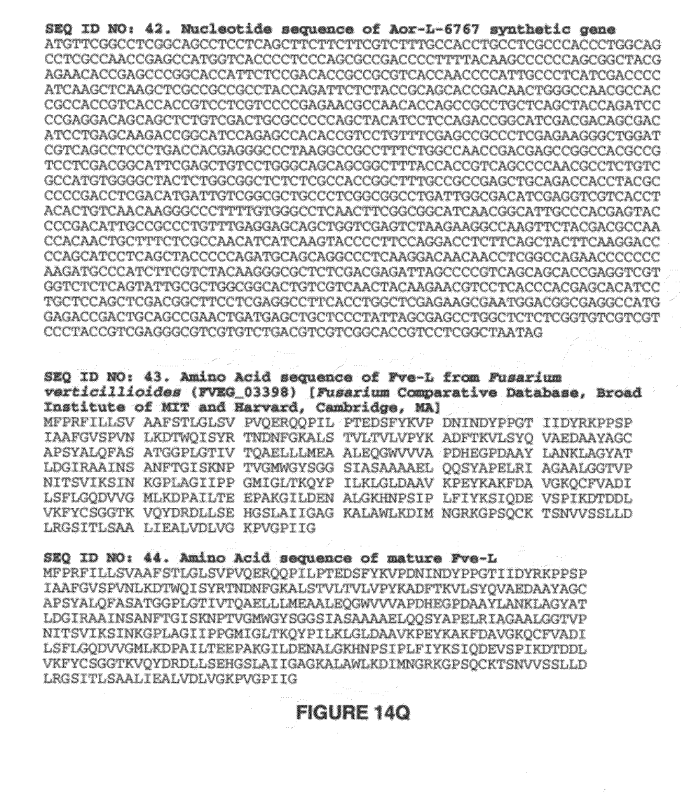

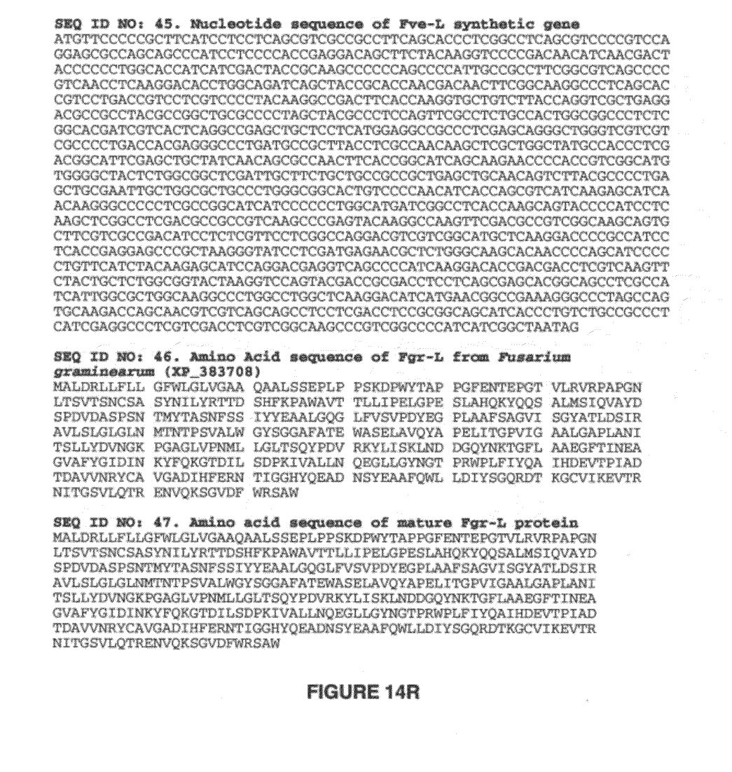

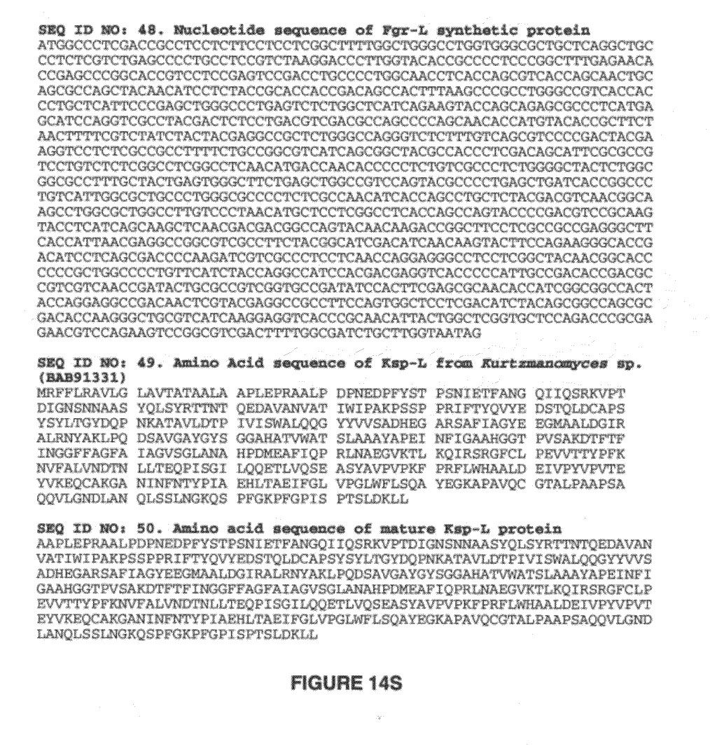

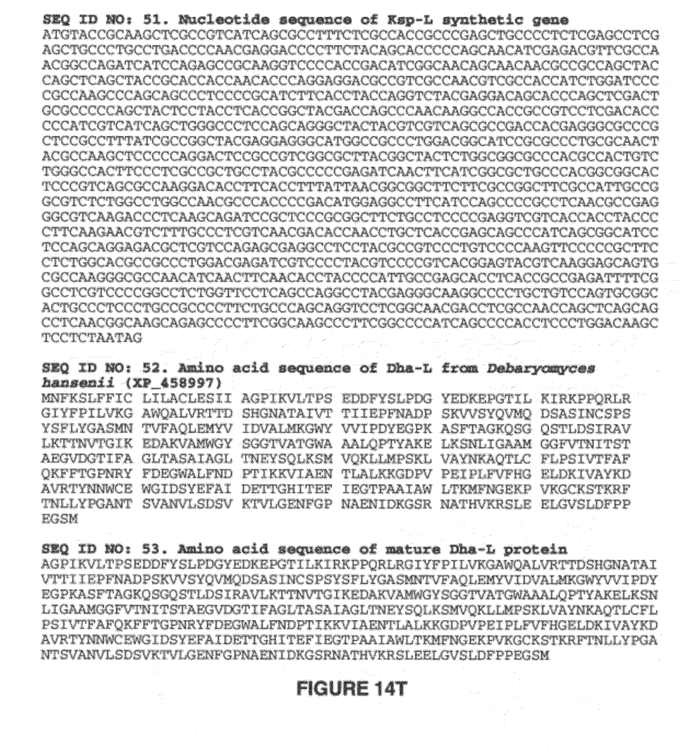

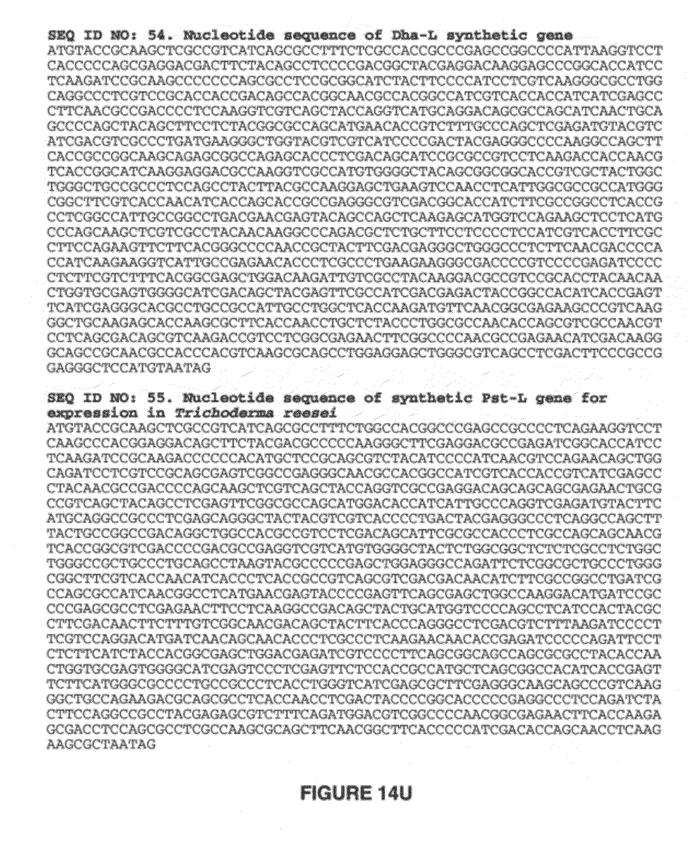

In some embodiments, CALA are expressed as secreted polypeptides, by relying on the naturally-occurring CALA signal sequence to mediate secretion, or by fusing the mature CALA polypeptide downstream of a heterologous signal sequence. The homologous signal sequences of each of exemplary CALA, i.e., Aad-L, Pst-L, Sco-L, Mfu-L, Rsp-L, Cje-L, Ate-L, Aor-L-0488, Afu-L, Ani-L, Acl-L, Aor-L-6767, Fve-L, Fgr-L, Ksp-L, and Dha-L, are evident by comparing their native “full-length” polypeptide sequence to their mature polypeptide sequences, which are listed in the Tables in FIGS. 14A-C. The complete amino acid and nucleotide sequences are shown in FIGS. 14D-U. Heterologous signal sequences include those from Cal-L and Cpa-L (described herein), from the Bacillus licheniformis amylase gene, and from the Trichoderma reesei cbh1 cellulase gene.

Expressing polypeptides in secreted form avoids the need to isolate the polypeptides from host cellular proteins, greatly reducing the amount of effort required to obtain relatively pure polypeptide product. In some cases, the cells media containing the secreted polypeptides can be used, at least in crude assays, directly and without purification. CALA can be further isolated from other cell and media components by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

In other embodiments, CALA are expressed as intracellular polypeptides, which do not require a signal sequence. Mature CALA polypeptides may be expressed in this manner, although addition purification steps are typically needed to sufficiently isolate the polypeptides from cellular proteins.

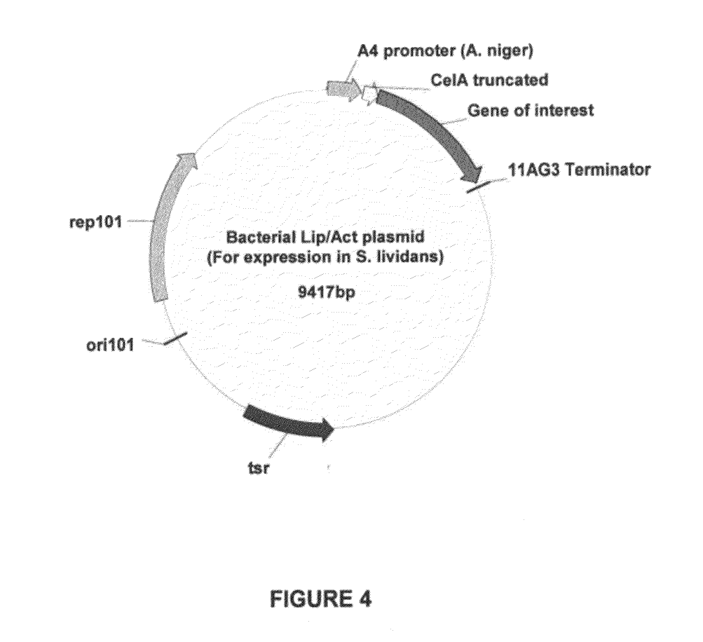

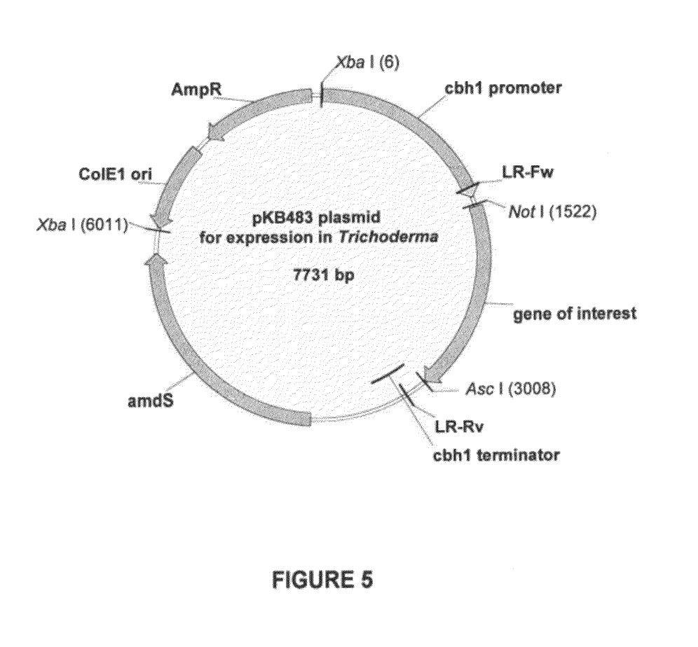

Exemplary methods for expressing each of the exemplary CALA are described here. For example, as described in Example 3, the Sco-L, Rsp-L, and Cje-L were expressed in the bacteria Streptomyces lividans, using a vector shown in FIG. 3. As described in Example 4, Aad-L and Pst-L, along with Cal-L and Cpa-L, were expressed in the methylotrophic yeast, Hansenula polymorpha, using a vector shown in FIG. 4. As described in Example 5, Mfu-I, Pst-L, Ate-L, Aor-L-0488, Afu-L, Ani-L, Acl-L, Aor-L-6767, Fve-L, Fgr-L, Ksp-L, and Dha-L were expressed in the filamentous fungus Trichoderma reesei, using a vector shown in FIG. 5.

Note that the Cal-L and Cpa-L were previously not expressed in Hansenula polymorpha; therefore, the present compositions and methods include expression of these lipases/acyltransferases in H. polymorpha.

### V. Cleaning Compositions and Methods Involving CalA-Related Polypeptides

Another aspect of the present compositions and methods is a detergent composition that includes one or more CALA, and a method of use, thereof.

The detergent compositions may be in dry or liquid form. Dry forms include non-dusting granules and microgranulates, as described in, e.g., U.S. Pat. Nos. 4,106,991 and 4,661,452. Dry formulations may optionally be coated with waxy materials, such as poly(ethylene oxide), (polyethyleneglycol, PEG), ethoxylated nonylphenols, ethoxylated fatty alcohols, fatty alcohols, fatty acids, and mono- and di- and triglycerides of fatty acids. Liquid forms include stabilized liquids. Such liquids may be stabilized by adding a polyol such as propylene glycol, a sugar or sugar alcohol, lactic acid or boric acid, or the like. Liquid forms may be aqueous, typically containing up to about 70% water, and up to about 30% organic solvent. Liquid forms can also be in the form of a compact gel type containing only about 30% water.

The detergent compositions will typically include one or more surfactants, each of which may be anionic, nonionic, cationic, or zwitterionic. The detergent will usually contain 0% to about 50% of anionic surfactant, such as linear alkylbenzenesulfonate (LAS); α-olefinsulfonate (AOS); alkyl sulfate (fatty alcohol sulfate) (AS); alcohol ethoxysulfate (AEOS or AES); secondary alkanesulfonates (SAS); α-sulfo fatty acid methyl esters; alkyl- or alkenylsuccinic acid; or soap. The composition may also contain 0% to about 40% of nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide.

The detergent compositions may optionally contain about 1% to about 65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst). The detergent may also be unbuilt, i.e. essentially free of detergent builder.

The detergent compositions may optionally contain one or more polymers. Examples include carboxymethylcellulose (CMC), poly(vinylpyrrolidone) (PVP), polyethyleneglycol (PEG), poly(vinyl alcohol) (PVA), polycarboxylates such as polyacrylates, maleic/acrylic acid copolymers and lauryl methacrylate/acrylic acid copolymers.

The detergent compositions may optionally contain a bleaching system, which may comprise a H₂O₂ source such as perborate or percarbonate, which may be combined with a peracid-forming bleach activator such as tetraacetylethylenediamine (TAED) or nonanoyloxybenzenesulfonate (NOBS). Alternatively, the bleaching system may comprise peroxy acids of e.g. the amide, imide, or sulfone type. The bleaching system can also be an enzymatic bleaching system, where a perhydrolase activates peroxide, as described in for example WO 2005/056783.

The detergent compositions may also contain other conventional detergent ingredients such as, e.g., fabric conditioners including clays, foam boosters, suds suppressors, anti-corrosion agents, soil-suspending agents, anti-soil redeposition agents, dyes, bactericides, optical brighteners, or perfume.

Detergents compositions may include one or more additional enzymes, such as an additional lipase, a cutinase, a protease, a cellulase, a peroxidase, a laccase, an aminopeptidase, amylase, carbohydrase, carboxypeptidase, catalase, cellulase, chitinase, cutinase, cyclodextrin glycosyltransferase, deoxyribonuclease, esterase, α-galactosidase, β-galactosidase, glucoamylase, α-glucosidase, β-glucosidase, haloperoxidase, invertase, laccase, lipase, mannosidase, oxidase, pectinolytic enzyme, peptidoglutaminase, peroxidase, phytase, polyphenoloxidase, perhydrolase, proteolytic enzyme, ribonuclease, transglutaminase, or xylanase, or the like.

The pH of a detergent (measured in aqueous solution at use concentration) is usually neutral or alkaline, e.g., pH about 7.0 to about 11.0, although the pH can be adjusted to suit a particular CALA. In general the properties of the selected one or more CALA should be compatible with the selected detergent composition and the CALA should be present in an effective amount.

Various types of detergent composition are contemplated, such as a hand or machine wash laundry detergent composition, including a laundry additive composition suitable for pre-treatment of stained fabrics and a rinse added fabric softener composition; a manual or machine dishwashing detergent composition; a detergent composition for use in general household hard surface cleaning operations, a composition for biofilm removal, and the like. Additional detergent composition include hand cleaners, shampoos, toothpastes, and the like.

In some embodiments, such compositions include a single CALA in a suitable amount, which can readily be determined using, e.g., the assays described herein. The amount may be from about 0.001% to about 1% of the total dry weight of the composition. Exemplary amounts are from about 0.001% to about 0.01%, from about 0.01% to about 0.1%, and from about 0.1% to about 1%. In some embodiments, such compositions include a plurality of CALA. In one particular embodiment, the composition includes a non-ionic ethoxylate surfactant and low water content, for example, DROPPS, and the CALA is Sco-L.

A related aspect of the present compositions and methods is the use of a detergent composition to remove oily soil or an oily stain from laundry, dishes, skin, or other surfaces using a detergent composition as described above. The method involved contacting the surface with a detergent composition that includes a CALA for period of time sufficient to hydrolyze the oily soil or stain, and then washing the detergent composition from the surface, e.g., with water, to leave behind a surface with reduced soil or stain.

### VI. Synthetic Reactions and Methods Involving CalA-Related Polypeptides

A. Formation of Peracetic Acid

Peracetic acid, also called peroxyacetic acid, is a strong oxidization agent that is effective for killing microorganisms and performing chemical bleaching of stains. Peracetic acid is mainly produced by combining acetic acid and hydrogen peroxide under aqueous conditions in the presence of sulfuric acid. Peracetic acid can also be produced by the oxidation of acetaldehyde, through the reaction of acetic anhydride, hydrogen peroxide, and sulfuric acid, and the reaction of tetraacetylethylenediamine in the presence of an alkaline hydrogen peroxide solution. An additional way to produce peracetic acid is enzymatically, by transferring an acyl group to a hydrogen peroxide donor using a suitable lipase/acyltransferase.

An aspect of the present compositions and methods is the formation of peracetic acid using one or more CALA. As described in detail in Example 11, several CALA were tested for their ability to form peracetic acid using a trioctanote donor and a hydrogen peroxide acceptor. Both Aad-L and the known lipase/acyltransferase, Cal-L, were effective in generating peracetic acid. It is expected that many of the present CALA will exhibit similar activity, since it is known that many lipases/acyltransferases are capable of forming peracetic acid.

In some embodiments, CALA are used to produce peracetic acid in situ, e.g., in a cleaning or bleaching composition, such that the peracetic acid is immediately available to react with a target organism or stain (see, e.g., WO2005/056782).

B. Manufacture of Perfumes and Fragrances

One of the most common industrial applications involving acyltransferase reactions is in the manufacture of ester compounds for use in perfumes and fragrances. These reactions typically occur in an aqueous environment and the donor and acceptor molecules are selected to impart desired fragrance characteristics on the final ester product.

An aspect of the present compositions and methods is the use of one or more CALA to produce fragrant esters for use in perfumes and fragrances via an acyltransferase or transesterification reaction. As described in Example 8, including Table 4, the present CALA were able to use donor molecules having a variety of different chain-lengths, ranging from 4 to 18 carbons. Different CALA had different chain-length preferences. For example, Cpa-L and Mfu-L had a preference for C8 donors, Pst-L, Cal-L, and Aad-L had a preference for C10 donors, and Sco-L had a preference for C16 donors. LIPOMAX™ (i.e., Pseudomonas alcaligenes variant M21L lipase) had a preference for C10 donors. While only certain donors were tested, CALA are expected to demonstrate similar results using other donors and any number of acceptors, making them useful for performing acyltransferase/transesterification reactions involving a variety of donor and acceptor molecules. The use of lipases/acyltransferase in the manufacture of perfumes and fragrances is discussed in, e.g., Neugnot V. et al. (2002) Eur. J. Biochem. 269:1734-45; Roustan, J. L. et al. (2005) Appl. Microbiol. Biotechnol. 68:203-12; and WO 08106215).

C. Formation of Surfactants

Another common industrial application for lipases/acyltransferase is in the production of fatty acid esters surfactants. Such surfactants may function as emulsifiers in food products. The surfactants may be produced in a reaction and then added to a food product, or may be generated in situ in the process of preparing the food product, i.e., by including a lipase/acyltransferas in the raw or partially processed ingredients of the food product.

An aspect of the present compositions and methods is the use of one or more CALA to produce surfactants that function as emulsifiers in food products. As described in Example 13, including Table 8, several CALA were able to generate propylene glycol esters of fatty acids using triolein as a substrate and 1,2-propanediol and 1,3-propanediol as an acceptors. CALA capable of producing esters of fatty acids included Aad-L, Pst-L, Sco-L, Mfu-L, Cje-L, and the known CALA Cal-L and Cpa-L. While only certain CALA, donors, and acceptors were tested, others are expected to demonstrate similar results. Krog, N. (2008) Food Emulsifiers—Chemical Structure and Physico-chemical Properties, Technical Paper 18-1e, Danisco A/S, Denmark; Friberg, S. et al. (2003) Food Emulsions, Edition 4, CRC Press, 640 pp.; Karsa, D. R. (1999) Design and selection of performance surfactants, CRC Press, 364 pp.

D. Degumming of Vegetable Oil

Crude vegetable oil contains phospholipids, which possess a phosphate ester in place of a fatty acid side chain. The major phospholipids found in soybean, canola, and sunflower oils are phosphatidylcholine (PC) and phosphatidylethanolamine (PE), with lesser amounts of phosphatidylinositol (PI) and phosphatidic acid (PA) being present. Phospholipids impart undesirable flavors to vegetable oil, affect its stability and appearance, and interfere with chemical reactions.

The removal of phospholipids may accomplished by a refining step known as degumming, which relies on the amphiphilic nature of phospholipids compared to other triglycerides. Briefly, the addition of water to vegetable oil causes the hydration of phospholipids, which form a gum than can be separated by centrifugation. However, because phospholipids are effective emulsifiers, they trap triglycerides, resulting in the loss of desirable lipid components during degumming. To avoid trapping triglycerides, phospholipid-specific lipases can be used to hydrolyze the phospholipids and alter their emulsification properties. The resulting phospholipids can still be removed by degumming but with reduced loss of triglycerides.

An aspect of the present compositions and methods is the use of one or more CALA to hydrolize phospholipids present in vegetable oil, to reduce the loss of triglycerides during a degumming process.

E. Manufacture of Biofuels or Synthetic Oils

The synthesis of fatty acid esters from vegetable oils is central to the production of biofuels, such as biodiesel, and synthetic oils, such as those based on, e.g., diesters, polyolesters, alklylated napthlenes, alkyklated benzenes, polyglycols, and the like. The chemistry for making biofuels and synthetic oils is generally straightforward, with the main limitations being the cost of starting materials and reagents for synthesis. In addition, enzymatic transesterification for the production of biodiesel has been suggested for producing a high purity product in an economical, environment friendly process, under mild reaction conditions.

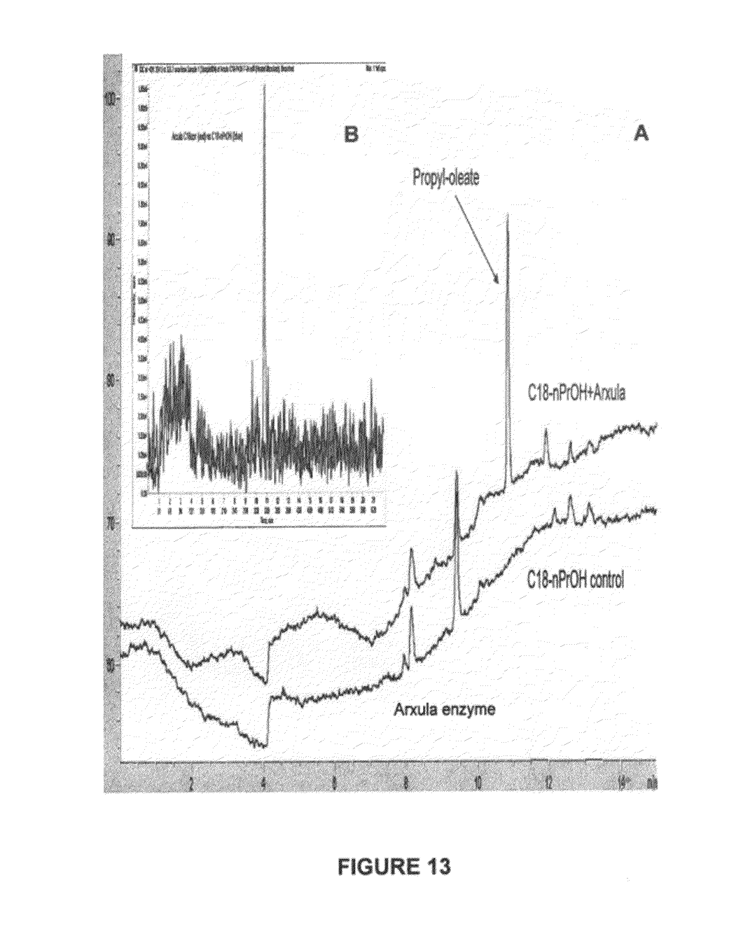

An aspect of the present compositions and methods is the use of one or more CALA to produce biofuels or synthetic oils via an acyltransferase or transesterification reaction. As described in Example 14, and shown in FIG. 13, two CALA (Aad-L and Pst-L) were tested for their ability synthesize methyl and ethyl esters using triolein as a donor and methanol or ethanol and acceptors. Other CALA are expected to exhibit similar activity, and other donor and acceptor molecules are expected to be suitable starting materials for synthesis. As described above and in Example 8, several CALA were characterized to determine their donor specificity, which information can be used to select suitable starting material for synthesis. Biofuel synthesis is described in, e.g., Vaysse, L. et al. (2002) Enzyme and Microbial Technology 31:648-655; Fjerbaek, L. et al. (2009) Biotechnol. Bioeng. 102:1298-315; Jegannathan, K. R. et al. (2008) Crit. Rev. Biotechnol. 28:253-64.

F. Other Synthetic Reactions

In addition to the above-described synthetic reactions, CALA can be used in molecular biology applications to acylate proteins and nucleic acids, to acylate molecules in making pharmaceutical compounds, and in other reactions where the transfer of an acyl group is desirable. Further aspects of the present compositions and methods relate to the use of CALA in such reactions.

Other features of the compositions and methods will be apparent in view of the description.

### EXAMPLES

The following examples are intended to illustrate, but not limit, the invention.

### Example 1

### Assay Procedures

Various assays were used in the following Examples, which are set forth below for ease in reading. Any deviations from the protocols provided, below, are specified in subsequent Examples.

### 1. Hydrolysis of Synthetic Ester Substrates to Determine Lipase/Esterase Activity

A. Para-nitrophenyl butyrate (pNB) Assay to Determine Lipase/Esterase Activity

### Equipment:

Specrophotometer capable of kinetic measurements and temperature control

Water bath at 25° C.

96-well microtiter plates

### Materials:

Assay buffer: 50 mM HEPES pH 8.2, 6 gpg, 3:1 Ca:Mg Hardness, 2% poly(vinyl) alcohol (PVA; Sigma 341584)

Substrate: 20 mM p-Nitrophenyl Butyrate (pNB; Sigma, CAS 2635-84-9, catalog number N9876) dissolved in DMSO (Pierce, 20688, Water content <0.2%), stored at −80° C. for long term storage

### Procedure:

Serial dilutions of enzyme samples in assay buffer were prepared in 96-well microtiter plates and equilibrated at 25° C. 100 μL of 1:20 diluted substrate (in assay buffer) was added to another microtiter plate. The plate was equilibrated to 25° C. for 10 minutes with shaking at 300 rpm. 10 μL of enzyme solution from the dilution plate was added to the substrate containing plate to initiate the reaction. The plate was immediately transferred to a plate-reading spectrophotometer set at 25° C. The absorbance change in kinetic mode was read for 5 minutes at 410 nm. The background rate (with no enzyme) was subtracted from the rate of the test samples.

B. Para-nitrophenyl palmitate (pNPP) Assay to Determine Lipase/Esterase Activity

The pNPP assay to measure lipase/esterase activity was performed exactly as described in the pNB assay except that the substrate used was 20 mM p-Nitrophenyl Palmitate (pNPP; Sigma, CAS 1492-30-4, catalog number N2752) dissolved in DMSO (Pierce, 20688, Water content <0.2%), stored at −80° C. for long term storage and Triton-X 100 was added at 2% in the reaction.

C. Chain Length Dependence Assay to Determine Carbon Chain Length Preference

To measure lipase/esterase activity as a function of carbon chain length, all substrates (pNB: para-Nitrophenyl butyrate: C4:0 (Sigma, CAS 2635-84-9, catalog number N9876); pNO: para-Nitrophenyl octanoate: C8:0 (Alfa Aesar (Ward Hill, Mass.), Catalog #L12022), pND: para-Nitrophenyl decanoate: C10:0 (Fluka, Catalog #21497, CAS 1956-09-8); pNP: para-Nitrophenyl palmitate: C16:0 (Sigma, CAS 1492-30-4, catalog number N2752), and pNS: para-Nitrophenyl stearate: C18:0 (Sigma, Catalog #N3502, CAS 104809-27-0) were suspended in isopropanol to a concentration of 20 mM. Substrates were diluted to 1 mM in assay buffer (50 mM HEPES, 2% PVA, 2% Triton X-100, 6 gpg). To measure activity, 100 μL of each chain length substrate in assay buffer was added to a 96-well microtiter plate. 10 μL of appropriately diluted enzyme aliquots was added to the substrate containing plate to initiate the reaction. The plate was immediately transferred to a plate reading spectrophotometer set at 25° C. The absorbance change in kinetic mode was read for 5 minutes at 410 nm. The background rate (with no enzyme) was subtracted from the rate of the test samples.

### 2. Triglyceride and Ester Hydrolysis Assay in 96-Well Microtiter Plates

This assay was designed to measure enzymatic release of fatty acids from triglyceride or ester substrate. The assay consists of a hydrolysis reaction where incubation of enzyme with a an emulsified substrate results in liberation of fatty acids, detection of the liberated fatty acids and measurement in the reduction of turbidity of the emulsified substrate.

### Equipment:

Plate Reading Spectrophotometer capable of end point measurements (SpectraMax Plus384 (Molecular Devices, Sunnyvale, Calif.)

96-well microtiter plates

Eppendorf Thermomixer

### Substrates:

Glycerol trioctanoate (Sigma, CAS 538-23-8, catalog number T9126-100mL),

Glyceryl trioleate (Fluka, CAS 122-32-7, catalog number 92859)

Glyceryl tripalmitate (Fluka, CAS 555-44-2, catalog number 92902)

Cholesteryl linoleate (Sigma, catalog number C0289-1G)

Phophatidylcholine (Sigma, catalog number P3644-25G)

Tween-80 (Sigma, catalog number P1754-500 ml)

Ethyl oleate (Sigma, catalog number 268011-5G)

Ethyl palmitate (Sigma, catalog number P9009-5G)

### Reagents:

NEFA (non-esterified fatty acid) assay reagent (HR Series NEFA-HR (2) NEFA kit, WAKO Diagnostics, Richmond, Va.)

### Procedure:

Emulsified triglycerides (0.75% (v/v or w/v)) were prepared by mixing 50 ml of gum arabic (Sigma, CAS 9000-01-5, catalog number G9752; 10 mg/ml gum arabic solution made in 50 mM MOPS pH 8.2), 6 gpg water hardness, in 50 mM HEPES, pH 8.2) with 375 μL of triglyceride (if liquid) or 0.375 g triglyceride (if solid). The solutions were mixed and sonicated for at least 2 minutes to prepare a stable emulsion.

200 μL of emulsified substrate was added to a 96-well microtiter plate. 20 μL of serially-diluted enzyme samples were added to the substrate containing plate. The plate was covered with a plate sealer and incubated at 40° C. shaking for 1-2 hours. After incubation, the presence of fatty acids in solution was detected using the HR Series NEFA-HR (2) NEFA kit as indicated by the manufacturer. The NEFA kit measures non-esterified fatty acids.

### 3. Triglyceride Hydrolysis Assay on Microswatches to Determine Lipase Activity

Microswatches treated with triglycerides were prepared as follows. EMPA 221 unsoiled cotton fabrics (Test Fabrics Inc. West Pittiston, Pa.) were cut to fit 96-well microtiter plates. 0.5-1 μL of neat trioctanoate was spotted on the microswatches. The swatches were left at room temperature for about 10 minutes. One triglyceride treated microswatch was placed in each well of a microtiter plate. DROPPS™ detergent (0.1%) (Laundry propps, Cot'n Wash Inc., Ardmore, Pa.) or 50 mM HEPES pH 8.2, 6 gpg, 2% PVA (polyvinyl alcohol) was added to each well containing a microswatch. DROPPS is a detergent composition having only a non-ionic ethoxylate surfactant and very low water content (about 10% by weight). 10 μL of serially diluted enzyme samples were added to these wells. The plate was sealed with a plate sealer and incubated at 750 rpm at 40° C. for 60 minutes. After incubation, the supernatant was removed (and saved) from the swatches and the swatches were rinsed with 100 uL of detergent (save rinse) and blotted dry on paper towels. The presence of fatty acids in solution (supernatant and rinse) and remaining on the cloth was detected using the HR Series NEFA-HR (2) NEFA kit (WAKO Diagnostics, Richmond, Va.) as indicated by the manufacturer.

### 4. Assay to Measure Peracetic Acid Formation by CALA

Stock solutions: 125 mM citric acid (Sigma P/N C1857), pH to 5.0 with NaOH, 100 mM ABTS (2,2′-Azino-bis(3-ethylbenzothiazoline-6-sulfonic acid) Diammonium salt, Fluka P/N WA10917 prepared in distilled H₂O, 25 mM KI (Sigma P4286 prepared in distilled H₂O. The working substrate solution consisted of 50 mL of 125 mM citric acid buffer+500 μL of ABTS stock+100 μL of 25 mM KI stored in a light proof container.

Procedure: A standard curve for peracetic acid was prepared by making serial dilutions (1:100 dilution in 125 mM citric acid) of stock peracetic acid (Sigma-Fluka P/N 77240). 20 μL of all standard solutions and test samples were added to wells in a 96-well microtiter plate in triplicates. 200 μL of working substrate solution was added to each well of the microtiter plate. The reaction was allowed to proceed for 3 minutes at room temperature and the change in absorbance at 420 nm was monitored in a standard UV-Vis spectrophotometer.

### 5. Spot Assay for Detection of CALA Lipase Activity in Culture Supernatants

Cells from cultures of Trichoderma, Hansenula and Streptomyces were separated from supernatants by centrifugation and the supernatants were analyzed for lipase activity using the agar spot assay. Screening for lipase/acyltransferase producers on agar plates is based on the release of fatty acid from the substrates (tributyrin, olive oil, bacon fat, egg yolk, or phosphatidylcholine) in the presence of lipase.

The assay plate contained 2.0 g Bacto Agar (dissolved in 100 ml 50 mM sodium phosphate buffer pH 5.5 by heating for 5 minutes). The solution was kept at 70° C. in a water bath and while stiffing, 0.5 ml 2% Rhodamine and 40 ml tributyrin, olive oil, bacon fat, or egg yolk were added to it. The mixture was subjected to sonication for 2 minutes and 10-15 ml was poured into petri dishes. Following cooling of the plates, holes were punched into the agar and 10 μL of culture supernatants were added into the holes. The plates were incubated at 37° C. until pink color was detected indicating the presence of lipolytic activity. The pink color is formed when fatty acids released from hydrolysis of substrates by lipase form a complex with Rhodamine B.

### 6. Enzyme Sample Preparation

Enzymes used in biochemical studies were ultra-filtered concentrates or media supernatant from cell growth. Protein concentration was estimated using densitometry. Bovine serum albumin was used to construct a standard curve from which the concentration of protein samples was then determined. In some cases, enzyme concentration was not calculated and activity was measured in relation to a reference enzyme.

### Example 2

### Identification of Genes with Sequence Identity/Similarity to Known Lipases/Acyltransferases from Candida spp.

Experiments were conducted to identify genes encoding enzymes with lipase and/or acyltransferase activities in published sequence databases. The amino acid sequences of two functionally-characterized lipases/acyltransferases, namely the extracellular lipases/acyltransferases, Cpa-L from Candida parasilopsis (U.S. Pat. No. 7,247,463) and Cal-L from Candida albicans described by Roustan et al. (2005) Applied Microbiology & Biotechnology 68:203-212, were used as queries in BLAST analyses on the non-redundant (nr) protein database of the National Center for Biotechnology Information (NCBI). In addition, the multi-fungi blast query was used to search for acyltransferases in all fungal genomic sequences for the organisms hosted at the Broad Institute, which is available on-line.

Using these two sequence databases, lipases/acyltransferases were identified in different ascomycetes, in particular the filamentous fungi Aspergillus sp. and Fusarium sp., in different yeasts such as Candida sp., Debaryomyces hansenii, Arxula adeninovirans (Aad-L), Pichia stiptis (Pst-L), Kurtzmanomyces sp. and Malassezia sp. (Malassezia furfur) (Mfu-L). Lipase/acyltransferases were also found in prokaryotic organisms in both gram positive and gram negative bacteria namely, Streptomyces sp. (Streptomyces coelicolor) (Sco-L), Mycobacterium sp., Rhodococcus sp. (Rsp-L), and Corynebacterium sp. (Corynebacterium jeikeium) (Cje-L). These amino acid sequence were previously annotated as secretory lipases or unknown hypothetical proteins, and may represent members of a new family of lipases/acyltransferases found in both eukaryotic and prokaryotic organisms, collectively referred to herein as CalA-related lipases/acyltransferases (CALA).

FIGS. 1A-J show a partial amino acid sequence alignment of the different CALA using the multiple sequence alignment application, AlignX, which is part of the Vector NTI Advance application (Invitrogen, Carlsbad, Calif., USA), a program based on the Clustal W algorithm that is designed to perform and manage multiple sequence alignment projects. Align X incorporates all of the following features: profile alignment, guide tree construction, display in graphical representation, use of residue substitution matrices, and secondary structure consideration. A guide tree, which resembles a phylogenetic tree, is built using the neighbor joining (NJ) method of Saitou and Nei (Saitou, N. and Nei, M. (1987) Mol. Biol. Evol. 4:406-25). The NJ method works on a matrix of distances between all pairs of sequence to be analyzed. These distances are related to the degree of divergence between the sequences. The guide tree is calculated after the sequences are aligned. AlignX displays the calculated distance values in parenthesis following the molecule name displayed on the tree.

The alignment shows a first conserved amino acid motif, having the consensus sequence GYSGG, and which is present in CALA from both eukaryotic and prokaryotic organisms. A second conserved motif, having the consensus sequence YAPEL, is also present in CALA from both eukaryotic and prokaryotic organisms. These conserved sequences are indicated with bold text.

Table 1 shows the relative amino acid sequence homology among the different CALA. All have below 49% homology to the known CALA Cpa-L (U.S. Pat. No. 7,247,463). In particular, all the heretofore uncharacterized CALA have between about 18% and 49% homology to Cal-L.

TABLE 1
Homology among CALA lipases/acyltransferases

| | | | % Sequence identity to: | |
|---|---|---|---|---|
| | | | Candida | Candida |
| | | | albicans | parasilopsis |
| | | | (Cal-L) | (Cpa-L) |
| Organism | Enzyme | Accession No. | XP_712265 | CAC86400 |
| Arxula adeninivorans | Aad-L | CAI51321 | 40.56 | 37.22 |
| Pichia stipitis | Pst-L | XP_001386828 | 49.02 | 46.77 |
| Streptomyces coelicolor | Sco-L | NP_631446/CAB76297 | 22.89 | 22.42 |
| Malassezia furfur | Mfu-L | AAZ85120 | 29.12 | 29.46 |
| Rhodococcus sp. | Rsp-L | YP_701197 | 31.93 | 28.46 |
| Corynebacterium jeikeium | Cje-L | YP_250713/NC_007164/ | 19.95 | 19.50 |
| | | CAI37095 | | |
| Aspergillus terreus | Ate-L | XP_001215422 | 34.96 | 34.18 |
| Aspergillus oryzae | Aor-L- | XP_001820488 | 37.88 | 34.33 |
| | 0488 | | | |
| Aspergillus fumigatus | Afu-L | XP_746917 | 34.65 | 34.33 |
| Aspergillus niger | Ani-L | XP_001391137 | 31.75 | 32.18 |
| Aspergillus clavatus | Acl-L | XP_001274954 | 27.50 | 28.92 |
| Aspergillus oryzae | Aor-L- | XP_001826767 | 34.27 | 34.65 |
| | 6767 | | | |
| Fusarium verticillioides | Fve-L | FVEG_03398 | 35.65 | 33.95 |
| Fusarium graminearum | Fgr-L | XP_383708 | 30.32 | 30.03 |
| Kurtzmanomyces sp. | Ksp-L | BAB91331 | 30.81 | 29.32 |
| Debaryomyces hansenii | Dha-L | XP_458997 | 44.23 | 44.40 |

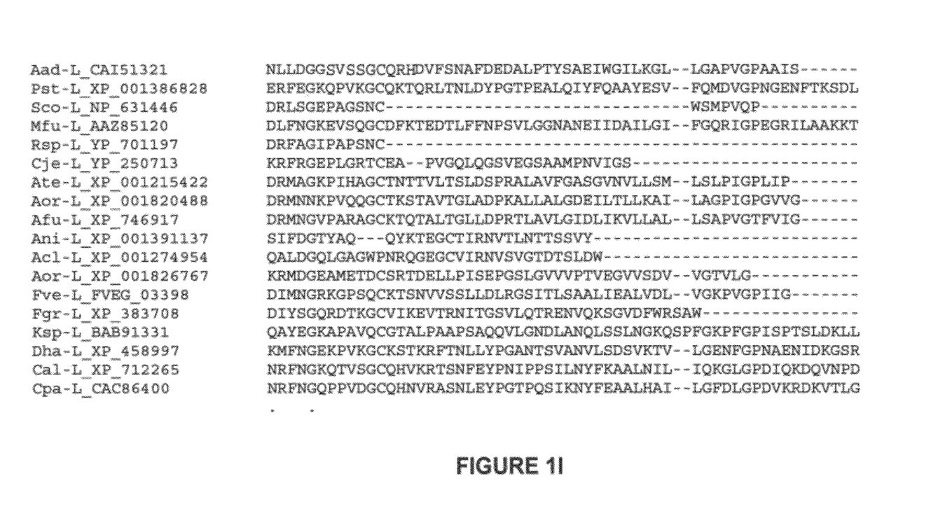

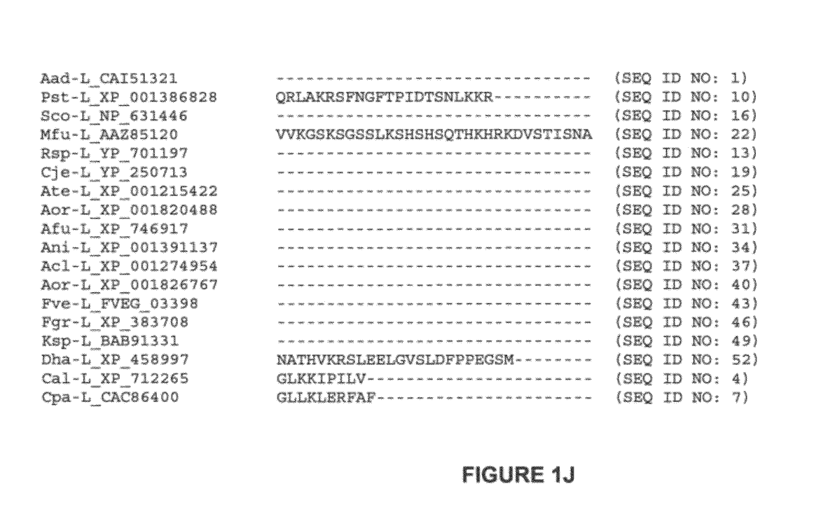

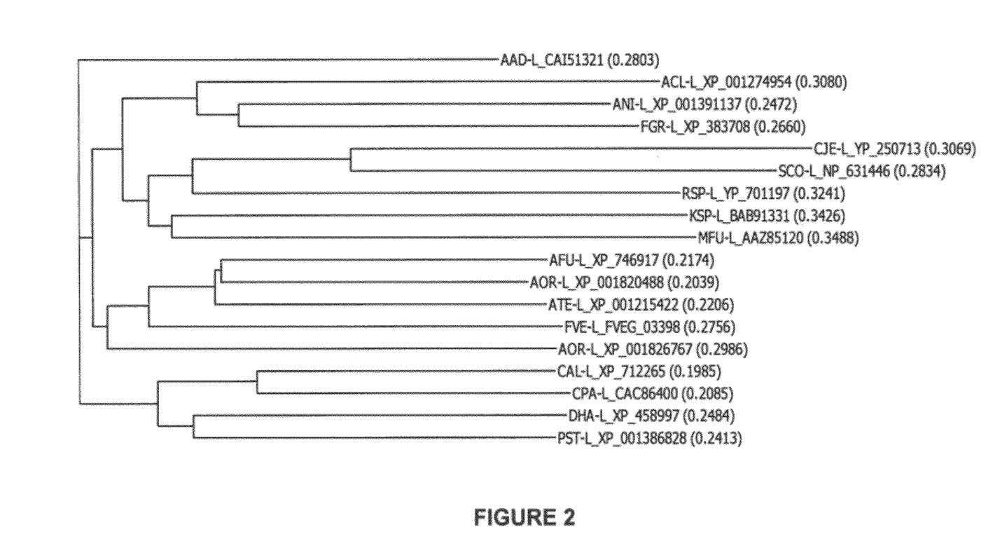

FIG. 2 shows a dendrogram based on the protein sequence similarity to Cpa-L and other known and putative lipase/acyltransferases. The dendrogram shows clustering of CALA identified in bacteria. Yeast CALA are clustered together with the known CALA Cpa-L and Cal-L, while the sequences from filamentous fungi aggregated in two separate clusters.

A common feature of the CALA is that they have molecular weights higher than those of the typical fungal lipases. In particular, CALA are at least 390 amino acid residues in length (including the signal peptide), and in many cases greater than 400 amino acids in length. CALA have a deduced molecular weight of at least 39 kDa. Glycosylation sites are present in the CALA from eukaryotic organism, which may additionally increase mass when expressed in fungal hosts. In contract, most lipases described in the literature and in the patent databases have shorter polypeptide chains and molecular weights of less than 39 kDa. In particular, the lipase 3 gene from Aspergillus tubigenesis, described in U.S. Pat. No. 6,852,346, encodes a protein with 297 amino acid residues and a molecular weight of around 30 kDa. The detergent enzyme, LIPEX™ from Humicola lanuginosus has 269 amino acids (mature protein).

### Example 3

### Cloning and Expression of CALA from Arxula, Pichia and Candida in Hansenula polymorpha

The plasmid pFPMT121 (a derivative of plasmid pFMD-22a described in Gellissen et al. (1991) Bio/Technology 9:291-95) was used as the expression vector for the expression of several CALA in the methylotrophic yeast, Hansenula polymorpha. These CALA included Aad-L from Arxula adeninivorans, Pst-L from Pichia stipitis and the two known lipases/acyltransferases from Candida albicans (Cal-L) and Candida parasilopsis (Cpa-L). The two Candida lipases/acyltransferases were used as controls. Synthetic genes encoding Aad-L, Pst-L, Cal-L, and Cpa-L were designed based on the published amino acid sequences, (i.e., CAI51321, XP_{—}001386828, Cal-L-XP_{—}712265, and Cpa-L-CAC86400, respectively), using codon selection methods for improving expression in H. polymorpha. The synthetic genes were inserted into the EcoRI-BamHI sites of the pFPMT121 polylinker to generate plasmids pSMM (FIG. 3). The Arxula signal sequence in Aad-L was replaced by the Saccharomyces cerevisea alpha factor pre-pro-signal sequence (Waters, G. et al. (1988) J. Biol. Chem. 263:6209-14), which was fused upstream of the mature protein sequence. Pst-L from Pichia stipitis was expressed using its own signal peptide. The Saccharomyces alpha factor pre-pro signal peptide was fused to the mature proteins of both known lipases/acyltransferase from Candida. The yeast strain H. polymorpha RB11, which is deficient in oritidine 5′-phosphate decarboxylase (ura3) (Roggenkamp et al. (1986) Molecular and General Genetics 202:302-08; Rhein Biotech, Düsseldorf), served as host for transformation. H. polymorpha strain was transformed by electroporation of competent cells as described by Faber et al. (1994) Curr. Genet. 25:305-10).

Competent cells for transformation were prepared as follows: 5 ml overnight culture was grown on non-selective YPD medium (1% yeast extract, 2% peptone, and 2% glucose) at 37° C. The culture was diluted 50-fold in 200 ml pre-warmed YPD medium and grown at 37° C. to an OD600=1.0. Cells were harvested by centrifugation at 3,000×g for 5 minutes at room temperature and resuspended in 20 ml pre-warmed (37° C.) PPD buffer, containing 50 mM potassium phosphate buffer pH 7.5 and 25 mM DTT. The cells were incubated on ice for 15 minutes at 37° C. and then harvested by centrifugation at 3,000×g for 5 minutes at room temperature. After the last wash and centrifugation, the cells were kept on ice and resuspended in 1 ml STM buffer (270 mM sucrose, 10 mM Tris-HCl pH 7.5, and 1 mM MgCl₂). For transformation, 60 μL of competent cells were mixed with 1 μL of pSMM plasmid DNA and transferred into the electroporation cuvette (E-shot, 0.1 cm standard electroporation cuvette from Invitrogen, Carlsbad, Calif., USA). Electroporation settings were 16 kV/cm, 25 μF, and 50Ω. After electroporation, 1 ml of YPD medium (room temperature) was added to the cell/DNA mixture. The cell suspension was then incubated for 1 h at 37° C. without agitation. Cells were harvested (5 min, 3,000×g), washed once, and subsequently resuspended (and diluted) in YNB medium (0.14% Difco yeast nitrogen base w/o amino acids supplemented with 1% glucose), spread on YNB selective plates, and incubated at 37° C.

Multicopy strains of H. poymorpha were obtained by sequential culturing of uracil prototrophic transformants in selective minimal medium and rich medium for several cycles of growth as described by Gelisen et al. (1991) Biotechnology 9:291-95. Single transformants were grown in bulk (i.e., 50 colonies per single shake flask). Briefly, 50 colonies were picked and incubated in 200 ml shake flasks containing 20 ml YNB medium at 37° C. with shaking at 200 rpm for 2 days. After incubation, 100 μL of these cultures were used to inoculate fresh medium and the process was repeated seven times (for a total of eight passages). For stabilisation of transformants, 20 ml YPD medium in 200 ml flasks were inoculated with 50 μL of the final passage cultures and incubated with shaking at 200 rpm at 37° C. This step was repeated twice.

Finally, the cultures were plated on YNB-glucose plates and incubated at 37° C. for 4 days to obtain mitotically stable transformants. Single colonies of stable transformants derived from the YNB-plates were inoculated in 3 ml of YPD medium overnight at 37° C. The next day, 500 μL of cultures were used to inoculate 15 ml of YNB-medium containing 1% glycerol and incubated for 3-4 days at 28° C. Supernatants from these cultures were used to assay for lipase activity using the spot assay. For protein production, Hansenula transformants expressing the CALA of interest were cultivated in fermenter tanks as described in U.S. Pat. No. 7,455,990. Aliquots of ultrafiltered concentrate (UFC) from the tanks were used for biochemical assays.

### Example 4

### Cloning and Expression of CALA from Streptomyces coelicolor, Rhodococcus sp. (RHA1), and Corynebacterium jeikeium K411 in Streptomyces lividans

Synthetic genes for the Streptomyces coelicolor, Rhodococcus sp. (RHA1), and Corynebacterium jeikeium K411 lipases were ordered from Geneart AG (Regensburg, Germany) and GeneRay Biotech (Shanghai, China) for extracellular expression in Streptomyces lividans. The CelA signal sequence (obtained from the pKB105 plasmid, described in U.S. Publication No. 2006/0154843) was fused in front of the Rsp-L and Cje-L mature proteins. Sco-L was cloned and expressed using its own signal sequence. The synthetic genes were inserted into the NcoI/BamH1 sites of the expression vector, pKB105 to generate bacterial Lip/Act plasmids separately containing each of the Sco-L, Rsp-L and Cje-L CALA (FIG. 4).

The host Streptomyces lividans TK23 derivative strain was transformed with the bacterial Lip/Act plasmids according to the protoplast method described in Kieser et al. (2000) Practical Streptomyces Genetics, The John Innes Foundation, Norwich, UK. Transformed cells were plated on R5 selection plates and incubated at 30° C. for 3 days. Several transformants from the Streptomyces transformation plate was inoculated in TSG medium (see, below) in shake flasks at 28° C. for 3 days. Cultures were then transferred to a Streptomyces 2 Modified Medium (see, below) and incubated for an additional 4 days at 28° C. Supernatants from these cultures were used to assay for lipase activity using the spot assay. Media/reagents are described, below.

### TSG Medium:

16 g BD Difco tryptone, 4 g BD Bacto soytone, 20 g Sigma caseine (hydrolysate), and 10 g potassium phosphate, dibasic, brought to 1 liter. After autoclaving, 50% glucose was added to a final concentration of 1.5%.

### Streptomyces Production 2 Modified Medium:

2.4 g citric acid monohydrate, 6 g Biospringer yeast extract, 2.4 g ammonium sulfate, 2.4 g magnesium sulfate heptahydrate, 0.5 ml Mazu DF204 (antifoam), 5 ml Streptomyces modified trace elements (1 liter stock solution contains: 250 g citric acid monohydrate, 3.25 g FeSO₄.7H₂O; 5 g ZnSO₄.7H₂O, 5 g MnSO₄.H₂O, 0.25 g H₃BO₃). The pH was adjusted to 6.9. After autoclaving, 2 ml 100 mg/ml calcium chloride, 200 ml 13% (w/v) potassium phosphate, monobasic (pH 6.9), and 20 ml 50% glucose were added.

### R5 Plates:

206 g sucrose, 0.5 g K₂SO₄, 20.24 g MgCl₂, 20 g glucose, 0.2 g Difco casamino acids, 10 g Difco yeast extracts, 11.46 g TES, 4 g L-Asp, 4 ml of trace elements, 44 g Difco agar, 20 ml 5% K₂HPO₄, 8 ml 5M CaCl₂.2H₂O and 14 ml 1N NaOH were added to a final volume of 1 liter after autoclaving. After 20 hours, a layer of thiostrepton (50 μg/ml final concentration) was plated on the top of the plates.

### Example 5

### Cloning and Expression of CALA from Malassezia furfur, Aspergillus sp., Fusarium sp., Kurtzmanomyces sp., Pichia stipitis, and Debaryomyces hansenii in Trichoderma reesei

Expression vectors for expressing CALA from Malassezia furfur, Aspergillus sp., Fusarium sp., Kurtzmanomyces sp., Pichia stipis, and Debaryomyces hansenii in Trichoderma reesei were made by recombining GATEWAY® entry vector pDONR 221 (Invitrogen, Corp. Carlsbad, Calif., USA) containing synthetic genes separately encoding each of the CALA with the T. reesei GATEWAY® destination vector pTrex3G (U.S. Pat. No. 7,413,879).

The vector pTrex3 g is based on the E. coli vector pSL1180 (Pharmacia, Inc., Piscataway, N.J., USA) which is a pUC118 phagemid-based vector (Brosius, J. (1989), DNA 8:759) with an extended multiple cloning site containing 64 hexamer restriction enzyme recognition sequences. This plasmid was designed as a Gateway destination vector (Hartley et al. (2000) Genome Research 10:1788-95) to allow insertion using Gateway technology (Invitrogen) of a desired open reading frame between the promoter and terminator regions of the T. reesei cbh1 gene. It also contains the Aspergillus nidulans amdS gene for use as a selective marker in transformation of T. reesei. The pTrex3 g is 10.3 kb in size and inserted into the polylinker region of pSL1180 are the following segments of DNA: a) a 2.2 by segment of DNA from the promoter region of the T. reesei cbh1 gene; b) the 1.7 kb Gateway reading frame A cassette acquired from Invitrogen that includes the attR1 and attR2 recombination sites at either end flanking the chloramphenicol resistance gene (CmR) and the ccdB gene; c) a 336 by segment of DNA from the terminator region of the T. reesei cbh1 gene; and d) a 2.7 kb fragment of DNA containing the Aspergillus nidulans amdS gene with its native promoter and terminator regions.

Expression vectors based on pKB483 (FIG. 5) and separately containing each of CALA of interest was transformed into a T. reesei host strain derived from RL-P37 (IA52) and having various gene deletions (Δcbh1, Δcbh2, Δeg1, Δeg2) using electroporation and biolistic transformation (particle bombardment using the PDS-1000 Helium system, BioRad Cat. No 165-02257) methods. The protocols are outlined below and reference is also made to Examples 6 and 11 of WO 05/001036.

### Transformation by Electroporation was Performed as Follows:

The T. reesei host strain was grown to full sporulation on PDA plates (BD Difco Potato Dextrose Agar, 39 g per liter in water) for 5 days at 28° C. Spores from 2 plates were harvested with 1.2 M sorbitol and filtered through miracloth to separate the agar. Spores were washed 5-6 times with 50 ml water by centrifugation. The spores were resuspended in a small volume of 1.2 M sorbitol solution. 90 μL of spore suspension was aliqouted into the electroporation cuvette (E-shot, 0.1 cm standard electroporation cuvette from Invitrogen, Carlsbad, Calif., USA). 1 μg/μL plasmid DNA was added to the spore suspension and electroporation was set at 16 kV/cm, 25 μF, 50Ω. After electroporation, the spore suspension was resuspended in 5 parts 1.0 M sorbitol and 1 part YEPD (BD Bacto Peptone 20 g, BD Bacto Yeast Extract 10 g with milliQ H₂O in 960 mL, with 40 mL 50% glucose added post sterilization), and allowed to germinate by overnight incubation at 28° C. with shaking at 250 rpm. Germlings were plated on minimal medium acetamide plates having the following composition: 0.6 g/L acetamide; 1.68 g/LCsCl; 20 g/L glucose; 20 g/L KH₂PO₄; 0.6 g/L CaCl₂.2H₂O; 1 ml/L 1000×trace elements solution; 20 g/L Noble agar; and pH 5.5. 1000×trace elements solution contained 5.0 g/L FeSO₄.7H₂O; 1.6 g/L MnSO₄; 1.4 g/L ZnSO₄.7H₂O and 1.0 g/L CoCl₂ 6H₂O).

Transformants were picked and transferred individually to acetamide agar plates. After 5 days of growth on minimal medium acetamide plates, transformants displaying stable morphology were inoculated into 200 μL Glucose/Sophorose defined media in 96-well microtiter plates. The microtiter plate was incubated in an oxygen growth chamber at 28° C. for 5 days. Supernatants from these cultures were used to assay for lipase activity using the spot assay.

Glucose/Sophorose defined medium (per liter) consists of (NH₄)₂SO₄, 5 g; PIPPS buffer, 33 g; Casamino Acids, 9 g; KH₂PO₄, 4.5 g; CaCl₂ (anhydrous), 1 g, MgSO₄.7H₂O, 1 g; pH 5.50 adjusted with 50% NaOH with sufficient milli-Q H₂O to bring to 966.5 mL. After sterilization, the following were added: 5 mL Mazu, 26 mL 60% Glucose/Sophrose, and 400×T. reesei Trace Metals 2.5 mL.

### Biolistic Transformation was Performed as Follows:

A suspension of spores (approximately 5×10⁸ spores/ml) from the T. reesei host strain was prepared. 100-200 μL of spore suspension was spread onto the center of plates containing minimal medium acetamide. The spore suspension was allowed to dry on the surface of the plates. Transformation followed the manufacturer's protocol. Briefly, 1 mL ethanol was added to 60 mg of M10 tungsten particles in a microcentrifuge tube and the suspension was allowed to stand for 15 seconds. The particles were centrifuged at 15,000 rpm for 15 seconds. The ethanol was removed and the particles were washed three times with sterile H₂O before 1 mL of 50% (v/v) sterile glycerol was added to them. 25 μL of tungsten particle suspension was placed into a microtrifuge tube. While continuously vortexing, the following were added: 5 μL (100-200 ng/μL) of plasmid DNA, 25 μL of 2.5M CaCl₂ and 10 μL of 0.1 M spermidine. The particles were centrifuged for 3 seconds.

The supernatant was removed and the particles were washed with 200 μL of 100% ethanol and centrifuged for 3 seconds. The supernatant was removed and 24 μL of 100% ethanol was added to the particles and mixed. Aliquots of 8 μL of particles were removed and placed onto the center of macrocarrier disks that were held in a desiccator. Once the tungsten/DNA solution had dried the macrocarrier disk was placed in the bombardment chamber along with the plate of minimal medium acetamide with spores and the bombardment process was performed according to the manufacturer's protocol. After bombardment of the plated spores with the tungsten/DNA particles, the plates were incubated at 30° C. Transformed colonies were transferred to fresh plates of minimal medium acetamide and incubated at 30° C. After 5 days of growth on minimal medium acetamide plates, transformants displaying stable morphology were inoculated into 20 mL Glucose/Sophorose defined media in shake flasks. The shake flasks were incubated in a shaker at 200 rpm at 28° C. for 3 days. Supernatants from these cultures were used to assay for lipase activity using the spot assay. For protein production, T. reesei transformants were cultured in fermenters as described in WO 2004/035070. Ultrafiltered concentrate (UFC) from tanks or ammonium sulfate purified protein samples were used for biochemical assays.

### Example 6

### Activity-Temperature Profile of CALA Sco-L

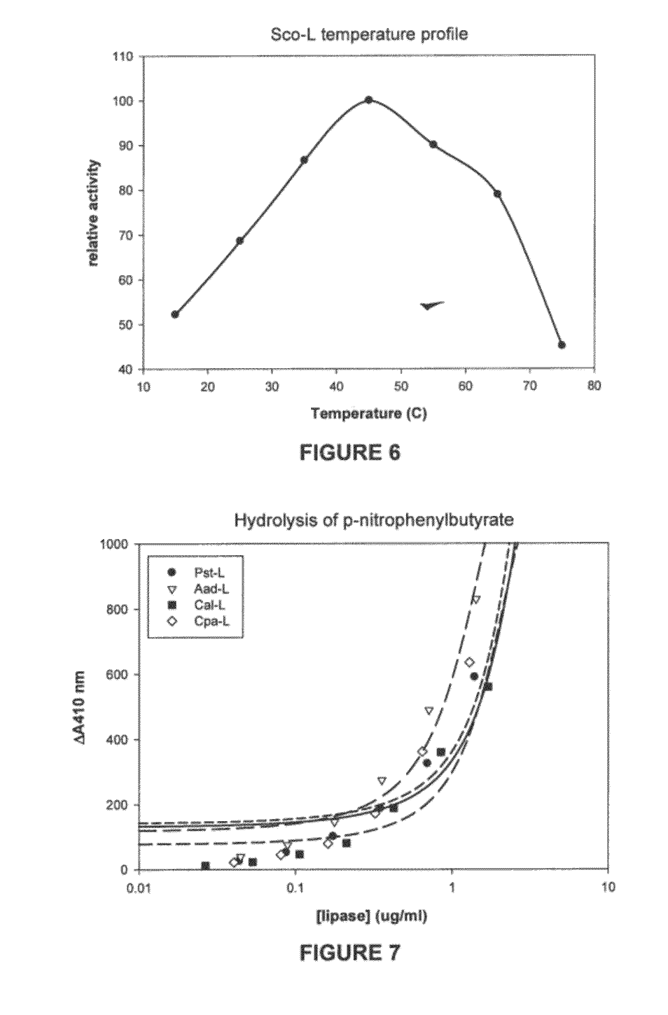

In this example, the effect of temperature on the activity of the CALA Sco-L was studied across a range of temperatures (15° C.-75° C.). CALA Sco-L activity at different temperatures was measured using the pNB hydrolysis assay as described in Example 1. As shown in FIG. 6, CALA Sco-L appeared to be most active at 45° C., which was considered the optimum temperature. Above 65° C. enzymatic activity declined abruptly.

### Example 7

### Stability of CALA Sco-L

### A. Stability in Detergent

This Example describes experiments performed to test the activity and stability of CALA Sco-L in commercially available detergents. A 5% (v/v) solution of purified CALA Sco-L (20 mg/ml) in a detergent composition (i.e., Laundry DROPPS, Cot'n Wash, Inc., Ardmore, Pa., USA) was incubated at room temperature. 10 μL of the resulting solution/suspension was removed at various time intervals over a period of 1 week, serially diluted, and tested for lipase activity as determined using the pNB assay described in Example 1. The residual enzyme activity was reported as a fraction of the activity measured at day 0 (Table 2).

TABLE 2
Stability of CALA Sco-L in DROPPS detergent

| Days in DROPPS detergent | 0 | 7 |
|---|---|---|
| Activity remaining | 1.00 | 0.68 |

### B. Stability in the Presence of Protease

This Example describes experiments performed to test the activity and stability of CALA Sco-L in the presence of protease. A 200 ppm stock solution of CALA Sco-L was prepared in 50 mM HEPES pH 8.2. 10 μL of serially diluted protease (B. amyloliquefaciens subtilisin BPN′-Y217L, Swissprot Accession Number P00782, BPN') in 50 mM HEPES pH 8.2 (protein concentration ranging from 0.1 to 100 ppm) was added to 100 μL of CALA Sco-L in 96-well microtiter plates. The plates were incubated for 30 min at 30° C. Residual lipase activity was measured with the pNB assay as described in Example 1. Relative lipase activity was calculated by normalizing the rate of hydrolysis of pNB to that of the zero timepoint. As shown in Table 3, CALA Sco-L activity is increased in the presence of protease, which appears to enhance its stability.

TABLE 3
Stability of Sco-L in the presence of BPN′ protease

| BPN′ Protease (ppm) | 0 | 5 | 21 |
|---|---|---|---|
| Activity remaining | 1.00 | 0.97 | 1.00 |

### Example 8

### Measurement of CALA Hydrolytic Activity

In this example, the ability of CALA to hydrolyse a variety of substrates (synthetic substrates, triglycerides, phospholipids, and lysophospholipids) was tested, using assays described in Example 1.

A. Hydrolysis of p-nitrophenyl esters of Various Chain Lengths by CALA

10 μL of serially diluted enzyme samples were incubated with 100 μL of substrate in reaction buffers described in Example 1. The release of p-nitrophenylate product was kinetically measured using the assay described in Example 1.

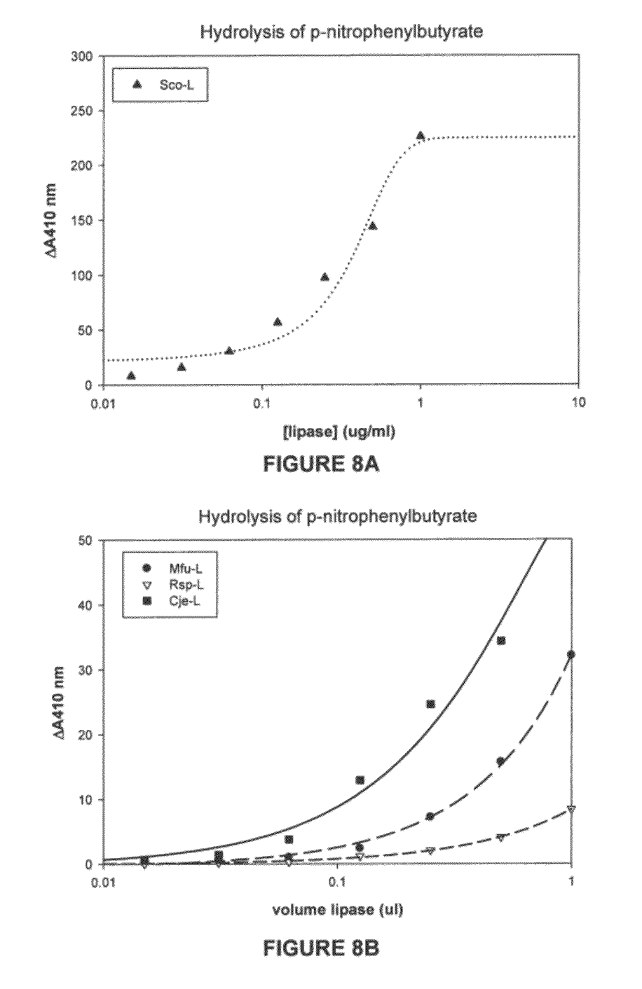

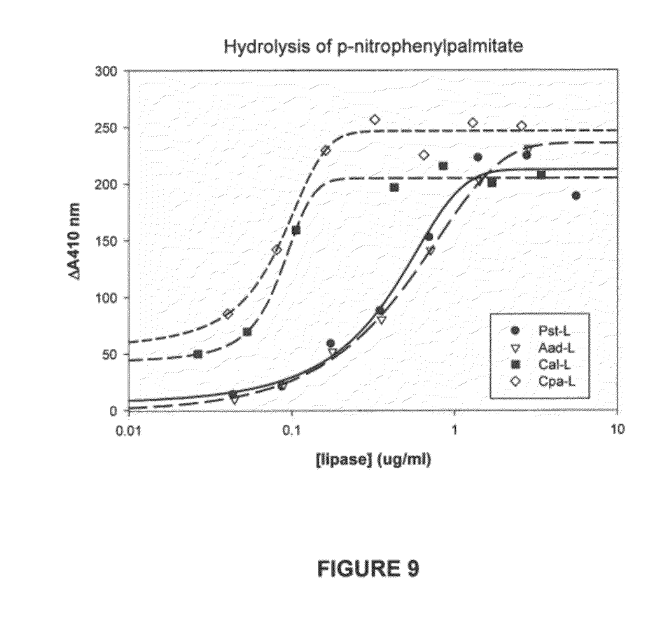

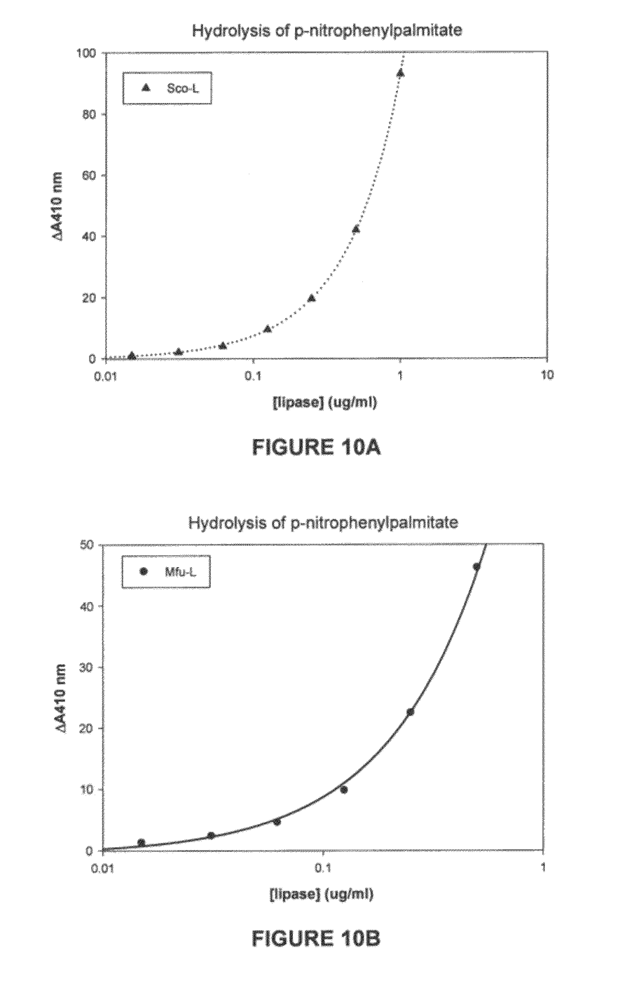

Hydrolysis of pNB substrate by CALA Cal-L, Cpa-L, Aad-L, and Pst-L is shown in FIG. 7. Hydrolysis of pNB substrate by CALA Sco-L, Cje-L, Rsp-L and Mfu-L is shown in FIGS. 8A and 8B. Hydrolysis of pNPP substrate by CALA Cal-L, Cpa-L, Aad-L, and Pst-L is shown in FIG. 9. Hydrolysis of pNPP substrate by CALA Sco-L and Mfu-L enzymes is shown in FIGS. 10A and 10B.

Chain-length preferences of CALA were determined by measuring the hydrolysis rate of substrates having different chain-lengths (i.e., C4, C8, C10, C16, and C18). For each CALA, the rate of product release was normalized to substrate with the highest activity. The results are shown in Table 4. Note that these data are influenced by the relative solubility of the substrates, which varies according to their chain-length and other structural features.

TABLE 4
Chain-length preference of CALA

| | Substrate | | | | |
|---|---|---|---|---|---|
| | p-nitrophenyl | p-nitrophenyl | p-nitrophenyl | p-nitrophenyl | p-nitrophenyl |
| | butyrate | octanoate | decanoate | palmitate | stearate |
| Enzyme | (C4: 0) | (C8: 0) | (C10: 0) | (C16: 0) | (C18: 0) |
| LIPOMAX | 0.23 | 0.63 | 1.00 | 0.53 | 0.27 |
| Pst-L | 0.20 | 0.23 | 1.00 | 0.43 | 0.23 |
| Cal-L | 0.53 | 0.83 | 1.00 | 0.33 | 0.31 |
| Cpa-L | 0.49 | 1.00 | 0.44 | 0.72 | 0.61 |
| Mfu-L | 0.74 | 1.00 | 0.85 | 0.58 | 0.35 |
| Aad-L | 0.25 | 0.31 | 1.00 | 0.62 | 0.47 |
| Sco-L | 0.28 | 0.03 | 0.23 | 1.00 | 0.59 |

### B. Hydrolysis of Triglycerides by CALA

10 μL aliquots of serially-diluted enzyme samples were incubated with trioctanoate (0.75%) in a 2% gum arabic emulsion in the buffer containing 50 mM HEPES, pH 8.2, 6 gpg, 2% PVA at 40° C., 450 rpm for 2 hours. The release of products was measured to determine triglyceride hydrolysis activity of CALA, using the 96-well microtiter plate-lipase activity assay described in Example 1. Hydrolysis of trioctanoate by CALA is shown in Table 5. Enzyme activity was reported relative to the activity of Pseudomonas alcaligenes variant M21L lipase (LIPOMAX™, Genencor, International, Palo Alto, Calif., USA), which was used as control. As above, the relative amount of activity is indicated by the number of “+;” n/d indicates that a value was not determined CALA Sco-L also showed hydrolysis activity using cholesteryl linoleate, phophatidylcholine, Tween-80, ethyl oleate, and ethyl palmitate as substrates (not shown). Note that the protein concentration was unknown in this experiment.

TABLE 5
Hydrolysis of trioctanoate in emulsion

| Enzyme | Activity |
|---|---|
| LIPOMAX | +++ |
| Pst-L | ++ |
| Cal-L | +++ |
| Cpa-L | +++ |
| Mfu-L | ++ |
| Aad-L | + |
| Sco-L | +++ |
| Rsp-L | n/d |
| Cje-L | n/d |

### Example 9

### Hydrolysis of Triglycerides on Cloth by CALA

The ability to hydrolysis triglycerides on cloth provides a good indication of the cleaning performance of CALA. Aliquots of enzymes samples were tested for their ability to hydrolyse trioctanoate bound to cloth using the microswatches triglyceride hydrolysis assay described in Example 1. As in Example 8, CALA activity was reported in relation to activity of Pseudomonas alcaligenes variant M21L lipase (LIPOMAX™), which was used as control. Table 6 summarizes the results for the CALA tested, wherein relative activity is indicated by the number of “+” and n/d indicates that a value was not determined. Of the CALA tested, only Sco-L demonstrated activity in heat inactivated TIDE® Cold Water 2× (Proctor & Gamble) laundry detergent, which includes both non-ionic and ionic surfactants and more water than DROPPS (not shown).

TABLE 6
Hydrolysis of trioctanoate on cloth by CALA

Enzyme activity

| | | 0.1% |
|---|---|---|
| | | DROPPS |
| Enzyme | Buffer | detergent |
| LIPOMAX | +++ | ++ |
| Pst-L | +++ | ++ |
| Cal-L | ++ | + |
| Cpa-L | +++ | + |
| Mfu-L | + | − |
| Aad-L | ++ | + |
| Sco-L | +++ | ++ |
| Rsp-L | n/d | n/d |
| Cje-L | n/d | n/d |

### Example 10

### Measurement of CALA Acyltransferase Activity

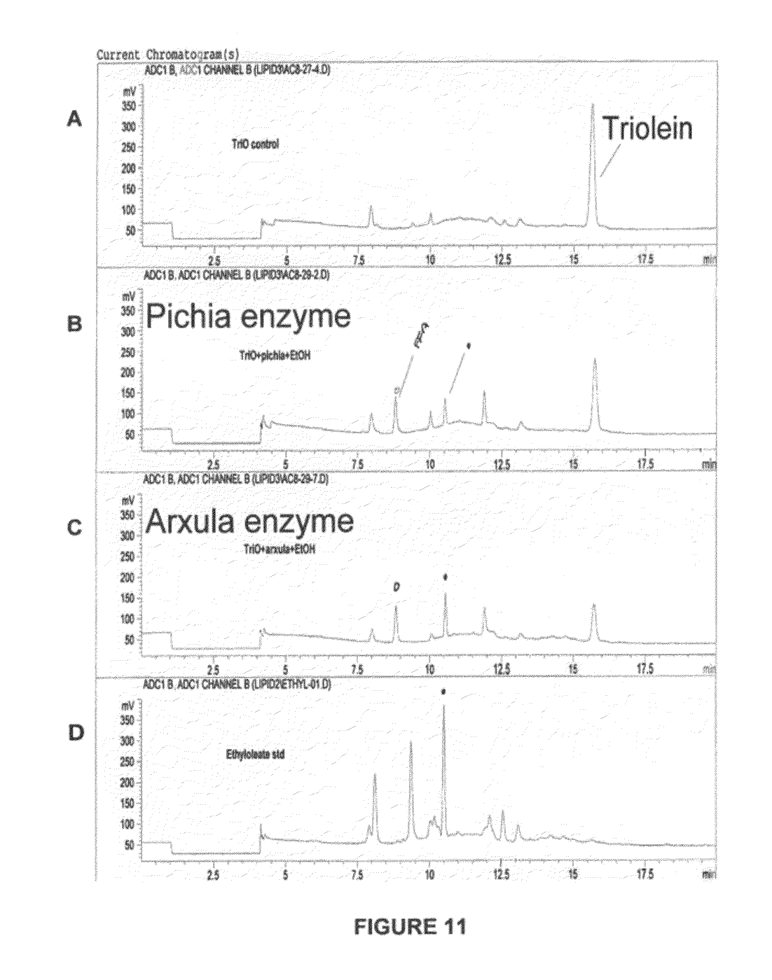

In this example, the ability of CALA Aad-L and Pst-L to perform a transesterification reaction in solution was tested using LC/MS analysis. Briefly, 20 μL of 20 g/L triolein in 4% gum arabic emulsion was added to 50 mM phosphate buffer at pH 6 or 8 in 96-well microtiter plates. 8% (v/v) ethanol or n-propanol acceptors were added to each well. 5 μL aliquots of purified enzyme or culture filtrate were then added to appropriate wells and the plate incubated at 30° C. for 4 hours. After incubation, 100 μL of the supernatant was added to 900 μL of acetone in a microfuge tube and the contents spun in a microcentrifuge. The resulting supernantant was diluted 3-fold into acetone and 30 μL was analyzed by LC/MS charged aerosol detection (LC/MS CAD) analysis. The results are shown in FIGS. 11A-11D. FIG. 11A shows the LC/MS profile of a control triolein sample with no added enzyme. FIGS. 11B and 11C shown the products of triolein hydrolysis produced by CALA Pst-L and Aad-L, respectively. FIG. 11D shows an ethyl oleate standard.

### Example 11

### Peracetic Acid Generation by CALA

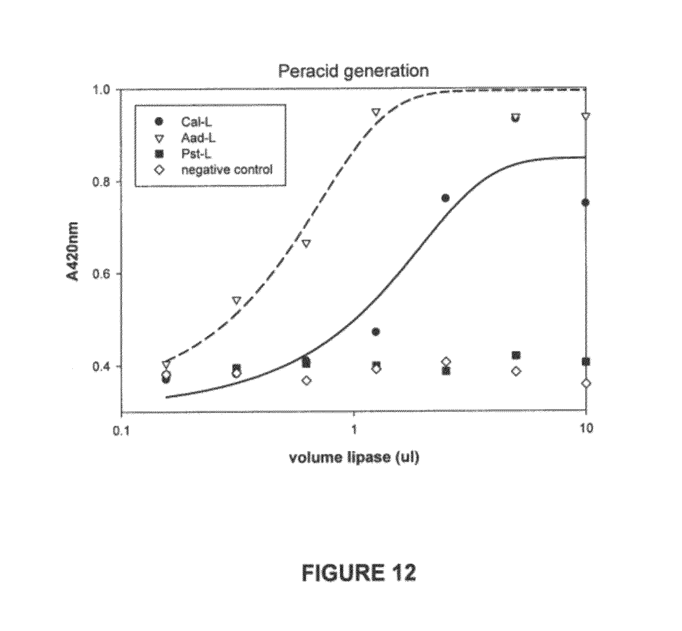

In this example, ultrafiltered concentrates of CALA Aad-L, Pst-L and Cal-L were assayed for their ability to generate peracetic acid using trioctanote as a donor and H₂O₂ as an acceptor. Potassium phosphate buffer (pH 8.0) was prepared using standard methods. The reaction buffer consisted of 2% (w/v, final concentration) poly(vinyl) alcohol (PVA; Sigma 341584) in 50 mM potassium phosphate solution buffered to pH 8.0. The substrate donor for the acyltransferase reaction was trioctanoate (Sigma T9126) and was added to the 2% PVA solution to a final concentration of 0.75% (v/v). Emulsions were prepared by sonicating the trioctanote in the PVA solutions for at least 20 minutes. Following formation of the emulsion, the acceptor, H₂O₂ (Sigma 516813), was added to the emulsions at a final concentration of 1% (v/v) H₂O₂. The negative control was a related enzyme with a preference for donor molecules with short chain (<4 carbons). Serial dilutions of CALA were incubated with the reaction buffer, which contained the emulsified donor and acceptor molecules buffered to pH 8, to 10% of the total volume of the reaction. The reactions were incubated for one hour at 25° C. Peracid generation was then assayed by mixing the reaction products (20% v/v) in a peracid detection solution consisting of 1 mM 2,2′-azino-bis(3-ethylbenzthiazoline-6-sulfonic acid) (ABTS; Sigma A-1888), 500 mM glacial acetic acid pH 2.3, and 50 μM potassium iodide. The reaction of peracids with ABTS resulted in the generation of a radical cation ABTS+ which has an absorbance maximum around 400-420 nm Peracid generation was assayed by measuring the absorbance of the reactions at 420 nm using a SpectraMax Plus384 microtiter plate reader. The results are shown in FIG. 12.

### Example 12

### Surfactant Generation by CALA as Determined by HPTLC Analysis

In this example, CALA Aad-L, Pst-L, and Cal-L were assayed by high performance thin layer chromatography (HPTLC) to measure the generation of surfactants using triolein, phosphatidylcholine (Avanti PC), sorbitan, or DGDG as donors, and 1,2-propanediol, 1,3-propanediol, 2-methyl-1-propanol(isobutylalcohol), sorbitan sorbitol, serine, ethanolamine, 1,2-ethylene glycol, polyglycerol, glucosamine, chitosan oligomer, maltose, sucrose, or glucose as acceptors.

140 μL of enzyme solution was added in a tube to 1 mL of substrate solution containing 2% donor substrates emulsified in 4% gum arabic and 0.8 g of acceptor in 50 mM phosphate buffer pH 6.0. The reactions were incubated at 30° C. for 1-4 hours. After incubation, 2 mL hexane:isopropanol solution (3:2) was added to the reaction and the tubes vortexed for 10 min. The organic phase was transferred to a new tube and 10 μL of the reaction products were used for HPTLC analysis.

Briefly, TLC plates (20×10 cm, Merck #1.05641) were activated by drying (160° C., 20-30 minutes). 10 μL of reaction products obtained as described above were applied to the TLC plate using an Automatic HPTLC Applicator (ATS4 CAMAG, Switzerland). Plate elution was performed using CHCl₃:Methanol:Water (64:26:4) for 20 minutes in a 7 cm automatic developing chamber (ADC2, CAMAG, Switzerland). After elution, plates were dried (160° C., 10 minutes), cooled, and immersed (10 seconds) in developing fluid (6% cupric acetate in 16% H₃PO₄). After drying (160° C., 6 minutes) plates were evaluated visually using a TLC visualizer (TLC Scanner 3 CAMAG, Switzerland). The results are shown in Table 7.

TABLE 7
Surfactant generation by Cal-L, Aad-L, and
Sco-L enzymes measured by HPTLC analysis

| Substrate | Acceptor | Enzyme | Activity |
|---|---|---|---|
| Triolein | 1,2 propanediol | Cal-L | + |
| Triolein | 1,2 propanediol | Cal-L | + |
| Triolein | 1,2 propanediol | Aad-L | + |
| Triolein | Sorbitan | Sco-L | + |

Endoglycosidase H (Endo H) (2.0 mg/mL) treated Cal-L performed comparably to untreated enzyme suggesting that glycosylation is neither required nor detrimental to enzyme activity.

### Example 13

### Surfactant Production as Measured by HPLC Analysis

In this example, Aad-L, Pst-L, Sco-L, and Cal-L were assayed by HPLC for generation of an ester surfactant using triolein as donor and 1,3-propanediol as acceptor.

For assaying activity, 5 μL of crude culture supernatant from fermentation media was added to 20 μL of emulsified substrate solution (stock: 20 g/l triolein in 4% gum arabic), 8% v/v of acceptor in 50 mM Phosphate buffer pH 6.0 or pH 8.0. The reactions were incubated overnight at 30° C. After incubation, 100 μL of the supernatant was added to 900 μL of acetone in a microfuge tube and the contents spun in a microcentrifuge. After centrifugation, the supernantant was transferred to a fresh tube and further diluted 3-fold with acetone, and 30 μL of this diluted supernatant was analyzed by LC/MS CAD (charged aerosol detection) analysis as described below.

An Agilent 1100 (Hewlett Packard) HPLC was equipped with Alltima HP C18 column (250×4.6 mm; Grace Davison). Compounds were eluted using a gradient beginning with solvent A (97% acetonitrile and 0.5% formic acid) with linearly increasing amounts of solvent B (neat acetone) over 10 minutes, followed by an isocratic phase in solvent B. The HPLC system was interfaced to an ABI 3200 QTrap MS (run under APCI mode), and a charged aerosol detector (ESA Biosciences) was used for quantification. LC/MS CAD analysis (Table 8) showed the formation of propylene glycol ester of fatty acids

TABLE 8
Surfactant generation by Cal-L, Cpa-L, Aad-L, Pst-L, Sco-L,
Cje-L, Mfu-L, and Rsp-L enzymes by HPLC analysis

| Substrate | Acceptor | Enzyme | Activity |
|---|---|---|---|
| Triolein | 1,3 propanediol | Cal-L | + |
| Triolein | 1,3 propanediol | Cpa-L | + |
| Triolein | 1,3 propanediol | Aad-L | + |
| Triolein | 1,3 propanediol | Pst-L | + |
| Triolein | 1,3 propanediol | Sco-L | + |
| Triolein | 1,3 propanediol | Cje-L | +/− |
| Triolein | 1,3 propanediol | Mfu-L | + |
| Triolein | 1,3 propanediol | Rsp-L | − |

### Example 14

### Biodiesel Generation as Measured by HPLC Analysis

In this example, Aad-L and Pst-L enzymes were assayed for their ability to perform a synthetic reaction using triolein as the acyl donor and methanol or ethanol as the acyl acceptor.

For assaying activity, 20 μL of a 20 g/L triolein in 4% gum arabic emulsion was added to 50 mM phosphate buffer at pH 6 or 8 in 96-well microtiter plates. 8% (v/v) of acceptor (methanol or ethanol) was added to each well. Appropriately diluted enzyme solution was added to the wells and the plate was incubated overnight at 30° C., with continuous mixing. After incubation, 100 μL of the supernatant was added to 900 μL of acetone in a microfuge tube and the contents spun in a microcentrifuge. After centrifugation, the supernantant was transferred to a fresh tube and further diluted 3-fold with acetone, and 30 μL of this diluted supernatant was analyzed by LC/MS CAD (charged aerosol detection) analysis as described below.

An Agilent 1100 (Hewlett Packard) HPLC was equipped with Alltima HP C18 column (250×4.6 mm; Grace Davison). Compounds were eluted using a gradient beginning with solvent A (97% acetonitrile and 0.5% formic acid) with linearly increasing amounts of solvent B (neat acetone) over 10 minutes, followed by an isocratic phase in solvent B. The HPLC system was interfaced to an ABI 3200 QTrap MS (run under APCI mode), and a charged aerosol detector (ESA Biosciences) was used for quantification. Biodiesel consisting of fatty acid methyl and ethyl esters were formed (FIGS. 13A and 13B).

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A recombinant lipase/acyltransferase enzyme having only limited amino acid sequence identity to Candida albicans Cal-L lipase/acyltransferase, comprising:
a) a first amino acid sequence motif GX₁SX₂G at residues corresponding to positions 192-196 of the Cpa-L amino acid sequence (SEQ ID NO: 8), where X₁ is an aromatic amino acid and X₂ is an amino acid selected from the group consisting of G, E, or Q;
b) a second amino acid sequence motif YAX₁X₂X₃, at residues corresponding to positions 210-214 of the Cpa-L amino acid sequence (SEQ ID NO: 8), where X₁ is P or K, X₂ is an acidic amino acid, and X₃ is a non-polar aliphatic amino acid;
c) lipase/esterase activity based on hydrolysis of p-nitrophenylbutyrate in an aqueous solution.

2. The lipase/acyltransferase enzyme of claim 1, having less than about 50% amino acid sequence identity to Cal-L lipase/acyltransferase having the amino acid sequence of SEQ ID NO: 8.

3. The lipase/acyltransferase enzyme of claim 1, having a precursor amino acid sequence of at least 390 amino acid residues.

4. The lipase/acyltransferase enzyme of claim 1, wherein X₁ in the first amino acid sequence motif is selected from the group consisting of Y and H.

5. The lipase/acyltransferase enzyme of claim 1, wherein X₂ in the first amino acid sequence motif is selected from the group consisting of G and Q.

6. The lipase/acyltransferase enzyme of claim 1, wherein the first amino acid sequence motif has a sequence selected from the group consisting of GYSGG, GYSQG, and GHSQG.

7. The lipase/acyltransferase enzyme of claim 1, wherein X₁ in the second amino acid sequence motif is selected from the group consisting of D and E.

8. The lipase/acyltransferase enzyme of claim 1, wherein X₂ in the second amino acid sequence motif is selected from the group consisting of L, V, and I.

9. The lipase/acyltransferase enzyme of claim 1, wherein the second amino acid sequence motif has a sequence selected from the group consisting of YAPEL, YAPDV, YAPDL, YAPEI, and YAKEL.

10. The lipase/acyltransferase enzyme of claim 1, having an amino acid sequence having at least 90% identity to an amino acid sequence selected from the group consisting of SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53.

11. The lipase/acyltransferase enzyme of claim 1, with the provisio that the lipase/acyltransferase enzyme does not have the amino acid sequence of SEQ ID NO: 5 or SEQ ID NO: 8.

12. The lipase/acyltransferase enzyme of claim 1, wherein the lipase/acyltransferase enzyme is selected from the group consisting of Aad-L, Pst-L, Sco-L, Mfu-L, Rsp-L, Cje-L, Ate-L, Aor-L-0488, Afu-L, Ani-L, Acl-L, Aor-L-6767, Fve-L, Fgr-L, Ksp-L, and Dha-L.

13. The lipase/acyltransferase enzyme of claim 1, with the provisio that the lipase/acyltransferase enzyme is not Cal-L or CpaL.

14. A recombinant lipase/acyltransferase enzyme having at least 90% amino acid sequence identity to an amino acid sequence selected from the group consisting of SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53.

15. A composition comprising the lipase/acyltransferase enzyme of claim 1.

16. The composition of claim 14, wherein the lipase/acyltransferase enzyme is expressed in a heterologous host cell.

17. The composition of claim 15, wherein the composition is a detergent composition, and the lipase/acyltransferase enzyme is Sco-L.

18. A composition comprising a recombinant lipase/acyltransferase enzyme having at least 90% amino acid sequence identity to an amino acid sequence selected from the group consisting of SEQ ID NO: 2, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53.

19. A method for removing an oily soil or stain from a surface, comprising contacting the surface with a composition comprising the lipase/acyltransferase enzyme of claim 1.

20. The method of claim 19, wherein the composition is a detergent composition and the lipase/acyltransferase is Sco-L.

21. The method of claim 19, wherein the surface is a textile surface.

22. A method for forming a peracid comprising contacting an acyl donor and hydrogen peroxide with the lipase/acyltransferase enzyme of claim 1.

23. The method of claim 22, wherein the lipase/acyltransferase enzyme is Aad-L.

24. A method for forming an ester surfactant comprising contacting an acyl donor and acceptor with the lipase/acyltransferase enzyme of claim 1.

25. The method of claim 24, wherein the lipase/acyltransferase enzyme is Aad-L, Pst-L, Sco-L, or Mfu-L.

26. A method for making biodiesel, comprising contacting an acyl donor and acceptor with the lipase/acyltransferase enzyme of claim 1.

27. The method of claim 26, wherein the lipase/acyltransferase enzyme is Aad-L or Pst-L.

28. (canceled)

29. An expression vector comprising a polynucleotide encoding the lipase/acyltransferase enzyme of claim 1 and a signal sequence to cause secretion of the lipase/acyltransferase enzyme.

30. An expression vector comprising a polynucleotide encoding the lipase/acyltransferase enzyme Cal-L or Cpa-L and a signal sequence to cause secretion of the lipase/acyltransferase enzyme.

31. A method for expressing a lipase/acyltransferase enzyme, comprising:
introducing the expression vector of claim 29 into a suitable host,
expressing the lipase/acyltransferase enzyme, and
recovering the lipase/acyltransferase enzyme expressed.

32. A method for expressing a lipase/acyltransferase enzyme, comprising:
introducing the expression vector of claim 30 into a suitable host,
expressing the lipase/acyltransferase enzyme, and
recovering the lipase/acyltransferase enzyme expressed.

33. The method of claim 19, wherein the lipase/acyltransferase enzyme is expressed in a heterologous host cell.

34. The method of claim 22, wherein the lipase/acyltransferase enzyme is expressed in a heterologous host cell.

35. The method of claim 24, wherein the lipase/acyltransferase enzyme is expressed in a heterologous host cell.

36. The method of claim 26, wherein the lipase/acyltransferase enzyme is expressed in a heterologous host cell.


SEQUENCE LISTING (68 sequences)

SEQ ID NO: 1 - protein, 448 aa, Arxula adeninivorans
MLKLLLLVAQLVAFVFGAALPEERVYARDAVTPPSEDPFYTPDDGYEKEEPGTILRWRHAPQMPAFTVFKQNLNASIQILYRTTDTQGQPTATVVTVMIPHNHAEGKLLSYQSWEDSAWINCAPSYAIQFGANPQGIISQVDMLTLQSALNEGWIVSTPDYEGPKSSFTSAIISGQATLDSIRAVLKSESFTGVKPDSKVAMWGYSGGSIASGWAAALQPTYAPELKIAGAALGGVIQNITSVAVQVNKGPFVGLVPAGIKGLSSQYPELEDYINDQLLPDKRDDFEKAGKQCLSVDVLTYAFQDWFSYTKAGDRVLYNETIQKVLDENAMGKQKPQIPLLFYHGVHDEVMPIADVDKLYYEYCSNGVTVEYYREEGSEHVLEMITGFPKAYNYVKNLLDGGSVSSGCQRHDVFSNAFDEDALPTYSAEIWGILKGLLGAPVGPAAIS

SEQ ID NO: 2 - protein, 431 aa, Arxula adeninivorans
AALPEERVYARDAVTPPSEDPFYTPDDGYEKEEPGTILRWRHAPQMPAFTVFKQNLNASIQILYRTTDTQGQPTATVVTVMIPHNHAEGKLLSYQSWEDSAWINCAPSYAIQFGANPQGIISQVDMLTLQSALNEGWIVSTPDYEGPKSSFTSAIISGQATLDSIRAVLKSESFTGVKPDSKVAMWGYSGGSIASGWAAALQPTYAPELKIAGAALGGVIQNITSVAVQVNKGPFVGLVPAGIKGLSSQYPELEDYINDQLLPDKRDDFEKAGKQCLSVDVLTYAFQDWFSYTKAGDRVLYNETIQKVLDENAMGKQKPQIPLLFYHGVHDEVMPIADVDKLYYEYCSNGVTVEYYREEGSEHVLEMITGFPKAYNYVKNLLDGGSVSSGCQRHDVFSNAFDEDALPTYSAEIWGILKGLLGAPVGPAAIS

SEQ ID NO: 3 - DNA, 1554 bp, Artificial Sequence
  Synthetic DNA
atgagattcccgtcgattttcaccgctgttctgttcgctgcttcttctgctttggccgctccagttaacactactaccgaggacgagactgctcaaattccagccgaggccgttattggttactctgacttggagggcgactttgacgttgctgtgctgccattctcgaactctaccaacaacggcctgctgttcatcaacaccaccattgcctctattgccgccaaagaggaaggcgtttccttggagaagagagctgctttgccagaggaaagagtgtacgccagagatgctgttactccaccatctgaggacccattctacactccagacgacggttacgagaaagaggaaccaggaaccatcttgagatggagacacgctccacagatgccagctttcaccgtgtttaagcagaacctgaacgcctctatccagatcctgtacagaaccactgacactcagggtcaaccaaccgctaccgtggtgaccgttatgattccacacaaccacgctgagggaaagttgctgtcgtaccagtcttgggaagattctgcctggattaactgcgctccatcttacgctattcagttcggtgctaacccacaaggtatcatctctcaggtggacatgttgaccttgcagtctgctttgaacgagggatggatcgtttctactccagactacgagggtccaaagtcgtctttcacctcggccatcatttctggacaggctaccctggactctattagagccgtgctgaagtctgagtctttcaccggtgttaagccagactctaaggttgcaatgtggggctactctggtggttctattgcttctggatgggctgctgctttgcaaccaacttacgccccagagctgaaaattgctggtgctgctctgggtggtgttatccagaacatcacctctgttgccgtgcaggttaacaagggaccatttgtgggattggttccagccggtatcaagggattgtcctcgcaatacccagaattggaggactacatcaacgaccagctgttgccagacaagagagatgacttcgagaaggccggaaagcagtgtttgtctgtggacgttctgacctacgctttccaggactggttctcttacactaaggccggtgacagagttttgtacaacgagactatccagaaggttctggacgaaaacgctatgggaaagcagaagccacagatcccactgttgttctaccacggtgttcacgacgaggttatgccaattgccgacgtggacaagttgtactacgagtactgctctaacggtgttaccgtggagtactacagagaggaaggttctgagcacgtgttggagatgattaccggtttcccaaaggcctacaactacgtgaagaacctgttggacggtggctctgtttcttctggttgccagagacacgacgttttctctaacgctttcgacgaggacgctctgccaacttactctgccgagatctggggaattttgaagggtctgttgggtgctccagttggtccagccgccatctcgtaataa

SEQ ID NO: 4 - protein, 459 aa, Candida albicans
MLFLLFLLVAPIYAGLILPTKPSNDPFYNAPAGFEKAAVGEILQSRKTPKPITGVFVPVKIQNSWQLLVRSEDSFGNPNVIVTTVMEPFNADPSKLASYQVFEDSAKADCAPSYALQFGSDVTTIATQVETYLLAPLLDQGYYVVSPDYEGPKSTFTVGKQSGQAVLNSIRAALKSGKITNLAEDAKVVMWGYSGGSLASGWAAALQPNYAPELGGNLLGAALGGFVTNITATAEATDGTVFAGIMANALGGVANEYPEFKQILQNDTDKQSVFDQFDNHCLADGVINYIGKHFLSGTNKIFKSGWNILKNPTISKIVEDNGLVYQKQLVPKIPILIYHGAIDQIVPIVNVKKTYQNWCDAGIASLEFSEDATNGHITETIVGAPVALTWIINRFNGKQTVSGCQHVKRTSNFEYPNIPPSILNYFKAALNILIQKGLGPDIQKDQVNPDGLKKIPILV

SEQ ID NO: 5 - protein, 444 aa, Candida albicans
LILPTKPSNDPFYNAPAGFEKAAVGEILQSRKTPKPITGVFVPVKIQNSWQLLVRSEDSFGNPNVIVTTVMEPFNADPSKLASYQVFEDSAKADCAPSYALQFGSDVTTIATQVETYLLAPLLDQGYYVVSPDYEGPKSTFTVGKQSGQAVLNSIRAALKSGKITNLAEDAKVVMWGYSGGSLASGWAAALQPNYAPELGGNLLGAALGGFVTNITATAEATDGTVFAGIMANALGGVANEYPEFKQILQNDTDKQSVFDQFDNHCLADGVINYIGKHFLSGTNKIFKSGWNILKNPTISKIVEDNGLVYQKQLVPKIPILIYHGAIDQIVPIVNVKKTYQNWCDAGIASLEFSEDATNGHITETIVGAPVALTWIINRFNGKQTVSGCQHVKRTSNFEYPNIPPSILNYFKAALNILIQKGLGPDIQKDQVNPDGLKKIPILV

SEQ ID NO: 6 - DNA, 1596 bp, Artificial Sequence
  Synthetic DNA
atgagattcccgtcgattttcaccgctgttctgttcgctgcttcttctgctttggccgctccagttaacactactaccgaggacgagactgctcagattccagccgaggccgttattggttactctgacttggagggcgactttgacgttgctgtgctgccattctcgaactctaccaacaacggcctgctgttcatcaacaccaccattgcctctattgccgctaaagaggaaggcgtttccttggagaagagaggactgattctgccaaccaagccatctaacgacccattctacaacgctccagccggttttgagaaagctgctgtgggagagattctgcagtctagaaagaccccaaagccaatcaccggtgtgttcgtgccagttaagatccagaactcgtggcagctgcttgttagatcggaggactctttcggtaacccaaacgtgatcgtgaccaccgttatggaaccttttaacgccgacccatctaagttggcctcttaccaggttttcgaggactctgctaaggctgactgtgctccatcttacgctctgcagttcggttctgacgttaccaccattgctacccaggttgagacttacctgctggctccattgttggaccagggatactacgttgtgtctccagactacgagggtccaaagtctaccttcactgtgggaaagcagtctggacaggccgttctgaactctattagagccgccctgaagtctggaaagatcaccaacttggccgaggacgccaaagttgttatgtggggctactctggtggttctttggcttctggatgggctgctgctttgcagccaaactacgctccagagctgggtggtaacttgttgggtgctgctctgggaggtttcgttaccaacattaccgctaccgctgaggctactgacggtactgttttcgccggtatcatggctaacgctctgggtggtgttgctaacgagtaccctgagttcaagcagattctgcagaacgacactgacaagcagagcgttttcgaccagttcgacaaccactgtttggccgacggtgttatcaactacatcggcaagcactttctgtctggcaccaacaagatcttcaagtcgggctggaacattctgaagaacccgaccatctctaagatcgtggaggacaacggattggtttaccagaagcagctggttccaaagatcccaatcctgatctaccacggtgccatcgaccagatcgtgccaatcgtgaacgtgaagaaaacctaccagaactggtgtgacgctggtattgcctctttggagttctctgaggacgcaaccaacggtcacatcaccgagactatcgttggtgctccagttgctctgacctggatcatcaacagattcaacggcaagcagaccgtttctggttgtcagcacgtgaagagaacctcgaacttcgagtacccaaacatcccaccatcgatcctgaactacttcaaggccgccctgaacattttgatccagaagggactgggtccagacattcagaaggaccaggttaacccagacggactgaagaagatcccgatcctggtttagtag

SEQ ID NO: 7 - protein, 465 aa, Candida parasilopsis
MRYFAIAFLLINTISAFVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAF

SEQ ID NO: 8 - protein, 448 aa, Candida parasilopsis
VLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAF

SEQ ID NO: 9 - DNA, 1248 bp, Artificial Sequence
  Synthetic DNA
atgagattcccgtcgattttcaccgctgttctgttcgctgcttcttctgctttggccgctccagttaacactactaccgaggacgagactgctcaaattccagccgaggccgttattggttactctgacttggagggcgactttgacgttgctgtgctgccattctcgaactctaccaacaacggcctgctgttcatcaacaccaccattgcctctattgccgccaaagaggaaggcgtttccttggagaagagattcgttctggccccaaagaaaccatctcaggacgacttttacactccaccacagggttacgaagctcaaccactgggttctatcctcaagaccagaaacgttccaaacccactgaccaacgtgtttaccccagtgaaggttcagaacgcttggcagctgcttgttagatctgaggacaccttcggtaacccaaacgccatcgtgaccaccattatccagccattcaacgccaagaaggacaagctggtttcgtaccagacctttgaggactctggaaagttggactgtgccccatcttacgctatccagtacggctctgacatttctaccctgaccacccagggtgaaatgtactacatctcggctttgttggaccagggatactacgttgttaccccagactacgagggtccaaagtctacctttactgtgggactgcagtctggtagagctaccctgaactctctgagagccaccttgaagtctggtaacctgaccggtgtttcttctgacgctgagactctgttgtggggatactctggtggttctctggcttctggatgggctgctgccattcagaaagagtacgccccagagctgtctaaaaacctgctgggtgctgctttgggaggtttcgtgaccaacattaccgctactgctgaggctgttgactctggtccattcgccggtatcatctctaatgccctggccggtattggtaacgagtacccggacttcaagaactacctgctgaagaaggtttcgccactgctgtctatcacctacagactgggcaacactcactgtttgctggacggtggtattgcttacttcggcaagtcgttcttctcgagaatcatcagatacttcccagacggatgggaccttgttaaccaagagccgatcaagaccattttgcaggacaacggactggtttaccagccaaaggacctgactccacagatcccactgttcatctaccacggtactctg

SEQ ID NO: 10 - protein, 482 aa, Pichia stipitis
MYFSQLYTIFLCLCIFHRASAAPQKVLKPTEDSFYDAPKGFEDAEIGTILKIRKTPHMLRSVYIPINVQNSWQILVRSESAEGNATAIVTTVIEPYNADPSKLVSYQVAEDSSSENCAVSYSLEFGASMDTIIAQVEMYFMQAALEQGYYVVTPDYEGPQASFTAGRQAGHAVLDSIRATLASSNVTGVDPDAEVVMWGYSGGSLASGWAAALQPKYAPELEGQILGAALGGFVTNITLTAVSVDDNIFAGLIASAINGLMNEYPEFSELAKDMIRPERLENFLKADSYCMVPSLIHYAFDNFFVGNDSYFTQGLDVFKIPFVQDMINSNTLALKNNTEIPQIPLFIYHGELDEIVPFSGSQRAYTNWCEWGIESLEFSTAMLSGHITEFFMGAPAALTWVIERFEGKQPVKGCQKTQRLTNLDYPGTPEALQIYFQAAYESVFQMDVGPNGENFTKSDLQRLAKRSFNGFTPIDTSNLKKR

SEQ ID NO: 11 - protein, 482 aa, Pichia stipitis
MYFSQLYTIFLCLCIFHRALAAPQKVLKPTEDLFYDAPKGFEDAEIGTILKIRKTPHMLRSVYIPINVQNSWQILVRSESAEGNATAIVTTVIEPYNADPSKLVSYQVAEDSSSENCAVSYSLEFGASMDTIIAQVEMYFMQAALEQGYYVVTPDYEGPQASFTAGRQAGHAVLDSIRATLASSNVTGVDPDAEVVMWGYSGGSLASGWAAALQPKYAPELEGQILGAALGGFVTNITLTAVSVDDNIFAGLIASAINGLMNEYPEFSELAKDMIRPERLENFLKADSYCMVPSLIHYAFDNFFVGNDSYFTQGLDVFKIPFVQDMINSNTLALKNNTEIPQIPLFIYHGELDEIVPFSGSQRAYTNWCEWGIESLEFSTAMLSGHITEFFMGAPAALTWVIERFEGKQPVKGCQKTQRLTNLDYPGTPEALQIYFQAAYESVFQMDVGPNGENFTKSDLQRLAKRSFNGFTPIDTSNLKKR

SEQ ID NO: 12 - DNA, 1452 bp, Artificial Sequence
  Synthetic DNA
atgtacttctcgcagctgtacaccatctttctgtgcctgtgcatcttccatagagctttggctgctccacagaaggttctgaagccaaccgaggacttgttttacgacgccccaaagggttttgaggacgccgagatcggtactatcctgaagatcagaaagaccccacacatgctgagatctgtgtacatcccaatcaacgtgcagaactcgtggcagatccttgtgagatctgagtctgctgagggtaacgctaccgctattgtgaccaccgttatcgagccatacaacgccgacccatctaagttggtgtcttaccaggttgcagaggactcttcttctgagaactgcgccgtgtcttactctttggagttcggcgcttctatggacaccatcattgcccaggtggagatgtactttatgcaggccgctttggagcaaggatactacgttgtgaccccagattacgaaggtcctcaggcttcttttaccgctggtagacaagctggtcacgctgttctggactctattagagccaccctggcctcttctaacgttactggcgttgacccagacgctgaagttgttatgtggggctactctggtggttctttggcttctggatgggctgctgctttgcagccaaaatacgccccagagttggagggtcaaattctgggtgctgctctgggaggtttcgttaccaacatcaccctgaccgctgtttctgttgacgacaacatcttcgccggactgattgcctctgctatcaacggactgatgaacgagtacccagagttttctgagctggccaaggacatgattagaccagagagactcgagaacttcctgaaggccgactcttactgtatggtgccatccctgatccactacgccttcgacaacttcttcgtgggtaacgactcttactttacccagggactggacgttttcaagatcccattcgtgcaggacatgatcaactctaacaccctggccctgaagaacaacactgagatcccacagatccctctgtttatctaccacggcgagctggatgagattgtgccattctcgggttctcagagagcttacaccaactggtgtgagtggggtattgagtccttggagttctctaccgctatgctgtctggtcacatcaccgagttcttcatgggtgctccagctgctttgacttgggtgatcgagagattcgagggaaagcagccagttaagggctgtcagaaaacccagagactgaccaacttggactacccaggtactccagaggctctgcagatctactttcaggccgcctacgagtctgttttccagatggatgtgggtccaaacggcgagaacttcaccaagtctgacctgcagagactggccaagagatctttcaacggcttcaccccaattgacacctcgaacctgaagaagagatagtag

SEQ ID NO: 13 - protein, 424 aa, Rhodococcus sp.
MKRHRFRAVTAAAVTLAAVAMFGSAGQVVAEPLTAAAAADFYLPPHPLPAGNSGDLIRSEPFRPLGASPLAALVPAASATRIMYLSSDTVGAPVAVTGTILEPTAAWPGSGPRPLVSYTYGTIGAGDNCAPSKLIADGITTDGAGTPMPNLYILDVVSLLARGITVVVTDYEGLGTPGAHTYLQPVPEAHAVLDATRAAIRFGAATPRAPVGIWGYSQGGSAAGGAAQLAHSYAPELNLAGTVVGAPPANVASTLSNVDGKALTDVIGFFLNGLLASHPEFTEAVTTMLNSDGIDFLHRTATECDVNALFHAYQPTSSLTVRGRPIIDELQAHPAIRAVLDSFNLGTPAPADPVLLARNVNDDVTPAADTYDLENAWRGAGADVTVAHLDTPPLLPQLGAAGHGVGLLLDNPTAVQWLIDRFNH

SEQ ID NO: 14 - protein, 387 aa, Rhodococcus sp.
AADFYLPPHPLPAGNSGDLIRSEPFRPLGASPLAALVPAASATRIMYLSSDTVGAPVAVTGTILEPTAAWPGSGPRPLVSYTYGTIGAGDNCAPSKLIADGITTDGAGTPMPNLYILDVVSLLARGITVVVTDYEGLGTPGAHTYLQPVPEAHAVLDATRAAIRFGAATPRAPVGIWGYSQGGSAAGGAAQLAHSYAPELNLAGTVVGAPPANVASTLSNVDGKALTDVIGFFLNGLLASHPEFTEAVTTMLNSDGIDFLHRTATECDVNALFHAYQPTSSLTVRGRPIIDELQAHPAIRAVLDSFNLGTPAPADPVLLARNVNDDVTPAADTYDLENAWRGAGADVTVAHLDTPPLLPQLGAAGHGVGLLLDNPTAVQWLIDRFNH

SEQ ID NO: 15 - DNA, 1302 bp, Artificial Sequence
  Synthetic DNA
atgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggccgccgcggacttctacctgccgccgcaccccctcccggcgggcaacagcggcgacctgatccgctcggagccgttccggccgctgggggcctcgccgctggccgcgctcgtcccggcggcctccgccacgcggatcatgtacctctcgtccgacacggtgggcgcccccgtcgcggtcaccggcacgatcctggagccgaccgccgcgtggccgggctccggcccgcggcccctggtgtcctacacctacggcaccatcggcgccggcgacaactgcgccccgtcgaagctcatcgcggacggcatcacgaccgacggggcgggcaccccgatgcccaacctgtacatcctggacgtcgtgtccctcctggcgcgcggcatcaccgtcgtggtcaccgactacgagggcctgggcacccccggcgcgcacacgtacctgcagccggtccccgaggcgcacgccgtcctcgacgcgacccgcgccgcgatccgcttcggggccgcgaccccgcgggcccccgtggggatctggggctactcgcagggcgggtccgccgcgggcggcgccgcgcagctggcgcacagctacgcccccgagctcaacctggccggcaccgtggtgggcgccccgcccgcgaacgtggcgagcacgctgtcgaacgtggacggcaaggccctcaccgacgtgatcggcttcttcctgaacggcctgctcgcctcccaccccgagttcacggaggccgtcaccacgatgctcaacagcgacggcatcgacttcctgcaccggacggccaccgagtgcgacgtcaacgccctgttccacgcctaccagccgaccagctccctgacggtccgcggccggcccatcatcgacgagctccaggcccaccccgcgatccgcgccgtcctggacagcttcaacctgggcaccccggcgccggccgacccggtgctgctcgcccgcaacgtcaacgacgacgtcaccccggcggccgacacgtacgacctggagaacgcctggcggggcgcgggggccgacgtgaccgtcgcccacctcgacaccccgcccctgctcccccagctgggcgccgcggggcacggcgtgggcctgctcctggacaaccccaccgccgtccagtggctgatcgaccggttcaaccactga

SEQ ID NO: 16 - protein, 445 aa, Streptomyces coelicolor
MPPRPRMLAAAITAALALGAQAVPAAAADGPAGDSTATTSRGVEIPAFYTPPSELPAADGTLIRHEPLPLALSLPGIDGPLPGRATRLMYKSTDANGEAVAVTGAYVEPAAKWRGDGPRPLVAVAPGTMGQGDQCAASMALEHPLQLNGQTVSVGYEDLSVYRLLLRGVAVVVTDYVGLGTTDRLHTYVNRVDGAHAVLDAVRAARSLDSASVTSDSRVGLFGYSQGGGATAAAAELQPSYAPDVQLAGTYAGAPPADLTEVTKAIDGSDLAGALGWSLNGFLQTEPALRPIADRYINEAGQEALKDLSTMCVGDALFGYGGDSSTDWTRTGQSISDVIRAEPALQSFLAEQRIGSTAPGSPVRVATGVSDDLVPHGQARRLAVDWCGKGGKVTYVPVLIPGVGSGLLNHFAPLLADQGNAIAWLTDRLSGEPAGSNCWSMPVQP

SEQ ID NO: 17 - protein, 418 aa, Streptomyces coelicolor
ADGPAGDSTATTSRGVEIPAFYTPPSELPAADGTLIRHEPLPLALSLPGIDGPLPGRATRLMYKSTDANGEAVAVTGAYVEPAAKWRGDGPRPLVAVAPGTMGQGDQCAASMALEHPLQLNGQTVSVGYEDLSVYRLLLRGVAVVVTDYVGLGTTDRLHTYVNRVDGAHAVLDAVRAARSLDSASVTSDSRVGLFGYSQGGGATAAAAELQPSYAPDVQLAGTYAGAPPADLTEVTKAIDGSDLAGALGWSLNGFLQTEPALRPIADRYINEAGQEALKDLSTMCVGDALFGYGGDSSTDWTRTGQSISDVIRAEPALQSFLAEQRIGSTAPGSPVRVATGVSDDLVPHGQARRLAVDWCGKGGKVTYVPVLIPGVGSGLLNHFAPLLADQGNAIAWLTDRLSGEPAGSNCWSMPVQP

SEQ ID NO: 18 - DNA, 1338 bp, Artificial Sequence
  Synthetic DNA
atgcccccacgtccccgcatgctcgccgcggcgatcaccgccgcgctcgccctcggtgcccaggccgtcccggccgcggcggccgacggcccggccggcgactcgacggccacgacgtcccgcggcgtcgagatccccgccttctacaccccgccgtccgaactccccgcggccgacggcaccctgatccgccatgaaccgctccccctggcgctgagtctgccgggcatcgacggccccctccccggccgggccacccggctgatgtacaagtccaccgacgcgaacggcgaagccgtcgccgtgaccggcgcctacgtcgaaccggccgcgaagtggcgcggcgacgggccccgccccctggtcgccgtcgcgcccggcaccatggggcagggcgaccagtgcgccgcctcgatggccctggagcacccgttgcagctcaacggccagacggtctcggtcggttacgaggacctgtcggtctaccggctgctgctgcgcggcgtcgccgtcgtcgtcaccgactacgtcggcctcggcaccaccgaccggctgcacacctacgtcaaccgcgtcgacggggcccacgccgtgctggacgccgtacgcgccgcacgctcgctcgactcggcgtcggtcacctcggactcccgggtgggtctgttcgggtacagccagggcggcggcgccacggcggcagcggccgagctccagccctcctacgccccggacgtccagctggcgggcacctacgccggggccccgcccgccgacctcaccgaggtgacgaaggcgatcgacggcagcgacctggcgggcgccctgggctggtcgctcaacggcttcctgcagaccgagcccgccctgcggcccatcgccgaccggtacatcaacgaggcggggcaggaggcgctgaaggacctgtcgaccatgtgcgtgggcgacgcgctcttcgggtacggcggcgacagcagcacggactggacgaggaccggccagtccatcagcgacgtcatccgcgccgaacccgcgctgcagagcttcctcgccgagcagcgcatcggctcgaccgcgcccggcagtccggtgcgcgtggccacgggtgtcagtgacgacctggtcccccacggccaggcccgccgactggccgtcgactggtgcggcaagggcgggaaggtgacctacgtgccggtgctgataccgggcgtgggcagcggcctgctcaaccacttcgccccgttgctcgccgaccagggcaacgccatcgcctggctcaccgaccggctctccggcgagcccgccggctccaactgctggagcatgccggtacagccctga

SEQ ID NO: 19 - protein, 419 aa, Corynebacterium jeikeium
MFKKTAALACATAIAFAGFASPAEAAQITKREKVAYSNVFGDLHMRLPKTVDRIEYTTTTSDGKKAQVSGYIVEPVVEWKGEGPRPTVVIGRGTVGQGDQCAPSRNWPLDGQPDPIKAKRAVNLEGIYDWVFASKGVRVVVTDYIGMGTPGMHTYMNRDDQAHAMLDAARAAGVGDGPVAFYGHSQGGGASAAAVEAAGEYAPELNVVGAYASAPPADLEAVQRNIDGSDLMGAIGFTINGLLERYPHLQPLLDKHLNEEGKQALKDLSTMCTGEITDKYGHKKTSEWTKSGKTLDELLKEMPEAREAMEDQRIGLKRPVAPVMVISGRHDKNVEYNQAKELARTWCEKGAQVEYRDDIMPPVGEYNHFIQAVTGGAFGIDFILKRFRGEPLGRTCEAPVGQLQGSVEGSAAMPNVIGS

SEQ ID NO: 20 - protein, 394 aa, Corynebacterium jeikeium
AQITKREKVAYSNVFGDLHMRLPKTVDRIEYTTTTSDGKKAQVSGYIVEPVVEWKGEGPRPTVVIGRGTVGQGDQCAPSRNWPLDGQPDPIKAKRAVNLEGIYDWVFASKGVRVVVTDYIGMGTPGMHTYMNRDDQAHAMLDAARAAGVGDGPVAFYGHSQGGGASAAAVEAAGEYAPELNVVGAYASAPPADLEAVQRNIDGSDLMGAIGFTINGLLERYPHLQPLLDKHLNEEGKQALKDLSTMCTGEITDKYGHKKTSEWTKSGKTLDELLKEMPEAREAMEDQRIGLKRPVAPVMVISGRHDKNVEYNQAKELARTWCEKGAQVEYRDDIMPPVGEYNHFIQAVTGGAFGIDFILKRFRGEPLGRTCEAPVGQLQGSVEGSAAMPNVIGS

SEQ ID NO: 21 - DNA, 1320 bp, Artificial Sequence
  Synthetic DNA
atgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggccgcccagatcaccaagcgcgagaaggtcgcctactccaacgtgttcggcgacctgcacatgcggctcccgaagaccgtggaccggatcgagtacacgaccacgaccagcgacggcaagaaggcccaggtctcgggctacatcgtcgagccggtcgtggagtggaagggcgagggcccccggccgacggtggtcatcggccgcggcacggtcgggcagggcgaccagtgcgccccgagccggaactggcccctcgacggccagcccgacccgatcaaggcgaagcgcgccgtgaacctcgagggcatctacgactgggtgttcgccagcaagggcgtccgcgtcgtggtgaccgactacatcgggatgggcacccccggcatgcacacctacatgaaccgcgacgaccaggcccacgccatgctcgacgcggcgcgggcggccggcgtcggcgacgggcccgtcgcgttctacggccactcccagggcgggggcgcctccgccgcggccgtggaggccgcgggggagtacgcgcccgagctgaacgtcgtgggcgcgtacgcgtccgccccgcccgccgacctggaggccgtccagcgcaacatcgacggctcggacctcatgggcgccatcggcttcaccatcaacggcctgctggagcgctacccgcacctgcagccgctgctcgacaagcacctgaacgaggagggcaagcaggcgctcaaggacctgtccaccatgtgcaccggcgagatcaccgacaagtacggccacaagaagaccagcgagtggaccaagagcggcaagacgctggacgagctgctcaaggagatgcccgaggcgcgggaggcgatggaggaccagcggatcggcctgaagcgcccggtggccccggtgatggtcatctccggccgccacgacaagaacgtcgagtacaaccaggcgaaggagctggcgcgcacctggtgcgagaagggcgcgcaggtggagtaccgcgacgacatcatgccgcccgtcggggagtacaaccacttcatccaggcggtcacgggcggcgccttcggcatcgacttcatcctgaagcggttccgcggggagcccctgggccggacgtgcgaggcgccggtcgggcagctccagggctcggtcgagggctccgccgccatgccgaacgtgatcgggtcg

SEQ ID NO: 22 - protein, 488 aa, Malassezia furfur
MPSMLSLFYLAQSLFLLLLFPLYGHASVLKRGNNDPFYQPPAHWKSKQPGDILRWRKIEPKFIGGDFNVAEAYQLLYRTSQNTPNEPQHTVTTILVPHNAKKDILVVGSVAQDANGQQCTPSAGYTYNSESNFVFWLDETFFLQYLQEGYIMTIPDKEGPKNAFAAGRMEGYMTLDSIRATLNFSKLKLSSNTRIAGYGYSGGAITLGWASSLKPSYAPELNIIGWSFGGTPSNLLGTINHIDGTIFSGLILAGVTGVTDVYPEIHEYIQTVLNSAGREGLEFCRNHCLQEIILRYPLKSIYSYEFQTRGKDVFNNATVQQMFTDLTMGIRPHETPDVPVFMYHAQHDEIVPYDDAHKTAKAWCRNGAQVKFTTYSHYEMGHFTTEITGSVPAFHFIRDLFNGKEVSQGCDFKTEDTLFFNPSVLGGNANEIIDAILGIFGQRIGPEGRILAAKKTVVKGSKSGSSLKSHSHSQTHKHRKDVSTISNA

SEQ ID NO: 23 - protein, 462 aa, Malassezia furfur
SVLKRGNNDPFYQPPAHWKSKQPGDILRWRKIEPKFIGGDFNVAEAYQLLYRTSQNTPNEPQHTVTTILVPHNAKKDILVVGSVAQDANGQQCTPSAGYTYNSESNFVFWLDETFFLQYLQEGYIMTIPDKEGPKNAFAAGRMEGYMTLDSIRATLNFSKLKLSSNTRIAGYGYSGGAITLGWASSLKPSYAPELNIIGWSFGGTPSNLLGTINHIDGTIFSGLILAGVTGVTDVYPEIHEYIQTVLNSAGREGLEFCRNHCLQEIILRYPLKSIYSYEFQTRGKDVFNNATVQQMFTDLTMGIRPHETPDVPVFMYHAQHDEIVPYDDAHKTAKAWCRNGAQVKFTTYSHYEMGHFTTEITGSVPAFHFIRDLFNGKEVSQGCDFKTEDTLFFNPSVLGGNANEIIDAILGIFGQRIGPEGRILAAKKTVVKGSKSGSSLKSHSHSQTHKHRKDVSTISNA

SEQ ID NO: 24 - DNA, 1365 bp, Artificial Sequence
  Synthetic DNA
atgcccagcatgctcagcctcttctacctcgcccagagcctcttcctcctcctcctcttccccctctacggccacgcttccgttcttaagcgcggtaacaacgaccctttctaccagccccccgcccactggaagagcaagcagcccggcgatatccttcgctggcgcaagattgagcctaagttcatcggcggtgactttaacgtcgctgaggcctaccagctcctctaccgcaccagccagaacacccccaacgagccccagcacaccgttactactatcctcgtccctcacaacgctaagaaggacatcctcgtcgtcggtagcgtcgctcaggacgccaacggccagcagtgcactcctagcgctggttacacctacaacagcgagagcaacttcgtcttctggctcgacgagaccttcttcctccagtacctgcaggagggttacattatgactattcctgacaaggagggccctaagaacgccttcgctgctggtcgcatggagggctacatgaccctcgacagcatccgcgccaccctcaacttcagcaagctcaagctcagcagcaacacccgcatcgccggctacggctacagcggcggcgccatcaccctcggctgggccagctccctgaagcctagctacgcccccgagctcaacatcatcggctggagcttcggcggcacccccagcaatctcctgggtaccatcaaccacattgacggtactatcttcagcggtctcatcctcgctggtgtcaccggcgtcaccgacgtctaccccgagatccacgagtacatccagaccgtcctcaacagcgccggccgcgagggcttggagttctgccgcaaccactgcctccaggagatcatcctccgctaccctctcaagagtatctatagttacgagttccagactcgcggtaaggacgtcttcaacaacgccaccgtccagcagatgttcaccgacctcacaatgggcatccgcccccacgagacccccgacgtccccgtcttcatgtaccacgcccagcacgacgagatcgtcccctacgacgacgcccacaagaccgccaaggcctggtgccgcaacggcgcccaggtcaagttcaccacctacagccactacgagatgggccacttcaccaccgagatcaccggcagcgtccccgccttccacttcatccgcgacctcttcaacggcaaggaggtcagccagggctgcgacttcaagaccgaggacaccctcttcttcaaccccagcgtcctcggcggcaacgccaacgagatcatcgacgccatcctcggcatcttcggccagcgcatcggccccgagggccgcatcctcgccgccaagaag

SEQ ID NO: 25 - protein, 449 aa, Aspergillus terreus
MAKEAFILFHFLLLTPLFVWAMPASSNAPLRPTEDAFYSPPSGFESMAPGAILRYRTPPSPIAAFGFARSNLRSAYQVLYRTTDSFGNPIATVTTILIPHNADFNKLHSYQVAQDAADADCSPSFALQQQSTVSGSLALAMPQMEYVFITSALDKGWVVTIPDHLGPSAAFLANTLSGKAVLDNIRAALASGNFTQIEPTARVTMWGYSGGSLATGFAAELQPTYAPELTIAGAALGGTVPKILSVINTVNKGVFTGLVPAGILGLTNEYPAAREIVRDIILPEKAAEFNKAKRLCLTGALREYFGQDIYNYVNSRNAFSSSAARAVLDPNSMGQNTPNIPLFIYKSISDEVSPIADSDELVAKYCEGGANVEYKRDRLSEHASLEVIGAPDAFLWLIDRMAGKPIHAGCTNTTVLTSLDSPRALAVFGASGVNVLLSMLSLPIGPLIP

SEQ ID NO: 26 - protein, 449 aa, Aspergillus terreus; identical to SEQ ID NO: 25

SEQ ID NO: 27 - DNA, 1353 bp, Artificial Sequence
  Synthetic DNA
atggccaaggaggccttcatcctcttccactttctgctcctcacccccctcttcgtctgggccatgcctgccagcagcaacgcccctctgcgccctaccgaggacgccttctacagcccccccagcggcttcgagagcatggcccctggcgccatcctgcgctaccgaacccctcctagccctattgccgccttcggcttcgcccgcagcaacctccgcagcgcctaccaggtcctctaccgcaccaccgacagcttcggcaaccccattgccaccgtcaccaccatcctcatcccccacaacgccgacttcaacaagctccacagctaccaggtcgcccaggacgccgccgacgctgactgctctcccagcttcgccctccagcagcagagcaccgtcagcggcagcctcgccctggccatgccccagatggagtacgtctttatcaccagcgccctcgacaagggctgggtcgtcaccatccccgaccacctcggccctagcgccgcctttctcgccaacaccctcagcggcaaggccgtcctcgacaacatccgcgctgccctcgcctccggcaacttcacccagatcgagcccaccgcccgcgtcaccatgtggggttacagcggtggctctctggccacgggctttgccgccgagctgcagcctacctacgcccctgagctgacgattgctggcgctgccctgggcggcactgtccccaagatcctcagcgtcatcaacaccgtcaacaagggcgtctttaccggcctcgtccccgccggcatcctcggcctcaccaacgagtaccccgccgcccgcgagatcgtccgcgacatcatcctccccgagaaggccgccgagtttaacaaggccaagcgcctctgcctcaccggcgctctccgagagtactttggccaggacatctacaactacgtcaacagccgcaacgccttcagcagctctgccgctcgagccgtcctggaccccaacagcatgggccagaacacccccaacatccccctgttcatctacaagagcatcagcgacgaggtctcgcctattgccgacagcgacgagctggtcgccaagtactgcgagggtggcgctaacgtcgagtacaagcgcgaccgcctcagcgagcacgccagcctcgaggtcatcggcgcccctgatgcctttctctggctcatcgaccgcatggccggcaagcccatccacgccggctgcaccaacaccaccgtcctcaccagcctcgacagccctcgggctctggctgtctttggtgcctctggcgtgaacgtcctcctcagcatgctcagcctccccatcggccccctcattccctaatag

SEQ ID NO: 28 - protein, 453 aa, Aspergillus oryzae
MARLSLLLAPLFAVLPLVSAFPAQLPARATAPALPVDDPFYIPPEGFESSAPGTILRHRTPPNPIAALGFAKVNIQAAHQILYRTSDSSGNAIATVSTILIPHNADYSKLLSYQVAEDAADPNCAPSYALQLEAAHDGILGLVIPQVELLFFGAALNKGWVVTVPDHLGPKAAFLANNLSGQAVLDNIRAALASSSFTNITSDPTIALWGYSGGSLASGFAAELHPTYAPELNIVGAALGGTVPKIRPVIDAVNKGLFVGLVPSGIQGLANEYPDIQQLINDGLKPSKRADFNKTQNPCLSGDILQYLGQDIYDYTNDRNIFDQPAAVKVMDANAMGQHVPKIPLLVYKSVGDEISPVNDTDALVETYCNAGASVEYKRDELSEHASLEITGSADGLLWIMDRMNNKPVQQGCTKSTAVTGLADPKALLALGDEILTLLKAILAGPIGPGVVG

SEQ ID NO: 29 - protein, 453 aa, Aspergillus oryzae
MARLSLLLAPLFAVLPLVSAFPAQLPARATAPALPVDDPFYIPPEGFESSAPGTILRHRTPPNPIAALGFAKVNIQAAHQILYRTSDSSGNAIATVSTILIPHNADYSKLLSYQVAEDAADPNCAPSYALQLEAAHDGILGLVIPQVELLFFGAALNKGWVVTVPDHLGPKAAFLANNLSGQAVLDNIRAALASSSFTNITSDPTIALWGYSGGSLASGFAAELHPTYAPELNIVGAALGGTVPKIRPVIDAVNKGLFVGLVPSGIQGLANEYPDIQQLINDGLKPSKRADFNKTQNLCLSGDILQYLGQDIYDYTNDRNIFDQPAAVKVMDANAMGQHVPKIPLLVYKSVGDEISPVNDTDALVETYCNAGASVEYKRDELSEHASLEITGSADGLLWIMDRMNNKPVQQGCTKSTAVTGLADPKALLALGDEILTLLKAILAGPIGPGVVG

SEQ ID NO: 30 - DNA, 1365 bp, Artificial Sequence
  Synthetic DNA
atggcccgcctcagcctcctcctcgcccccctcttcgccgtcctgcccctcgtcagcgcctttcctgcccagctccctgcccgcgccactgctcctgccctccctgtcgacgaccccttctacatcccccccgagggcttcgagagcagcgcccctggcaccatcctccgccaccgcacccctcccaaccccattgccgccctcggcttcgccaaggtcaacatccaggccgcccaccagatcctctaccgcaccagcgacagcagcggcaacgccattgccacggtctctaccattctcatcccccacaacgccgactacagcaagctcctctcgtaccaggtcgccgaggacgccgccgaccccaactgcgcccccagctacgccctccagctcgaggctgcccacgacggcatcctcggcctcgtcatcccccaggtcgagctgctgttcttcggcgccgccctcaacaagggctgggtcgtcaccgtccccgaccacctcggccccaaggccgccttcctcgccaacaacctcagcggccaggccgtcctcgacaacatccgcgccgctctcgccagcagcagcttcaccaacatcaccagcgaccccacgatcgccctctggggctactctggcggctctctcgcctctggcttcgccgccgagctgcaccccacctacgcccccgagctgaacattgtgggcgctgctctgggcggcactgtccctaagatccgccccgtcatcgatgccgtgaacaagggcctcttcgtcggcctcgtccccagcggcatccagggcctcgccaacgagtaccccgacatccagcagctcatcaacgacggcctcaagcccagcaagcgcgccgacttcaacaagacccagaacctctgcctcagcggcgacatcctccagtacctcggccaggacatctacgactacaccaacgaccgcaacatcttcgaccagcctgccgccgtcaaggtcatggacgccaacgccatgggccagcacgtgcctaagattcctctgctcgtctacaagagcgtcggcgacgagatctcccccgtcaacgacaccgacgccctcgtcgagacctactgcaacgctggcgcctctgtcgagtacaagcgcgacgagctgagcgagcacgccagcctcgagatcaccggcagcgctgatggcctcctctggatcatggaccgcatgaacaacaagcccgtccagcagggctgcaccaagagcaccgccgtcactggcctggccgatcctaaggccctgctcgctctgggcgatgagattctcactctcctcaaggccatcctggccggccccatcggccctggcgtcgtcggctaatag

SEQ ID NO: 31 - protein, 469 aa, Aspergillus fumigatus
MQHGRSEEQSSQKFGVSHSASKMHWAIQLWMVSFSLSVFALPTGRDALVRPLEDPFYSAPKGFESTVPGTILRWRNPPNPISAFGFAPINLAASYQLLYRSTDSFGEPIAAASTILVPHNADNTKLLSFQAAEDAANPNCAPSYAFQLDSATDDELGLIMPQAELVLIIAALDKGWVVTVPDHLGPNATFLANNLSGHVVLDNIRAALRSSAFSGISPKATITLWGYSGGSLASGLAAELRASYAPELNIAGAALGGTVPKIMPVFNTVNKGIYAGLLPAGMQGLSNEYPAIEKILYDHLVPAKKADFVKTKNLCIVEDLLTNSFQDFYRYITDANMLKDPEVTRVLGENAMGQHVPDIPLFVYKSTNDDVSPVGDTDALVSGYCAAGGKVEYYRDELSNHATMAVIGVPNALLWLKDRMNGVPARAGCKTQTALTGLLDPRTLAVLGIDLIKVLLALLSAPVGTFVIG

SEQ ID NO: 32 - protein, 469 aa, Aspergillus fumigatus; identical to SEQ ID NO: 31

SEQ ID NO: 33 - DNA, 1413 bp, Artificial Sequence
  Synthetic DNA
atgcagcacggccgcagcgaggagcagagcagccagaagttcggcgtcagccacagcgccagcaagatgcactgggctattcagctctggatggtcagcttcagcctcagcgtctttgccctccctaccggccgagatgccctcgtccgacctctcgaggaccccttttacagcgcccctaagggcttcgagagcaccgtccccggcaccatcctccgctggcgcaacccccccaaccccatttccgcctttggcttcgcccctatcaacctcgctgcctcctaccagctcctctaccgcagcaccgacagcttcggcgagcccattgccgctgcttctacgattctggtcccccacaacgccgacaacaccaagctcctcagcttccaggccgccgaggacgccgctaaccctaactgcgcccccagctacgccttccagctcgacagcgccaccgacgacgagctgggcctgatcatgcctcaggctgagctggtcctcattatcgccgccctcgacaagggctgggtcgtcacggtccctgaccacctgggccccaacgccaccttcctcgccaacaacctcagcggccacgtcgtcctcgacaacatccgagctgccctccgctcctccgcctttagcggcatcagccccaaggccaccatcaccctctggggctacagcggcggctctctcgcctctggcctggccgctgagctgcgagctagctacgcccctgagctgaacattgccggcgctgctctgggcggcactgtccctaagatcatgcccgtctttaacaccgtcaacaagggcatctacgctggcctgctgcctgccggtatgcagggtctcagcaacgagtaccccgccatcgagaagatcctctacgaccacctggtccccgccaagaaggccgacttcgtcaagaccaagaacctctgcatcgtcgaggacctgctcaccaacagcttccaggacttctaccgctacatcaccgacgccaacatgctcaaggaccccgaggtcacccgcgtcctcggcgagaacgccatgggccagcacgtccccgacatccccctgttcgtctacaagagcaccaacgacgacgtcagccccgtcggcgacacggatgccctggtcagcggctactgtgctgctggcggtaaggtggagtactaccgcgacgagctgagcaaccacgccaccatggctgtgatcggcgtccccaacgccctcctgtggctgaaggatcgcatgaacggcgtccctgcccgagccggctgcaagacccagaccgccctcacgggcctgctggaccctcgcaccctcgccgtcctcggcatcgacctcatcaaggtcctgctcgccctcctcagcgcccctgtcggcaccttcgtcatcggctaatag

SEQ ID NO: 34 - protein, 418 aa, Aspergillus niger
MYIPSVLLLAASLFHGATALPTPGSTPIPPSQDPWYSAPEGFEEADPGAILRVRPAPGNLTVVVGNASAAYNILYRTTDSQYKPSWAVTTLLVPPVAASAAVNQSVLLSHQIAYDSFDVNASPSYAMYTSPPSDIILALQRGWFVNVPDYEGPNASFTAGVQSGHATLDSVRSVLASGFGLNEDAQYALWGYSGGALASEWAAELQMQYAPELNIAGLAVGGLTPNVTSVMDTVTSTISAGLIPAAALGLSSQHPETYEFILSQLKTTGPYNRTGFLAAKDLTLSEAEVFYAFQNIFDYFVNGSATFQAEVVQKALNQDGYMGYHGFPQMPVLAYKAIHDEISPIQDTDRVIKRYCGLGLNILYERNTIGGHSAEQVNGNARAWNWLTSIFDGTYAQQYKTEGCTIRNVTLNTTSSVY

SEQ ID NO: 35 - protein, 418 aa, Aspergillus niger; identical to SEQ ID NO: 34

SEQ ID NO: 36 - DNA, 1260 bp, Artificial Sequence
  Synthetic DNA
atgtacatccccagcgtcctgctcctcgccgccagcctcttccacggcgccaccgccctccctacccctggcagcactcccattcctcccagccaggacccctggtacagcgcccctgagggcttcgaggaggccgaccctggcgccattctccgcgtccgacctgctcctggcaacctcaccgtcgtcgtcggcaacgccagcgccgcctacaacatcctctaccgcaccaccgacagccagtacaagccctcttgggccgtcaccaccctgctcgtcccccctgtcgccgcctctgccgccgtcaaccagagcgtcctcctcagccaccagatcgcctacgacagcttcgacgtgaacgcttctcccagctacgccatgtacaccagcccccccagcgacatcatcctcgccctccagcgcggctggttcgtcaacgtccccgactacgagggccccaacgccagcttcactgccggcgtccagagcggccacgccaccctcgactctgtccgcagcgtcctggcctctggcttcggcctcaacgaggacgcccagtacgccctctggggctactctggcggcgctctcgcctctgagtgggctgccgagctgcagatgcagtacgcccccgagctgaacattgccggcctcgccgtcggcggcctcacccccaacgtcaccagcgtcatggacaccgtcaccagcaccatctctgccggcctcatccctgccgctgccctcggcctcagcagccagcaccccgagacctacgagttcatcctcagccagctcaagaccaccggcccctacaaccgcaccggctttctcgccgccaaggatctcactctcagcgaggccgaggtgttttacgccttccagaacatcttcgactacttcgtcaacggctctgctaccttccaggctgaggtggtccagaaggccctcaaccaggacggctacatgggctaccacggcttcccccagatgcccgtcctcgcctacaaggccatccacgacgagatcagccccatccaggacaccgaccgcgtcatcaagcgctactgcggcctcggcctgaacatcctgtacgagcgcaacaccatcggcggccactctgccgagcaggtcaacggcaacgcccgagcctggaactggctcaccagcatcttcgacggcacctacgcccagcagtacaagaccgagggctgcaccatccgcaacgtcaccctcaacaccacctccagcgtctactaatag

SEQ ID NO: 37 - protein, 445 aa, Aspergillus clavatus
MYSTTLTVGSFLALTLTALSASLPQIPQQQTGSPAAALPPSKDAWYTAPDNLTFSTPGTVLRVRRDPLNLYALIGTNISCASYNILYRTTNVQNRPTFAVTTLFVPASSQAASDRLLSYQIPYNSPWVDASPSYALATDLNGTFGDIAAALEQGWHVSVPDFEGPQATFAVGVNEGYAVLDSVRAATDFSSSLAGSPKRWQAALWGYSGGSIASVFAAELHAAYAPELTIAGVAVGGLPALFAKVLDAVNRSPAAGVIPLALWGYANAYPAVERYLLNRLTPEARASDVFTKARNMTGLEAGVAYLGADVYSYFVGGRDDIQSSPLLHFLFGTQGTMGRHGLPHMPVYVYHSINDKLALVNYVDALIEHYCAEGSLERGKGGVRIVYERNSVGGHGAEAINQDADALKFLSQALDGQLGAGWPNRQGEGCVIRNVSVGTDTSLDW

SEQ ID NO: 38 - protein, 445 aa, Aspergillus clavatus; identical to SEQ ID NO: 37

SEQ ID NO: 39 - DNA, 1341 bp, Artificial Sequence
  Synthetic DNA
atgtacagcaccaccctcaccgtcggctcgttcctcgccctcaccctcaccgccctcagcgccagcctcccccagatccctcagcagcagaccggctctcctgccgctgccctgccgccttctaaggacgcctggtacaccgcccctgacaacctcaccttcagcacccctggcaccgtcctccgagtccgccgcgaccctctgaacctgtacgctctgattggcaccaacatcagctgcgccagctacaacatcctctaccgcaccaccaacgtccagaaccgccccaccttcgccgtcaccaccctcttcgtccccgccagcagccaggccgccagcgaccgcctcctcagctaccagatcccctacaacagcccctgggtcgacgcctctccttcttacgctctcgccactgacctcaacggcaccttcggcgacattgccgctgccctcgagcagggctggcacgtcagcgtccccgacttcgagggcccccaggccacgtttgctgtgggcgtcaacgagggctacgccgtcctcgacagcgtccgagccgccaccgacttcagcagcagcctcgccggctctcctaagcgctggcaggctgccctctggggctacagcggcggctcgatcgccagcgtctttgccgccgagctgcacgccgcctacgcccctgagctgacgattgccggcgtcgctgtgggcggcctccccgccctcttcgccaaggtcctcgacgccgtcaaccgctctcccgccgctggcgtcattcccctggccctgtggggctacgccaacgcctaccccgccgtcgagcgctacctcctcaaccgcctcacccccgaggcccgagccagcgacgtctttacgaaggctcgcaacatgacgggccttgaggctggcgtggcctacctcggcgctgacgtctacagctacttcgttggcggccgagatgacatccagagcagccccctgctccacttcctgttcggcacccagggcaccatgggccgccacggcctcccccacatgcccgtctacgtctaccacagcatcaacgacaagctcgccctcgtcaactacgtcgacgccctgatcgagcactactgcgccgagggctctctcgagcgaggcaagggcggcgtccgcatcgtctacgagcgcaacagcgtcggcggccacggcgctgaggccatcaaccaggacgccgacgccctcaagttcctgtctcaggctctcgacggccagctgggtgctggctggcctaaccgccagggcgagggctgcgtcatccgcaacgtcagcgtcggcaccgacaccagcctcgactggtaatag

SEQ ID NO: 40 - protein, 447 aa, Aspergillus oryzae
MFGLGSLLSFFFVFATCLAHPGSLANRAMVTPPSADPFYKPPSGYENTEPGTILRHRRVTNPIALIDPIKLKLAAAYQILYRSTDNWANATATVTTVLVPENANTSRLLSYQIPEDSSSVDCAPSYILQTGIDDSDILSKTGIQSHTVLFRAALEKGWIVSLPDHEGPKAAFLANRRAGHAVLDGIRAVLGSSGFTTVSPNASVAMWGYSGGSLATGFAAELQTTYAPDLDMIVGAALGGLIGDIEVVTYTVNKGPFVGLNFGGINGIAHEYPDIAALFEEQLVESKKAKFYDANHNCFLANIIKYPFQDLFSYFKDPSILSYPQMQQALKDNNLGQNPPKMPIFVYKGALDEISPVSSTEVVVSQYCAGGTVVNYKNVLTHEHILLQLDGFLEAFTWLEKRMDGEAMETDCSRTDELLPISEPGSLGVVVPTVEGVVSDVVGTVLG

SEQ ID NO: 41 - protein, 447 aa, Aspergillus oryzae; identical to SEQ ID NO: 40

SEQ ID NO: 42 - DNA, 1347 bp, Artificial Sequence
  Synthetic DNA
atgttcggcctcggcagcctcctcagcttcttcttcgtctttgccacctgcctcgcccaccctggcagcctcgccaaccgagccatggtcacccctcccagcgccgaccccttttacaagccccccagcggctacgagaacaccgagcccggcaccattctccgacaccgccgcgtcaccaaccccattgccctcatcgaccccatcaagctcaagctcgccgccgcctaccagattctctaccgcagcaccgacaactgggccaacgccaccgccaccgtcaccaccgtcctcgtccccgagaacgccaacaccagccgcctgctcagctaccagatccccgaggacagcagctctgtcgactgcgcccccagctacatcctccagaccggcatcgacgacagcgacatcctgagcaagaccggcatccagagccacaccgtcctgtttcgagccgccctcgagaagggctggatcgtcagcctccctgaccacgagggccctaaggccgcctttctggccaaccgacgagccggccacgccgtcctcgacggcattcgagctgtcctgggcagcagcggctttaccaccgtcagccccaacgcctctgtcgccatgtggggctactctggcggctctctcgccaccggctttgccgccgagctgcagaccacctacgcccccgacctcgacatgattgtcggcgctgccctcggcggcctgattggcgacatcgaggtcgtcacctacactgtcaacaagggcccttttgtgggcctcaacttcggcggcatcaacggcattgcccacgagtaccccgacattgccgccctgtttgaggagcagctggtcgagtctaagaaggccaagttctacgacgccaaccacaactgctttctcgccaacatcatcaagtaccccttccaggacctcttcagctacttcaaggaccccagcatcctcagctacccccagatgcagcaggccctcaaggacaacaacctcggccagaacccccccaagatgcccatcttcgtctacaagggcgctctcgacgagattagccccgtcagcagcaccgaggtcgtggtctctcagtattgcgctggcggcactgtcgtcaactacaagaacgtcctcacccacgagcacatcctgctccagctcgacggcttcctcgaggccttcacctggctcgagaagcgaatggacggcgaggccatggagaccgactgcagccgaactgatgagctgctccctattagcgagcctggctctctcggtgtcgtcgtccctaccgtcgagggcgtcgtgtctgacgtcgtcggcaccgtcctcggctaatag

SEQ ID NO: 43 - protein, 448 aa, Fusarium verticillioides
MFPRFILLSVAAFSTLGLSVPVQERQQPILPTEDSFYKVPDNINDYPPGTIIDYRKPPSPIAAFGVSPVNLKDTWQISYRTNDNFGKALSTVLTVLVPYKADFTKVLSYQVAEDAAYAGCAPSYALQFASATGGPLGTIVTQAELLLMEAALEQGWVVVAPDHEGPDAAYLANKLAGYATLDGIRAAINSANFTGISKNPTVGMWGYSGGSIASAAAAELQQSYAPELRIAGAALGGTVPNITSVIKSINKGPLAGIIPPGMIGLTKQYPILKLGLDAAVKPEYKAKFDAVGKQCFVADILSFLGQDVVGMLKDPAILTEEPAKGILDENALGKHNPSIPLFIYKSIQDEVSPIKDTDDLVKFYCSGGTKVQYDRDLLSEHGSLAIIGAGKALAWLKDIMNGRKGPSQCKTSNVVSSLLDLRGSITLSAALIEALVDLVGKPVGPIIG

SEQ ID NO: 44 - protein, 448 aa, Fusarium verticillioides; identical to SEQ ID NO: 43

SEQ ID NO: 45 - DNA, 1350 bp, Artificial Sequence
  Synthetic DNA
atgttcccccgcttcatcctcctcagcgtcgccgccttcagcaccctcggcctcagcgtccccgtccaggagcgccagcagcccatcctccccaccgaggacagcttctacaaggtccccgacaacatcaacgactacccccctggcaccatcatcgactaccgcaagccccccagccccattgccgccttcggcgtcagccccgtcaacctcaaggacacctggcagatcagctaccgcaccaacgacaacttcggcaaggccctcagcaccgtcctgaccgtcctcgtcccctacaaggccgacttcaccaaggtgctgtcttaccaggtcgctgaggacgccgcctacgccggctgcgcccctagctacgccctccagttcgcctctgccactggcggccctctcggcacgatcgtcactcaggccgagctgctcctcatggaggccgccctcgagcagggctgggtcgtcgtcgcccctgaccacgagggccctgatgccgcttacctcgccaacaagctcgctggctatgccaccctcgacggcattcgagctgctatcaacagcgccaacttcaccggcatcagcaagaaccccaccgtcggcatgtggggctactctggcggctcgattgcttctgctgccgccgctgagctgcaacagtcttacgcccctgagctgcgaattgctggcgctgccctgggcggcactgtccccaacatcaccagcgtcatcaagagcatcaacaagggccccctcgccggcatcatcccccctggcatgatcggcctcaccaagcagtaccccatcctcaagctcggcctcgacgccgccgtcaagcccgagtacaaggccaagttcgacgccgtcggcaagcagtgcttcgtcgccgacatcctctcgttcctcggccaggacgtcgtcggcatgctcaaggaccccgccatcctcaccgaggagcccgctaagggtatcctcgatgagaacgctctgggcaagcacaaccccagcatccccctgttcatctacaagagcatccaggacgaggtcagccccatcaaggacaccgacgacctcgtcaagttctactgctctggcggtactaaggtccagtacgaccgcgacctcctcagcgagcacggcagcctcgccatcattggcgctggcaaggccctggcctggctcaaggacatcatgaacggccgaaagggccctagccagtgcaagaccagcaacgtcgtcagcagcctcctcgacctccgcggcagcatcaccctgtctgccgccctcatcgaggccctcgtcgacctcgtcggcaagcccgtcggccccatcatcggctaatag

SEQ ID NO: 46 - protein, 445 aa, Fusarium graminearum
MALDRLLFLLGFWLGLVGAAQAALSSEPLPPSKDPWYTAPPGFENTEPGTVLRVRPAPGNLTSVTSNCSASYNILYRTTDSHFKPAWAVTTLLIPELGPESLAHQKYQQSALMSIQVAYDSPDVDASPSNTMYTASNFSSIYYEAALGQGLFVSVPDYEGPLAAFSAGVISGYATLDSIRAVLSLGLGLNMTNTPSVALWGYSGGAFATEWASELAVQYAPELITGPVIGAALGAPLANITSLLYDVNGKPGAGLVPNMLLGLTSQYPDVRKYLISKLNDDGQYNKTGFLAAEGFTINEAGVAFYGIDINKYFQKGTDILSDPKIVALLNQEGLLGYNGTPRWPLFIYQAIHDEVTPIADTDAVVNRYCAVGADIHFERNTIGGHYQEADNSYEAAFQWLLDIYSGQRDTKGCVIKEVTRNITGSVLQTRENVQKSGVDFWRSAW

SEQ ID NO: 47 - protein, 445 aa, Fusarium graminearum; identical to SEQ ID NO: 46

SEQ ID NO: 48 - DNA, 1341 bp, Artificial Sequence
  Synthetic DNA
atggccctcgaccgcctcctcttcctcctcggcttttggctgggcctggtgggcgctgctcaggctgccctctcgtctgagcccctgcctccgtctaaggacccttggtacaccgcccctcccggctttgagaacaccgagcccggcaccgtcctccgagtccgacctgcccctggcaacctcaccagcgtcaccagcaactgcagcgccagctacaacatcctctaccgcaccaccgacagccactttaagcccgcctgggccgtcaccaccctgctcattcccgagctgggccctgagtctctggctcatcagaagtaccagcagagcgccctcatgagcatccaggtcgcctacgactctcctgacgtcgacgccagccccagcaacaccatgtacaccgcttctaacttttcgtctatctactacgaggccgctctgggccagggtctctttgtcagcgtccccgactacgaaggtcctctcgccgccttttctgccggcgtcatcagcggctacgccaccctcgacagcattcgcgccgtcctgtctctcggcctcggcctcaacatgaccaacaccccctctgtcgccctctggggctactctggcggcgcctttgctactgagtgggcttctgagctggccgtccagtacgcccctgagctgatcaccggccctgtcattggcgctgccctgggcgcccctctcgccaacatcaccagcctgctctacgacgtcaacggcaagcctggcgctggccttgtccctaacatgctcctcggcctcaccagccagtaccccgacgtccgcaagtacctcatcagcaagctcaacgacgacggccagtacaacaagaccggcttcctcgccgccgagggcttcaccattaacgaggccggcgtcgccttctacggcatcgacatcaacaagtacttccagaagggcaccgacatcctcagcgaccccaagatcgtcgccctcctcaaccaggagggcctcctcggctacaacggcaccccccgctggcccctgttcatctaccaggccatccacgacgaggtcacccccattgccgacaccgacgccgtcgtcaaccgatactgcgccgtcggtgccgatatccacttcgagcgcaacaccatcggcggccactaccaggaggccgacaactcgtacgaggccgccttccagtggctcctcgacatctacagcggccagcgcgacaccaagggctgcgtcatcaaggaggtcacccgcaacattactggctcggtgctccagacccgcgagaacgtccagaagtccggcgtcgacttttggcgatctgcttggtaatag

SEQ ID NO: 49 - protein, 458 aa, Kurtzmanomyces sp.
MRFFLRAVLGLAVTATAALAAPLEPRAALPDPNEDPFYSTPSNIETFANGQIIQSRKVPTDIGNSNNAASYQLSYRTTNTQEDAVANVATIWIPAKPSSPPRIFTYQVYEDSTQLDCAPSYSYLTGYDQPNKATAVLDTPIVISWALQQGYYVVSADHEGARSAFIAGYEEGMAALDGIRALRNYAKLPQDSAVGAYGYSGGAHATVWATSLAAAYAPEINFIGAAHGGTPVSAKDTFTFINGGFFAGFAIAGVSGLANAHPDMEAFIQPRLNAEGVKTLKQIRSRGFCLPEVVTTYPFKNVFALVNDTNLLTEQPISGILQQETLVQSEASYAVPVPKFPRFLWHAALDEIVPYVPVTEYVKEQCAKGANINFNTYPIAEHLTAEIFGLVPGLWFLSQAYEGKAPAVQCGTALPAAPSAQQVLGNDLANQLSSLNGKQSPFGKPFGPISPTSLDKLL

SEQ ID NO: 50 - protein, 439 aa, Kurtzmanomyces sp.
AAPLEPRAALPDPNEDPFYSTPSNIETFANGQIIQSRKVPTDIGNSNNAASYQLSYRTTNTQEDAVANVATIWIPAKPSSPPRIFTYQVYEDSTQLDCAPSYSYLTGYDQPNKATAVLDTPIVISWALQQGYYVVSADHEGARSAFIAGYEEGMAALDGIRALRNYAKLPQDSAVGAYGYSGGAHATVWATSLAAAYAPEINFIGAAHGGTPVSAKDTFTFINGGFFAGFAIAGVSGLANAHPDMEAFIQPRLNAEGVKTLKQIRSRGFCLPEVVTTYPFKNVFALVNDTNLLTEQPISGILQQETLVQSEASYAVPVPKFPRFLWHAALDEIVPYVPVTEYVKEQCAKGANINFNTYPIAEHLTAEIFGLVPGLWFLSQAYEGKAPAVQCGTALPAAPSAQQVLGNDLANQLSSLNGKQSPFGKPFGPISPTSLDKLL

SEQ ID NO: 51 - DNA, 1371 bp, Artificial Sequence
  Synthetic DNA
atgtaccgcaagctcgccgtcatcagcgcctttctcgccaccgcccgagctgcccctctcgagcctcgagctgccctgcctgaccccaacgaggaccccttctacagcacccccagcaacatcgagacgttcgccaacggccagatcatccagagccgcaaggtccccaccgacatcggcaacagcaacaacgccgccagctaccagctcagctaccgcaccaccaacacccaggaggacgccgtcgccaacgtcgccaccatctggatccccgccaagcccagcagccctccccgcatcttcacctaccaggtctacgaggacagcacccagctcgactgcgcccccagctactcctacctcaccggctacgaccagcccaacaaggccaccgccgtcctcgacacccccatcgtcatcagctgggccctccagcagggctactacgtcgtcagcgccgaccacgagggcgcccgctccgcctttatcgccggctacgaggagggcatggccgccctggacggcatccgcgccctgcgcaactacgccaagctcccccaggactccgccgtcggcgcttacggctactctggcggcgcccacgccactgtctgggccacttccctcgccgctgcctacgcccccgagatcaacttcatcggcgctgcccacggcggcactcccgtcagcgccaaggacaccttcacctttattaacggcggcttcttcgccggcttcgccattgccggcgtctctggcctggccaacgcccaccccgacatggaggccttcatccagccccgcctcaacgccgagggcgtcaagaccctcaagcagatccgctcccgcggcttctgcctccccgaggtcgtcaccacctaccccttcaagaacgtctttgccctcgtcaacgacaccaacctgctcaccgagcagcccatcagcggcatcctccagcaggagacgctcgtccagagcgaggcctcctacgccgtccctgtccccaagttcccccgcttcctctggcacgccgccctggacgagatcgtcccctacgtccccgtcacggagtacgtcaaggagcagtgcgccaagggcgccaacatcaacttcaacacctaccccattgccgagcacctcaccgccgagattttcggcctcgtccccggcctctggttcctcagccaggcctacgagggcaaggcccctgctgtccagtgcggcactgccctccctgccgccccttctgcccagcaggtcctcggcaacgacctcgccaaccagctcagcagcctcaacggcaagcagagccccttcggcaagcccttcggccccatcagccccacctccctggacaagctcctctaatag

SEQ ID NO: 52 - protein, 484 aa, Debaryomyces hansenii
MNFKSLFFICLILACLESIIAGPIKVLTPSEDDFYSLPDGYEDKEPGTILKIRKPPQRLRGIYFPILVKGAWQALVRTTDSHGNATAIVTTIIEPFNADPSKVVSYQVMQDSASINCSPSYSFLYGASMNTVFAQLEMYVIDVALMKGWYVVIPDYEGPKASFTAGKQSGQSTLDSIRAVLKTTNVTGIKEDAKVAMWGYSGGTVATGWAAALQPTYAKELKSNLIGAAMGGFVTNITSTAEGVDGTIFAGLTASAIAGLTNEYSQLKSMVQKLLMPSKLVAYNKAQTLCFLPSIVTFAFQKFFTGPNRYFDEGWALFNDPTIKKVIAENTLALKKGDPVPEIPLFVFHGELDKIVAYKDAVRTYNNWCEWGIDSYEFAIDETTGHITEFIEGTPAAIAWLTKMFNGEKPVKGCKSTKRFTNLLYPGANTSVANVLSDSVKTVLGENFGPNAENIDKGSRNATHVKRSLEELGVSLDFPPEGSM

SEQ ID NO: 53 - protein, 464 aa, Debaryomyces hansenii
AGPIKVLTPSEDDFYSLPDGYEDKEPGTILKIRKPPQRLRGIYFPILVKGAWQALVRTTDSHGNATAIVTTIIEPFNADPSKVVSYQVMQDSASINCSPSYSFLYGASMNTVFAQLEMYVIDVALMKGWYVVIPDYEGPKASFTAGKQSGQSTLDSIRAVLKTTNVTGIKEDAKVAMWGYSGGTVATGWAAALQPTYAKELKSNLIGAAMGGFVTNITSTAEGVDGTIFAGLTASAIAGLTNEYSQLKSMVQKLLMPSKLVAYNKAQTLCFLPSIVTFAFQKFFTGPNRYFDEGWALFNDPTIKKVIAENTLALKKGDPVPEIPLFVFHGELDKIVAYKDAVRTYNNWCEWGIDSYEFAIDETTGHITEFIEGTPAAIAWLTKMFNGEKPVKGCKSTKRFTNLLYPGANTSVANVLSDSVKTVLGENFGPNAENIDKGSRNATHVKRSLEELGVSLDFPPEGSM

SEQ ID NO: 54 - DNA, 1446 bp, Artificial Sequence
  Synthetic DNA
atgtaccgcaagctcgccgtcatcagcgcctttctcgccaccgcccgagccggccccattaaggtcctcacccccagcgaggacgacttctacagcctccccgacggctacgaggacaaggagcccggcaccatcctcaagatccgcaagcccccccagcgcctccgcggcatctacttccccatcctcgtcaagggcgcctggcaggccctcgtccgcaccaccgacagccacggcaacgccacggccatcgtcaccaccatcatcgagcccttcaacgccgacccctccaaggtcgtcagctaccaggtcatgcaggacagcgccagcatcaactgcagccccagctacagcttcctctacggcgccagcatgaacaccgtctttgcccagctcgagatgtacgtcatcgacgtcgccctgatgaagggctggtacgtcgtcatccccgactacgagggccccaaggccagcttcaccgccggcaagcagagcggccagagcaccctcgacagcatccgcgccgtcctcaagaccaccaacgtcaccggcatcaaggaggacgccaaggtcgccatgtggggctacagcggcggcaccgtcgctactggctgggctgccgccctccagcctacttacgccaaggagctgaagtccaacctcattggcgccgccatgggcggcttcgtcaccaacatcaccagcaccgccgagggcgtcgacggcaccatcttcgccggcctcaccgcctcggccattgccggcctgacgaacgagtacagccagctcaagagcatggtccagaagctcctcatgcccagcaagctcgtcgcctacaacaaggcccagacgctctgcttcctcccctccatcgtcaccttcgccttccagaagttcttcacgggccccaaccgctacttcgacgagggctgggccctcttcaacgaccccaccatcaagaaggtcattgccgagaacaccctcgccctgaagaagggcgaccccgtccccgagatccccctcttcgtctttcacggcgagctggacaagattgtcgcctacaaggacgccgtccgcacctacaacaactggtgcgagtggggcatcgacagctacgagttcgccatcgacgagactaccggccacatcaccgagttcatcgagggcacgcctgccgccattgcctggctcaccaagatgttcaacggcgagaagcccgtcaagggctgcaagagcaccaagcgcttcaccaacctgctctaccctggcgccaacaccagcgtcgccaacgtcctcagcgacagcgtcaagaccgtcctcggcgagaacttcggccccaacgccgagaacatcgacaagggcagccgcaacgccacccacgtcaagcgcagcctggaggagctgggcgtcagcctcgacttcccgccggagggctccatgtaatag

SEQ ID NO: 55 - DNA, 1440 bp, Trichoderma reesei
atgtaccgcaagctcgccgtcatcagcgcctttctggccacggcccgagccgcccctcagaaggtcctcaagcccacggaggacagcttctacgacgcccccaagggcttcgaggacgccgagatcggcaccatcctcaagatccgcaagaccccccacatgctccgcagcgtctacatccccatcaacgtccagaacagctggcagatcctcgtccgcagcgagtcggccgagggcaacgccacggccatcgtcaccaccgtcatcgagccctacaacgccgaccccagcaagctcgtcagctaccaggtcgccgaggacagcagcagcgagaactgcgccgtcagctacagcctcgagttcggcgccagcatggacaccatcattgcccaggtcgagatgtacttcatgcaggccgccctcgagcagggctactacgtcgtcacccctgactacgagggccctcaggccagctttactgccggccgacaggctggccacgccgtcctcgacagcattcgcgccaccctcgccagcagcaacgtcaccggcgtcgaccccgacgccgaggtcgtcatgtggggctactctggcggctctctcgcctctggctgggccgctgccctgcagcctaagtacgcccccgagctggagggccagattctcggcgctgccctgggcggcttcgtcaccaacatcaccctcaccgccgtcagcgtcgacgacaacatcttcgccggcctgatcgccagcgccatcaacggcctcatgaacgagtaccccgagttcagcgagctggccaaggacatgatccgccccgagcgcctcgagaacttcctcaaggccgacagctactgcatggtccccagcctcatccactacgccttcgacaacttctttgtcggcaacgacagctacttcacccagggcctcgacgtctttaagatccccttcgtccaggacatgatcaacagcaacaccctcgccctcaagaacaacaccgagatcccccagattcctctcttcatctaccacggcgagctggacgagatcgtccccttcagcggcagccagcgcgcctacaccaactggtgcgagtggggcatcgagtccctcgagttctccaccgccatgctcagcggccacatcaccgagttcttcatgggcgcccctgccgccctcacctgggtcatcgagcgcttcgagggcaagcagcccgtcaagggctgccagaagacgcagcgcctcaccaacctcgactaccccggcacccccgaggccctccagatctacttccaggccgcctacgagagcgtctttcagatggacgtcggccccaacggcgagaacttcaccaagagcgacctccagcgcctcgccaagcgcagcttcaacggcttcacccccatcgacaccagcaacctcaagaagcgctaatag

SEQ ID NO: 56 - protein, 5 aa, Artificial Sequence
  Synthetic peptide motif
GXSXG

SEQ ID NO: 57 - protein, 5 aa, Artificial Sequence
  Synthetic peptide motif
YAXXX

SEQ ID NO: 58 - protein, 5 aa, Artificial Sequence
  Synthetic peptide motif
GYSGG

SEQ ID NO: 59 - protein, 5 aa, Artificial Sequence
  Synthetic peptide motif
GYSQG

SEQ ID NO: 60 - protein, 5 aa, Artificial Sequence
  Synthetic peptide motif
GHSQG

SEQ ID NO: 61 - protein, 5 aa, Artificial Sequence
  Synthetic peptide motif
YAPEL

SEQ ID NO: 62 - protein, 5 aa, Artificial Sequence
  Synthetic peptide motif
YAPDV

SEQ ID NO: 63 - protein, 5 aa, Artificial Sequence
  Synthetic peptide motif
YAPDL

SEQ ID NO: 64 - protein, 5 aa, Artificial Sequence
  Synthetic peptide motif
YAPEI

SEQ ID NO: 65 - protein, 5 aa, Artificial Sequence
  Synthetic peptide motif
YAKEL

SEQ ID NO: 66 - protein, 5 aa, Artificial Sequence
  Synthetic peptide motif
GYSEG

SEQ ID NO: 67 - protein, 5 aa, Artificial Sequence
  Synthetic peptide motif
GXSZG

SEQ ID NO: 68 - protein, 401 aa, Rhodococcus jostii
MKSAVRKIIGSTSAALAVAVGAALLAPPAAQAAPAYPFADPDPFYGAPVDVGARADGDVLASRPVAATGYPTADAWQVKYKSTNSAGAPIAAITTVLVPRGTPANRPLVSYQPFVNALGLECAPSHQLFTGGIQEGPALNALLARGWAVAVPDHLGPTSAYGAAKLGGRLVLDGVRAAQRLPEAGLGGSPVGLAGYSGGAMATNWAAALAPTYAPELNIVGAAQGGTPVNIGELADRLGSTRSQAFGLGFAAAIGLEREYPTRSPLGDGLNGDGLALRAQMSNMCTDSILGAGAGKSLPDLTNGPGILADPSGRSVMDENSVELYPGVPRTPMFVWHGSADPLVAIEPVQNTVARYCSQGVPVQFDVIPGADHGTATMLGAGPAFGYLGDRFAGIPAPSNC